(19) United States
(12) Patent Application Publication
(10) Pub. No.: US 2010/0222396 A1
(43) Pub. Date: Sep. 2, 2010

(54) 4-ARYL-BUTANE-1,3-DIAMIDES

(75) Inventors: Dirk BEHNKE, Basel (CH); Claudia Betschart, Basel (CH); Simona Cotesta, Basel (CH); Marc Gerspacher, Basel (CH); Samuel Hintermann, Basel (CH); Bernard Lucien Roy, Basel (CH); Anette Von Matt, Basel (CH); Jürgen Wagner, Basel (CH)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG, Basel (CH)

(21) Appl. No.: 12/695,462

(22) Filed: Jan. 28, 2010

Related U.S. Application Data
(60) Provisional application No. 61/148,426, filed on Jan. 30, 2009.

(51) Int. Cl. A61K 31/44; C07D 207/30; A61K 31/40; C07D 213/81

(52) U.S. Cl. 514/354; 548/538; 514/423; 546/323

(57) ABSTRACT

The invention relates to compound of the formula I

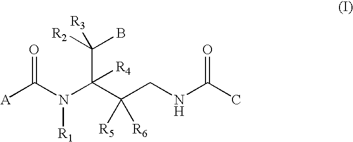

in which the substituents are as defined in the specification; in free form or in salt form; to its preparation, to its use as medicament and to medicaments comprising it.

This application claims priority to U.S. Provisional Application Ser. No. 61/148,426 filed 30 Jan. 2009, the contents of which are incorporated herein by reference in their entirety.

The invention relates to 4-aryl-butane-1,3-diamides, to their preparation, to their use as medicaments and to medicaments comprising them.

Orexins (orexin A/OX-A and orexin B/OX-B), which are also known as hypocretins, are neuropeptides. Orexin A is a 33 amino acid peptide and orexin B is a 28 amino acid peptide (Sakurai T. et al., Cell, 1998, 92, 573-585). Orexins are produced in discrete neurons of the lateral hypothalamus and bind to G-protein-coupled receptors, the orexin receptors (also known as hypocretin receptors): known are the orexin-1 receptor (OX1R) and the orexin-2 receptor (OX2R). The orexin-1 receptor has some selectivity for OX-A, whereas the orexin-2 receptor binds OX-A and OX-B with similar affinity. Orexins regulate states of sleep and wakefulness, opening potentially novel therapeutic approaches for narcolepsy as well as insomnia and other sleep disorders (Chemelli R. M. et al., Cell, 1999, 98, 437-451). Furthermore, orexins were found to stimulate food consumption in rats suggesting a physiological role for these peptides as mediators in the central feedback mechanism that regulates feeding behavior (Sakurai T. et al., Cell, 1998, 92, 573-585). Still furthermore, orexins were shown to play a role in brain reward function/motivation suggesting usefulness to treat substance-related disorders (Harris A. C. et al, Nature, 2005, 437, 556-559). Still furthermore, it has been shown that amyloid beta levels inversely correlate with orexin levels in rodents and humans (brain and/or CSF), and that an orexin receptor antagonist reduces both amyloid beta levels and amyloid plaque load in Alzheimer's transgenic mice, thus suggesting usefulness in the treatment of Alzheimers disease (Kang J. E. et al, Science 2009, 326, 1005-1007).

Orexin receptors may have numerous implications in disorders such as

i) sleep disorders, e.g. sleep apnea, narcolepsy, insomnia, parasomnia, jet lag syndrome, disturbed biological and circadian rhythms; sleep disturbances associated with diseases such as neurological disorders, neuropathic pain and restless leg syndrome;
ii) eating disorders, e.g. appetite and taste disorders;
iii) substance-related disorders, e.g. substance abuse, substance dependence and substance withdrawal disorders, such as nicotine withdrawal or narcotics withdrawal;
iv) Alzheimers disease;
v) psychiatric, neurological and neurodegenerative disorders, e.g. depression; anxiety; addictions, obsessive compulsive disorder; affective neurosis; depressive neurosis; anxiety neurosis; dysthymic disorder; mood disorder; sexual dysfunction; psychosexual dysfunction; sex disorder; schizophrenia; manic depression; delirium; dementia; severe mental retardation and dyskinesias such as Huntington's disease and Tourette syndrome; Parkinson's disease; ischemic or hemorrhagic stroke; migraine; and neurodegenerative disorders including nosological entities such as disinhibition-dementia-parkinsonism-amyotrophy complex; pallido-ponto-nigral degeneration epilepsy; seizure disorders;
vi) cardiovascular diseases, diabetes; asthma; Cushing's syndrome/disease; basophile adenoma; prolactinoma; hyperprolactinemia; hypopituitarism; hypophysis tumor/adenoma; hypothalamic diseases; Froehlich's syndrome; hypophysis diseases, hypothalamic hypogonadism; Kallman's syndrome (anosmia, hyposmia); functional or psychogenic amenorrhea; hypopituitarism; hypothalamic hypothyroidism; hypothalamic-adrenal dysfunction; idiopathic hyperprolactinemia; hypothalamic disorders of growth hormone deficiency; idiopathic growth deficiency; dwarfism; gigantism; acromegaly; heart and lung diseases, acute and congestive heart failure; hypotension; hypertension; urinary retention; osteoporosis; angina pectoris; myocardial infarction; subarachnoid hemorrhage; ulcers; allergies; benign prostatic hypertrophy; chronic renal failure; renal disease; impaired glucose tolerance; vomiting and nausea; inflammatory bowel disease; gastric dyskinesia; gastric ulcers; urinary bladder incontinence e.g. urge incontinence; hyperalgesia; pain; enhanced or exaggerated sensitivity to pain such as hyperalgesia, causalgia, and allodynia; acute pain; burn pain; atypical facial pain; neuropathic pain; back pain; complex regional pain syndrome I and II; arthritic pain; sports injury pain; pain related to infection e.g. HIV, post-chemotherapy pain; post-stroke pain; post-operative pain; neuralgia; conditions associated with visceral pain such as irritable bowel syndrome, migraine and angina; and
vii) other diseases related to general orexin system dysfunction.

Orexin receptor antagonists, are considered to be useful in the treatment of a wide range of disorders, in particular sleep disorders, eating disorders and substance-related disorders.

Therefore, there is a need to provide new orexin receptor antagonists that are good drug candidates. In particular, preferred compounds should bind potently to the orexin receptors (either as OXR1 or OXR2 subtype selective antagonists or as dual OXR1/OXR2 antagonists) whilst showing little affinity for other receptors. They should be well absorbed from the gastrointestinal tract, be sufficiently metabolically stable and possess favorable pharmacokinetic properties. When targeted against receptors in the central nervous system they should cross the blood brain barrier freely and when targeted selectively against receptors in the peripheral nervous system they should not cross the blood brain barrier. They should be non-toxic and demonstrate few side-effects. Furthermore, the ideal drug candidate will be able to exist in a physical form that is stable, non-hygroscopic and easily formulated.

The compounds of the invention are orexin receptor antagonists and are therefore potentially useful in the treatment of a wide range of disorders, particularly sleep disorders, eating disorders, substance-related disorders and Alzheimers disease.

In a first aspect, the invention relates to a compound of the formula I

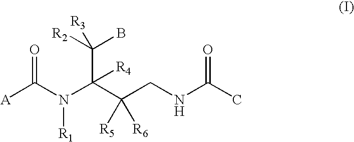

wherein
R₁ is C₁₋₆alkyl, C₁₋₆halogenalkyl, C₃₋₆cycloalkyl or C₃₋₆cycloalkyl(C₁₋₄alkyl);
R₂, R₃, R₅ and R₆ are each independently selected from hydrogen, halogen, hydroxyl, C₁₋₆alkyl, C₁₋₆halogenalkyl, C₃₋₆cycloalkyl, C₃₋₆cycloalkyl(C₁₋₄alkyl), C₁₋₆alkoxy, or C₁₋₆halogenalkoxy;
or R₂ and R₃ together are oxo;
or R₂ and R₃ taken together with the carbon atom to which they are bound form a C₃₋₆cycloalkyl;
or R₅ and R₆ together are oxo;
or R₅ and R₆ taken together with the carbon atom to which they are bound form a C₃₋₆cycloalkyl;
R₄ is hydrogen, C₁₋₆alkyl or hydroxyl;
A is phenyl, which may be substituted once or twice by R₇;
each R₇ independently is halogen, C₁₋₆alkyl, C₁₋₆halogenalkyl, C₃₋₆cycloalkyl, C₃₋₆cycloalkyl(C₁₋₄alkyl), C₁₋₆alkoxy, or C₁₋₆halogenalkoxy;
or two R₇ at adjacent ring atoms form a C₃₋₄alkylene group, wherein 1-2 carbon atoms may be replaced by X₁, and wherein the C₃₋₄alkylene group may be substituted once or more than once by R₈;
each X₁ independently is —O— or —N(R₉)—;
each R₉ independently is hydrogen or C₁₋₆alkyl;
each R₈ independently is halogen or C₁₋₆alkyl;
or two R₇ at adjacent ring atoms are —CH═CH—CH═CH—;
or two R₇ at adjacent ring atoms are —CH═CH—X₂—;

### X₂ is —O— or —N(R₁₀)—;

R₁₀ is hydrogen or C₁₋₆alkyl;

### B is

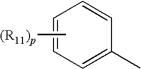

p is 0 or 1;
R₁₁ is halogen, C₁₋₆alkyl, C₁₋₆halogenalkyl, C₃₋₆cycloalkyl, C₁₋₆alkoxy, or C₁₋₆halogenalkoxy; and
C is a five- to ten-membered monocyclic or fused polycyclic aromatic ring system which may contain from 1 to 4 hetero atoms selected from nitrogen, oxygen and sulfur, wherein the ring system may contain not more than 2 oxygen atoms and not more than 2 sulfur atoms, and wherein the ring system may be substituted once or more than once by R₁₂, and wherein a substituent on a nitrogen in a heterocyclic ring system may not be halogen;
each R₁₂ independently is C₁₋₆alkyl, C₁₋₆halogenalkyl, C₁₋₆alkoxy, C₁₋₆halogenalkoxy, halogen, cyano or a three- to six-membered monocyclic ring system which may be aromatic, saturated or partially saturated and which may contain from 1 to 4 hetero atoms selected from nitrogen, oxygen and sulfur, and wherein each ring system may contain not more than 2 oxygen atoms and not more than 2 sulfur atoms, and wherein each ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen;
or two R₁₂ at adjacent ring atoms form a C₃₋₄alkylene group, wherein 1-2 carbon atoms may be replaced by X₃, and wherein the C₃₋₄alkylene group may be substituted once or more than once by R₁₃;
each X₃ independently is —O— or —N(R₁₄)—;
each R₁₄ independently is hydrogen or C₁₋₆alkyl;
each R₁₃ independently is halogen or C₁₋₆alkyl;
or C is C₁₋₆alkyl, C₁₋₆halogenalkyl, C₃₋₆cycloalkyl or C₃₋₆cycloalkyl(C₁₋₄alkyl);
in free form or in salt form.

Unless indicated otherwise, the expressions used in this invention have the following meaning:

“Alkyl” represents a straight-chain or branched-chain alkyl group, for example, methyl, ethyl, n- or iso-propyl, n-, iso-, sec- or tert-butyl, n-pentyl, n-hexyl; C₁₋₆alkyl preferably represents a straight-chain or branched-chain C₁₋₄alkyl with particular preference given to methyl, ethyl, n-propyl, iso-propyl and tert-butyl.

Each alkyl part of “alkoxy”, “alkoxyalkyl”, “alkoxycarbonylalkyl” and “halogenalkyl” and so on shall have the same meaning as described in the above-mentioned definition of “alkyl”, especially regarding linearity and preferential size.

“C₃₋₆cycloalkyl” represents a saturated alicyclic moiety having from three to six carbon atoms. This term refers to groups such as cyclopropyl, cyclobutyl, cyclopentyl and cyclohexyl.

A substituent being substituted “once or more than once”, for example as defined for C, is preferably substituted by one to three substituents.

Halogen is generally fluorine, chlorine, bromine or iodine; preferably fluorine, chlorine or bromine. Halogenalkyl groups preferably have a chain length of 1 to 4 carbon atoms and are, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,2-trichloroethyl, 1,1,2,2-tetrafluoroethyl, 2,2,3,3-tetrafluoropropyl, 2,2,3,3,3-pentafluoropropyl or 2,2,3,4,4,4-hexafluorobutyl; preferably —CF₃, —CHF₂, —CH₂F, —CHF—CH₃, —CF₂CH₃, or —CH₂CF₃.

In the context of the invention, the definitions of “two R₇ at adjacent ring atoms form a C₃₋₄alkylene group, wherein 1-2 carbon atoms may be replaced by X₁” or “two R₁₂ at adjacent ring atoms form a C₃₋₄alkylene group, wherein 1-2 carbon atoms may be replaced by X₃” encompass —CH₂—CH₂—CH₂—, —CH₂—CH₂—CH₂—CH₂—, —O—CH₂—O—, —O—CH₂—CH₂—O— and —CH₂—CH₂—NH—. An example of a substituted group is —CH₂—CH₂—N(CH₃)—.

In the context of the invention, the definition of C as a “five- to ten-membered monocyclic or fused polycyclic aromatic ring system” encompasses a C₆- or C₁₀-aromatic hydrocarbon group or a five- to ten-membered heterocyclic aromatic ring system. “Polycyclic” means preferably bicyclic.

In the context of the invention, the definition of R₁₂ as a “three- to six-membered monocyclic ring system” encompasses a C₆₋aromatic hydrocarbon group, a five- to six-membered heterocyclic aromatic ring system and a three- to six-membered monocyclic aliphatic or heterocyclic ring system.

A C₆- or C₁₀-aromatic hydrocarbon group is typically phenyl or naphthyl, especially phenyl.

Preferably, but also depending on substituent definition, “five- to ten-membered heterocyclic aromatic ring systems” consist of 5 to 10 ring atoms of which 1-3 ring atoms are hetero atoms. Such heterocyclic aromatic ring systems may be present as a single ring system or as bicyclic or tricyclic ring systems; preferably as single ring systems or as benz-annelated ring systems. Bicyclic or tricyclic ring systems may be formed by annelation of two or more rings, or by a bridging atom, e.g. oxygen, sulfur, nitrogen.

Examples of heterocyclic ring systems are: imidazo[2,1-b]thiazole, pyrrole, pyrroline, pyrrolidine, pyrazole, pyrazoline, pyrazolidine, imidazole, imidazoline, imidazolidine, triazole, triazoline, triazolidine, tetrazole, furane, dihydrofurane, tetrahydrofurane, furazane (oxadiazole), dioxolane, thiophene, dihydrothiophene, tetrahydrothiophene, oxazole, oxazoline, oxazolidine, isoxazole, isoxazoline, isoxazolidine, thiazole, thiazoline, thiazolidine, isothiazole, isothiazoline, isothiazolidine, thiadiazole, thiadiazoline, thiadiazolidine, pyridine, piperidine, pyridazine, pyrazine, piperazine, triazine, pyrane, tetrahydropyrane, thiopyrane, tetrahydrothiopyrane, oxazine, thiazine, dioxine, morpholine, purine, pteridine, and the corresponding benz-annelated heterocycles, e.g. indole, isoindole, coumarin, isoquinoline, quinoline and the like. Preferred heterocycles are: imidazo[2,1-b]thiazole, oxazole, isoxazole, thiazole, isothiazole, triazole, pyrrole, furane, tetrahydrofurane, pyridine, pyrimidine, imidazole or pyrazole.

The compounds of formula I exist in optically active form or in form of mixtures of optical isomers, e.g. in form of racemic mixtures or diastereomeric mixtures. In particular, further asymmetrical carbon atom(s) may be present in the compounds of formula I and their salts. All optical isomers and their mixtures, including the racemic mixtures, are embraced by the invention.

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the invention and includes geometric isomers. It is understood that a substituent may be attached at a chiral center of a carbon atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. The compounds described herein contain one or more asymmetric centers and may thus give rise to enantiomers, diastereomers, and other stereoisomeric forms that may be defined, in terms of absolute stereochemistry, as (R)- or (S)-. The invention is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration.

Any asymmetric atom (e.g. carbon or the like) of the compound(s) of the invention can be present in racemic or enantiomerically enriched, for example the (R)-, (S)- or (R, S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis-(Z)- or trans-(E)-form.

Accordingly, as used herein a compound of the invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Depending on substituent definition, compounds of formula I may occur in various tautomeric forms. All tautomeric forms of the compounds of formula I are embraced by the invention.

Compounds of formula I may exist in free form or as a salt. In this specification, unless otherwise indicated, language such as “compound of formula I” is to be understood as embracing the compounds in any form, for example free or acid addition salt form. Salts which are unsuitable for pharmaceutical uses but which can be employed, for example, for the isolation or purification of free compounds of formula I, such as picrates or perchlorates, are also included. For therapeutic use, only pharmaceutically acceptable salts or free compounds are employed (where applicable in the form of pharmaceutical preparations), and are therefore preferred. Salts are preferably physiologically acceptable salts, formed by the addition of an acid.

As used herein, the term “pharmaceutically acceptable salts” refers to salts that retain the biological effectiveness and properties of the compounds of this invention and, which typically are not biologically or otherwise undesirable. The compounds of the invention may be capable of forming acid salts by virtue of the presence of suitable groups, such as amino groups.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

The pharmaceutically acceptable salts of the invention can be synthesized from a parent compound by conventional chemical methods. Generally, such salts can be prepared by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

The invention includes all pharmaceutically acceptable isotopically-labeled compounds of the invention, i.e. compounds of formula (I), wherein (1) one or more atoms are replaced by atoms having the same atomic number, but an atomic mass or mass number different from the atomic mass or mass number usually found in nature, and/or (2) the isotopic ratio of one or more atoms is different from the naturally occurring ratio.

Examples of isotopes suitable for inclusion in the compounds of the invention comprises isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labeled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Tomography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula I with the co-crystal former under crystallization conditions and isolating co-crystals thereby formed. Suitable co-crystal formers include those described in WO 2004/078163. Hence the invention further provides co-crystals comprising a compound of formula (I).

Compounds of the invention are either obtained in the free form, as a salt thereof, or as prodrug derivatives thereof.

The invention also provides pro-drugs of the compounds of the invention that converts in vivo to the compounds of the invention. A pro-drug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds, which are inactive or have low activity compared to the corresponding active drug compound, that contain one or more protective groups and are converted to an active form by metabolism or solvolysis. Both the active drug form and any released metabolic products should have acceptably low toxicity.

Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of hydroxyl groups with lipophilic carboxylic acids (e.g., a carboxylic acid having at least one lipophilic moiety).

Exemplary prodrugs are, e.g., O-acyl derivatives of alcohols. Preferred are pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

Furthermore, the compounds of the invention, including their salts, can also be obtained in the form of their hydrates, or include other solvents used for their crystallization.

Preferred substituents, preferred ranges of numerical values or preferred ranges of the radicals present in compounds of the formula I, IA, IB and the corresponding intermediate compounds are defined below. The definition of the substituents applies to the end-products as well as to the corresponding intermediates. The definitions of the substituents may be combined at will, e.g. preferred substituents R¹ and particularly preferred substituents R².

In especially preferred embodiments, the invention relates to one or more than one of the compounds of the formula I mentioned in the Examples hereinafter, in free form or in salt form.

One class of compounds of the invention, are compounds of formula IA

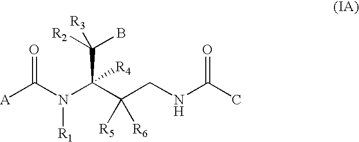

wherein R₁, R₂, R₃, R₄, R₅, R₆, A, B and C are as defined under formula (I).

One class of compounds of the invention, are compounds of formula IB

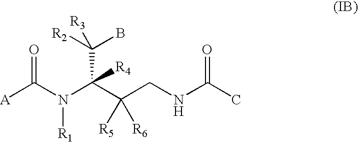

wherein R₁, R₂, R₃, R₄, R₅, R₆, A, B and C are as defined under formula (I).

In one class of compounds of the invention, R₁ is C₁₋₆alkyl, for example, methyl, ethyl or n-propyl. In one class of compounds of the invention, R₁ is methyl.

In one class of compounds of the invention, R₂, R₃, R₅ and R₆ are each independently selected from hydrogen, halogen, and C₁₋₆alkyl. In one class of compounds of the invention, R₂, R₃, R₅ and R₆ are each hydrogen.

In one class of compounds of the invention, R₄ is hydrogen or C₁₋₆alkyl. In one class of compounds of the invention, R₄ is hydrogen.

In one class of compounds of the invention, A is a ring system selected from

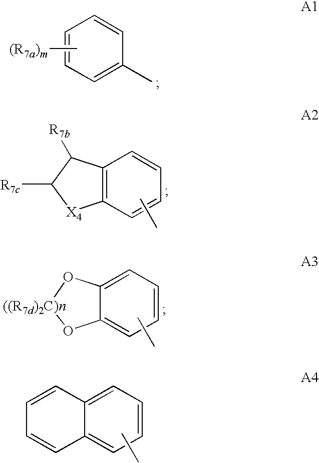

m is 0, 1 or 2;
each R₇ₐ independently is halogen, C₁₋₆alkyl, C₁₋₆halogenalkyl, C₃₋₆cycloalkyl, C₃₋₆cycloalkyl(C₁₋₄alkyl), C₁₋₆alkoxy, or C₁₋₆halogenalkoxy;
R_{7b} and R_{7c} are each independently hydrogen or C₁₋₆alkyl, or are together a bond;
X₄ is oxygen or —N(R₇ₑ)—;
R₇ₑ is hydrogen or C₁₋₆alkyl;
n is 1 or 2;
each R_{7d} is independently hydrogen, halogen or C₁₋₆alkyl.

In one class of compounds of the invention, A is A1.

In one subclass of said class, m is 0.

In one subclass of said class, m is 1. In one subclass of said class, m is 2.

In one class of compounds of the invention, R₇ₐ is halogen, C₁₋₆alkyl, C₁₋₆halogenalkyl or C₁₋₆alkoxy. In one subclass, R₇ₐ is halogen, for example fluoro. In one subclass, R₇ₐ is C₁₋₆halogenalkyl, for example, trifluoromethyl. In one subclass, R₇ₐ is C₁₋₆alkoxy, for example, methoxy.

In one class of compounds of the invention, A is A2. A2 is selected from the groups A2a, A2b, A2c and A2d

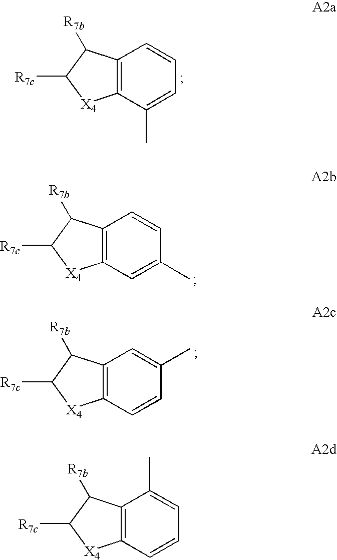

In one class of compounds of the invention, A is A2a.

In one class of compounds of the invention, A is A2b.

In one class of compounds of the invention, A is A2c.

In one class of compounds of the invention, A is A2d.

In one subclass of said class, wherein A is A2, R_{7b} and R_{7c} are each hydrogen or C₁₋₆alkyl, X₄ is oxygen or —N(R₇ₑ)—, and R₇ₑ is hydrogen or C₁₋₆alkyl. R₇ₑ, for example, is hydrogen or methyl.

In another subclass of said class, wherein A is A2, R_{7b} and R_{7c} are together a bond, X₄ is oxygen or —N(R₇ₑ)—, and R₇ₑ is hydrogen or C₁₋₆alkyl. R₇ₑ, for example, is hydrogen or methyl.

In one class of compounds of the invention, A is A3. In one class of compounds of the invention, n is 1. In one class of compounds of the invention, n is 2. In one class of compounds of the invention, each R_{7d} is hydrogen. In one class of compounds of the invention, A is benzo[1,3]dioxol-5-yl. In one class of compounds of the invention, A is benzo[1,3]dioxol-4-yl. In one class of compounds of the invention, A is 2,3-dihydro-benzo[1,4]dioxin-5-yl. In one class of compounds of the invention, A is 2,3-dihydro-benzo[1,4]dioxin-6-yl.

In one class of compounds of the invention, A is A4. In one class of compounds of the invention, A is 1-naphthyl. In one class of compounds of the invention, A is 2-naphthyl.

In one class of compounds of the invention, p is 0.

In one class of compounds of the invention, p is 1.

In one class of compounds of the invention, p is 1 and R₁₁ is halogen or C₁₋₆alkyl.

In one class of compounds of the invention, C is a five- to ten-membered monocyclic or fused polycyclic aromatic ring system which may contain from 1 to 4 hetero atoms selected from nitrogen, oxygen and sulfur, wherein the ring system may contain not more than 2 oxygen atoms and not more than 2 sulfur atoms, and wherein the ring system may be substituted once or more than once by R₁₂, and wherein a substituent on a nitrogen in a heterocyclic ring system may not be halogen;

each R₁₂ independently is C₁₋₆alkyl, C₁₋₆halogenalkyl, C₁₋₆alkoxy, C₁₋₆halogenalkoxy, halogen, cyano or a three- to six-membered monocyclic ring system which may be aromatic, saturated or partially saturated and which may contain from 1 to 4 hetero atoms selected from nitrogen, oxygen and sulfur, and wherein each ring system may contain not more than 2 oxygen atoms and not more than 2 sulfur atoms, and wherein each ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen;
or two R₁₂ at adjacent ring atoms form a C₃₋₄alkylene group, wherein 1-2 carbon atoms may be replaced by X₃, and wherein the C₃₋₄alkylene group may be substituted once or more than once by R₁₃;
each X₃ independently is —O— or —N(R₁₄)—;
each R₁₄ independently is hydrogen or C₁₋₆alkyl;
each R₁₃ independently is halogen or C₁₋₆alkyl.

In one class of compounds of the invention, C is phenyl, which may be substituted once or more than once by R₁₂.

In one class of compounds of the invention, C is a five- to six-membered monocyclic aromatic ring system which contains from 1 to 4 hetero atoms selected from nitrogen, oxygen and sulfur, and wherein the ring system may be substituted once or more than once by R₁₂. In a subclass of said class, C is pyridyl, for example 2-, 3- and 4-pyridyl, or thiazolyl, for example, 2-, 4- and 5-thiazolyl, both of which may be substituted once or more than once by R₁₂. In a subclass of said class, C is 2-pyridyl which may be substituted once or more than once by R₁₂. In a subclass of said class, C is 4-thiazolyl which may be substituted once or more than once by R₁₂.

In one class of compounds of the invention, C is an eight- to ten-membered bicyclic aromatic ring system which contains from 1 to 4 hetero atoms selected from nitrogen, oxygen and sulfur, and wherein the ring system may be substituted once or more than once by R₁₂. In a subclass of said class, C is C1

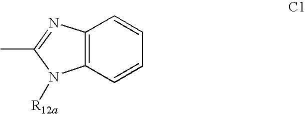

wherein R₁₂ₐ is C₁₋₆alkyl, for example methyl.

In one class of compounds of the invention, each R₁₂ independently is C₁₋₆alkyl or halogen. In one class of compounds of the invention, each R₁₂ independently is C₁₋₆alkyl or a three- to six-membered monocyclic ring system which may be aromatic, saturated or partially saturated and which may contain from 1 to 4 hetero atoms selected from nitrogen, oxygen and sulfur, and wherein each ring system may contain not more than 2 oxygen atoms and not more than 2 sulfur atoms, and wherein each ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen.

In one class of compounds of the invention, each R₁₂ independently is C₁₋₆alkyl or phenyl, which may be substituted once or more than once by halogen.

In one class of compounds of the invention, two R₁₂ at adjacent ring atoms form a C₃₋₄alkylene group, wherein 1-2 carbon atoms may be replaced by X₃, and wherein the C₃₋₄alkylene group may be substituted once or more than once by R₁₃.

In one class of compounds of the invention, C is C₁₋₆alkyl, C₁₋₆halogenalkyl, C₃₋₆cycloalkyl or C₃₋₆cycloalkyl(C₁₋₄alkyl). In a subclass, C is C₁₋₆alkyl.

One class of compounds of the invention, are compounds of formula IA

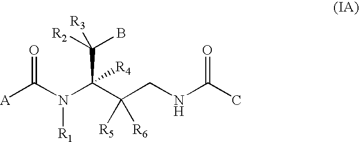

wherein R₁ is C₁₋₆alkyl and R₂, R₃, R₄, R₅ and R₆ are each hydrogen;
A is a ring system selected from

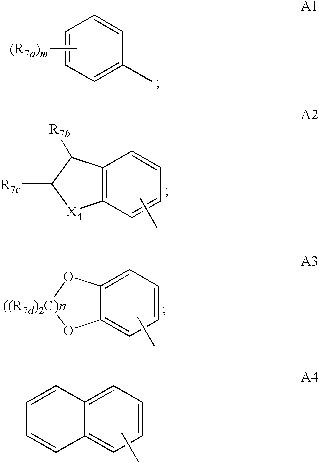

m is 0, 1 or 2;
each R₇ₐ independently is halogen, C₁₋₆alkyl, C₁₋₆halogenalkyl, C₃₋₆cycloalkyl, C₃₋₆cycloalkyl(C₁₋₄alkyl), C₁₋₆alkoxy, or C₁₋₆halogenalkoxy;
R_{7b} and R_{7c} are each independently hydrogen or C₁₋₆alkyl, or are together a bond;
X₄ is oxygen or —N(R₇ₑ)—;
R₇ₑ is hydrogen or C₁₋₆alkyl;
n is 1 or 2;
each R_{7d} is independently hydrogen, halogen or C₁₋₆alkyl;

### B is

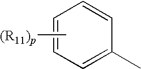

p is 0;
C is a five- to ten-membered monocyclic or fused polycyclic aromatic ring system which may contain from 1 to 4 hetero atoms selected from nitrogen, oxygen and sulfur, wherein the ring system may contain not more than 2 oxygen atoms and not more than 2 sulfur atoms, and wherein the ring system may be substituted once or more than once by R₁₂, and wherein a substituent on a nitrogen in a heterocyclic ring system may not be halogen;
each R₁₂ independently is C₁₋₆alkyl, C₁₋₆halogenalkyl, C₁₋₆alkoxy, C₁₋₆halogenalkoxy, halogen, cyano or a three- to six-membered monocyclic ring system which may be aromatic, saturated or partially saturated and which may contain from 1 to 4 hetero atoms selected from nitrogen, oxygen and sulfur, and wherein each ring system may contain not more than 2 oxygen atoms and not more than 2 sulfur atoms, and wherein each ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen;
or two R₁₂ at adjacent ring atoms form a C₃₋₄alkylene group, wherein 1-2 carbon atoms may be replaced by X₃, and wherein the C₃₋₄alkylene group may be substituted once or more than once by R₁₃;
each X₃ independently is —O— or —N(R₁₄)—;
each R₁₄ independently is hydrogen or C₁₋₆alkyl; and
each R₁₃ independently is halogen or C₁₋₆alkyl.

One class of compounds of the invention, are compounds of formula IA

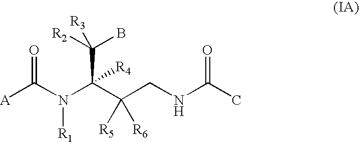

wherein R₁ is C₁₋₆alkyl and R₂, R₃, R₄, R₅ and R₆ are each hydrogen;

### A is

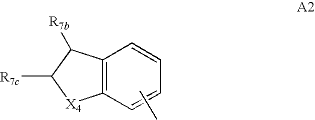

R_{7b} and R_{7c} are each independently hydrogen or C₁₋₆alkyl, or are together a bond;
X₄ is oxygen or —N(R₇ₑ)—;
R₇ₑ is hydrogen or C₁₋₆alkyl;

### B is

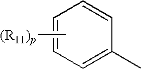

p is 0;
C is a five- to six-membered monocyclic aromatic ring system which may contain from 1 to 4 hetero atoms selected from nitrogen, oxygen and sulfur, wherein the ring system may contain not more than 2 oxygen atoms and not more than 2 sulfur atoms, and wherein the ring system may be substituted once or more than once by R₁₂, and wherein a substituent on a nitrogen in a heterocyclic ring system may not be halogen; and
each R₁₂ independently is C₁₋₆alkyl, C₁₋₆halogenalkyl, C₁₋₆alkoxy, C₁₋₆halogenalkoxy, halogen or cyano. In one embodiment of said class, C is pyrrole.

In one embodiment, the invention provides a compound selected from
Pyridine-2-carboxylic acid {3-[(benzo[1,3]dioxole-5-carbonyl)-methyl-amino]-4-phenyl-butyl}-amide;
Quinoline-4-carboxylic acid [3-(benzoyl-methyl-amino)-4-phenyl-butyl]-amide;
1-Methyl-1H-benzoimidazole-2-carboxylic acid {3-[(1H-indole-4-carbonyl)-methyl-amino]-4-phenyl-butyl}-amide;
Pyridine-2-carboxylic acid {3-[methyl-(3-trifluoromethyl-benzoyl)-amino]-4-phenyl-butyl}-amide;
Pyridine-2-carboxylic acid {3-[(3,5-bis-trifluoromethyl-benzoyl)-methyl-amino]-4-phenyl-butyl}-amide;
Pyridine-2-carboxylic acid {3-[methyl-(naphthalene-1-carbonyl)-amino]-4-phenyl-butyl}-amide;
1H-Indole-5-carboxylic acid {1-benzyl-3-[(pyridine-2-carbonyl)-amino]-propyl}-methyl-amide;
Pyridine-2-carboxylic acid {3-[(benzo[1,3]dioxole-5-carbonyl)-ethyl-amino]-4-phenyl-butyl}-amide;
Pyridine-2-carboxylic acid {3-[(3,4-dimethoxy-benzoyl)-methyl-amino]-4-phenyl-butyl}-amide;
Pyridine-2-carboxylic acid {3-[(3,5-bis-trifluoromethyl-benzoyl)-ethyl-amino]-4-phenyl-butyl}-amide;
1H-Indole-4-carboxylic acid {1-benzyl-3-[(pyridine-2-carbonyl)-amino]-propyl}-methyl-amide;
1-Methyl-1H-benzoimidazole-2-carboxylic acid {3-[(3,5-bis-trifluoromethyl-benzoyl)-methyl-amino]-4-phenyl-butyl}-amide;
Pyridine-2-carboxylic acid {3-[(benzo[1,3]dioxole-5-carbonyl)-propyl-amino]-4-phenyl-butyl}-amide;
1H-Indole-2-carboxylic acid {3-[(benzo[1,3]dioxole-5-carbonyl)-methyl-amino]-4-phenyl-butyl}-amide;
N-[3-(Benzoyl-methyl-amino)-4-phenyl-butyl]-benzamide;
Pyridine-2-carboxylic acid [3-[(3,5-bis-trifluoromethyl-benzoyl)-methyl-amino]-4-(4-chloro-phenyl)-butyl]-amide;
N-(3-Benzoylamino-1-benzyl-propyl)-N-methyl-3,5-bis-trifluoromethyl-benzamide;
1-Methyl-1H-benzoimidazole-2-carboxylic acid {3-[methyl-(naphthalene-1-carbonyl)-amino]-4-phenyl-butyl}-amide;
1-Methyl-1H-benzoimidazole-2-carboxylic acid {3-[(benzo[1,3]dioxole-4-carbonyl)-methyl-amino]-4-phenyl-butyl}-amide;
Pyridine-2-carboxylic acid {3-[(benzofuran-5-carbonyl)-methyl-amino]-4-phenyl-butyl}-amide;
Pyridine-2-carboxylic acid {3-[methyl-(naphthalene-2-carbonyl)-amino]-4-phenyl-butyl}-amide;
Pyridine-2-carboxylic acid {3-[(3,5-bis-trifluoromethyl-benzoyl)-propyl-amino]-4-phenyl-butyl}-amide;
2,3-Dihydro-1H-indole-5-carboxylic acid {1-benzyl-3-[(pyridine-2-carbonyl)-amino]-propyl}-methyl-amide;
1-Methyl-1H-benzoimidazole-2-carboxylic acid {4-(4-fluoro-phenyl)-3-[methyl-(naphthalene-1-carbonyl)-amino]-butyl}-amide;
Pyridine-2-carboxylic acid [3-[(benzo[1,3]dioxole-5-carbonyl)-methyl-amino]-4-(4-chloro-phenyl)-butyl]-amide;
1-Methyl-1H-benzoimidazole-2-carboxylic acid {4-(4-chloro-phenyl)-3-[methyl-(naphthalene-1-carbonyl)-amino]-butyl}-amide;
5-(2-Fluoro-phenyl)-2-methyl-thiazole-4-carboxylic acid [3-(benzoyl-methyl-amino)-4-phenyl-butyl]-amide;
Pyridine-2-carboxylic acid {4-(4-fluoro-phenyl)-3-[methyl-(naphthalene-1-carbonyl)-amino]-butyl}-amide;
Pyridine-2-carboxylic acid {3-[(2,3-dihydro-benzofuran-5-carbonyl)-methyl-amino]-4-phenyl-butyl}-amide;
Pyridine-2-carboxylic acid {3-[(benzo[1,3]dioxole-4-carbonyl)-methyl-amino]-4-phenyl-butyl}-amide;
1-Methyl-1H-benzoimidazole-2-carboxylic acid {3-[(benzo[1,3]dioxole-5-carbonyl)-methyl-amino]-4-phenyl-butyl}-amide;
Benzo[1,3]dioxole-4-carboxylic acid [3-(benzoyl-methyl-amino)-4-phenyl-butyl]-amide;
6-Methyl-imidazo[2,1-b]thiazole-5-carboxylic acid [3-(benzoyl-methyl-amino)-4-phenyl-butyl]-amide;
Pyridine-2-carboxylic acid {3-[(3,5-difluoro-benzoyl)-methyl-amino]-4-phenyl-butyl}-amide;
Pyridine-2-carboxylic acid {4-(4-chloro-phenyl)-3-[methyl-(naphthalene-1-carbonyl)-amino]-butyl}-amide;
1-Methyl-1H-indole-7-carboxylic acid {1-benzyl-3-[(pyridine-2-carbonyl)-amino]-propyl}-methyl-amide;
Pyridine-2-carboxylic acid [3-(benzoyl-methyl-amino)-4-phenyl-butyl]-amide;
1H-Indole-7-carboxylic acid {1-benzyl-3-[(pyridine-2-carbonyl)-amino]-propyl}-methyl-amide;
N-(1-Benzyl-3-pentanoylamino-propyl)-N-methyl-3,5-bis-trifluoromethyl-benzamide;
1-Methyl-1H-indole-4-carboxylic acid {1-benzyl-3-[(pyridine-2-carbonyl)-amino]-propyl}-methyl-amide;
Pyridine-2-carboxylic acid {3-[(3,5-dimethoxy-benzoyl)-methyl-amino]-4-phenyl-butyl}-amide;
Pyridine-2-carboxylic acid {3-[(4-methoxy-benzoyl)-methyl-amino]-4-phenyl-butyl}-amide;
1-Methyl-1H-benzoimidazole-2-carboxylic acid {3-[methyl-(naphthalene-2-carbonyl)-amino]-4-phenyl-butyl}-amide;
Pyridine-2-carboxylic acid {3-[(benzo[1,3]dioxole-4-carbonyl)-ethyl-amino]-4-phenyl-butyl}-amide;
Pyridine-2-carboxylic acid {3-[(2,3-dihydro-benzofuran-7-carbonyl)-methyl-amino]-4-phenyl-butyl}-amide;
1-Methyl-1H-benzoimidazole-2-carboxylic acid [3-(benzoyl-methyl-amino)-4-phenyl-butyl]-amide;
Quinoline-8-carboxylic acid [3-(benzoyl-methyl-amino)-4-phenyl-butyl]-amide;
1-Methyl-1H-benzoimidazole-2-carboxylic acid {4-(4-chloro-phenyl)-3-[methyl-(naphthalene-2-carbonyl)-amino]-butyl}-amide;
Pyridine-2-carboxylic acid [3-[(benzo[1,3]dioxole-5-carbonyl)-methyl-amino]-4-(4-fluoro-phenyl)-butyl]-amide;
Pyridine-2-carboxylic acid {3-[(3-methoxy-benzoyl)-methyl-amino]-4-phenyl-butyl}-amide;
Pyridine-2-carboxylic acid {4-(4-chloro-phenyl)-3-[methyl-(naphthalene-2-carbonyl)-amino]-butyl}-amide;
Pyridine-2-carboxylic acid {3-[(2,3-dihydro-benzo[1,4]dioxine-5-carbonyl)-methyl-amino]-4-phenyl-butyl}-amide;
1-Methyl-2,3-dihydro-1H-indole-5-carboxylic acid {1-benzyl-3-[(pyridine-2-carbonyl)-amino]-propyl}-methyl-amide;
1-Methyl-1H-benzoimidazole-2-carboxylic acid [3-[(benzo[1,3]dioxole-4-carbonyl)-methyl-amino]-4-(4-fluoro-phenyl)-butyl]-amide;
1-Methyl-1H-benzoimidazole-2-carboxylic acid {4-(4-fluoro-phenyl)-3-[methyl-(naphthalene-2-carbonyl)-amino]-butyl}-amide;
Pyridine-2-carboxylic acid {4-(4-fluoro-phenyl)-3-[methyl-(naphthalene-2-carbonyl)-amino]-butyl}-amide;
Pyridine-2-carboxylic acid {3-[(benzo[1,3]dioxole-4-carbonyl)-propyl-amino]-4-phenyl-butyl}-amide;
1-Methyl-1H-indole-5-carboxylic acid {3-[(3,5-bis-trifluoromethyl-benzoyl)-methyl-amino]-4-phenyl-butyl}-amide;
Benzo[1,3]dioxole-5-carboxylic acid {3-[(benzo[1,3]dioxole-5-carbonyl)-amino]-1-benzyl-propyl}-methyl-amide;
Pyridine-2-carboxylic acid [3-[(benzo[1,3]dioxole-4-carbonyl)-methyl-amino]-4-(4-fluoro-phenyl)-butyl]-amide;
1H-Indole-6-carboxylic acid {1-benzyl-3-[(pyridine-2-carbonyl)-amino]-propyl}-methyl-amide;
Pyridine-2-carboxylic acid [3-[(benzo[1,3]dioxole-4-carbonyl)-methyl-amino]-4-(4-chloro-phenyl)-butyl]-amide;
Pyridine-2-carboxylic acid [3-(benzoyl-methyl-amino)-4-(4-fluoro-phenyl)-butyl]-amide;
2,3-Dihydro-benzofuran-7-carboxylic acid [3-(benzoyl-methyl-amino)-4-phenyl-butyl]-amide;
Pyridine-2-carboxylic acid [3-(benzoyl-methyl-amino)-2-hydroxy-4-phenyl-butyl]-amide;
Pyridine-2-carboxylic acid [3-(benzoyl-methyl-amino)-4-(4-chloro-phenyl)-butyl]-amide;
Pyridine-2-carboxylic acid {3-[(2,3-dihydro-benzo[1,4]dioxine-6-carbonyl)-methyl-amino]-4-phenyl-butyl}-amide;
Benzo[1,3]dioxole-5-carboxylic acid {3-[(benzo[1,3]dioxole-5-carbonyl)-methyl-amino]-2-hydroxy-4-phenyl-butyl}-amide;
Pyridine-2-carboxylic acid {3-[methyl-(4-trifluoromethyl-benzoyl)-amino]-4-phenyl-butyl}-amide;
1-Methyl-1H-indole-2-carboxylic acid {3-[(benzo[1,3]dioxole-5-carbonyl)-methyl-amino]-4-phenyl-butyl}-amide;
1-Methyl-1H-indole-2-carboxylic acid {3-[(benzo[1,3]dioxole-5-carbonyl)-methyl-amino]-4-phenyl-butyl}-amide;
Pyridine-2-carboxylic acid {3-[(3,4-bis-trifluoromethyl-benzoyl)-methyl-amino]-4-phenyl-butyl}-amide;
1-Methyl-1H-imidazole-2-carboxylic acid {3-[(benzo[1,3]dioxole-5-carbonyl)-methyl-amino]-4-phenyl-butyl}-amide;
1-Methyl-1H-benzoimidazole-2-carboxylic acid {3-[(3-methoxy-benzoyl)-methyl-amino]-4-phenyl-butyl}-amide;
1-Methyl-1H-benzoimidazole-2-carboxylic acid {3-[(3,4-dimethoxy-benzoyl)-methyl-amino]-4-phenyl-butyl}-amide;
1-Methyl-1H-benzoimidazole-2-carboxylic acid {3-[(1H-indole-5-carbonyl)-methyl-amino]-4-phenyl-butyl}-amide;
6-Methyl-imidazo[2,1-b]thiazole-5-carboxylic acid {3-[(benzo[1,3]dioxole-5-carbonyl)-methyl-amino]-4-phenyl-butyl}-amide;
5-(2-Fluoro-phenyl)-2-methyl-thiazole-4-carboxylic acid {3-[(benzo[1,3]dioxole-5-carbonyl)-methyl-amino]-4-phenyl-butyl}-amide;
1-Methyl-1H-indole-5-carboxylic acid {1-benzyl-3-[(pyridine-2-carbonyl)-amino]-propyl}-methyl-amide;
1-Methyl-1H-indole-6-carboxylic acid {1-benzyl-3-[(pyridine-2-carbonyl)-amino]-propyl}-methyl-amide;
N-[3-(Benzoyl-methyl-amino)-4-phenyl-butyl]-nicotinamide;
Quinoline-2-carboxylic acid [3-(benzoyl-methyl-amino)-4-phenyl-butyl]-amide;
Quinoline-7-carboxylic acid [3-(benzoyl-methyl-amino)-4-phenyl-butyl]-amide;
Isoquinoline-3-carboxylic acid [3-(benzoyl-methyl-amino)-4-phenyl-butyl]-amide;
1-Methyl-1H-benzoimidazole-2-carboxylic acid {3-[(4-methoxy-benzoyl)-methyl-amino]-4-phenyl-butyl}-amide;
1-Methyl-1H-pyrrole-2-carboxylic acid [3-(benzoyl-methyl-amino)-4-phenyl-butyl]-amide;
1-Methyl-1H-imidazole-2-carboxylic acid [3-(benzoyl-methyl-amino)-4-phenyl-butyl]-amide;
Benzo[1,3]dioxole-5-carboxylic acid [3-(benzoyl-methyl-amino)-4-phenyl-butyl]-amide;
2,3-Dihydro-benzofuran-5-carboxylic acid [3-(benzoyl-methyl-amino)-4-phenyl-butyl]-amide;
Pyridine-2-carboxylic acid [3-(benzoyl-methyl-amino)-2-hydroxy-4-phenyl-butyl]-amide;
Pyridine-2-carboxylic acid {3-[methyl-(3-methyl-benzoyl)-amino]-4-phenyl-butyl}-amide;
Pyridine-2-carboxylic acid {3-[(3,5-dimethyl-benzoyl)-methyl-amino]-4-phenyl-butyl}-amide;
Pyridine-2-carboxylic acid {3-[(2,6-dimethyl-benzoyl)-methyl-amino]-4-phenyl-butyl}-amide;
Pyridine-2-carboxylic acid {3-[(4-bromo-benzoyl)-methyl-amino]-4-phenyl-butyl}-amide;
Pyridine-2-carboxylic acid {3-[(2-bromo-benzoyl)-methyl-amino]-4-phenyl-butyl}-amide;
Pyridine-2-carboxylic acid {3-[(2,2-dimethyl-benzo[1,3]dioxole-5-carbonyl)-methyl-amino]-4-phenyl-butyl}-amide;
1-Methyl-1H-benzoimidazole-2-carboxylic acid {3-[(3-bromo-benzoyl)-methyl-amino]-4-phenyl-butyl}-amide;
1-Methyl-1H-pyrrole-2-carboxylic acid {3-[(3-methoxy-benzoyl)-methyl-amino]-4-phenyl-butyl}-amide;
1-Methyl-1H-pyrrole-2-carboxylic acid {3-[(3,5-dimethoxy-benzoyl)-methyl-amino]-4-phenyl-butyl}-amide;
1-Methyl-1H-pyrrole-2-carboxylic acid {3-[(3-fluoro-5-methoxy-benzoyl)-methyl-amino]-4-phenyl-butyl}-amide;
1-Methyl-1H-pyrrole-2-carboxylic acid {3-[(3-chloro-5-methoxy-benzoyl)-methyl-amino]-4-phenyl-butyl}-amide;
1-Methyl-1H-pyrrole-2-carboxylic acid {3-[(2,2-difluoro-benzo[1,3]dioxole-5-carbonyl)-methyl-amino]-4-phenyl-butyl}-amide;
Benzooxazole-2-carboxylic acid {3-[(benzo[1,3]dioxole-5-carbonyl)-methyl-amino]-4-phenyl-butyl}-amide;
Benzo[1,3]dioxole-5-carboxylic acid (3-acetylamino-1-benzyl-propyl)-methyl-amide;
Pyridine-2-carboxylic acid {3-[(benzo[1,3]dioxole-5-carbonyl)-methyl-amino]-4,4-dideutero-4-phenyl-butyl}-amide;
Pyridine-2-carboxylic acid [3-(benzoyl-methyl-amino)-3-methyl-4-phenyl-butyl]-amide;
Pyridine-2-carboxylic acid [3-(benzoyl-methyl-amino)-2-methyl-4-phenyl-butyl]-amide;
Pyridine-2-carboxylic acid {3-[(benzo[1,3]dioxole-5-carbonyl)-methyl-amino]-2,2-dimethyl-4-phenyl-butyl}-amide;
Pyridine-2-carboxylic acid [3-(benzoyl-methyl-amino)-4-methyl-4-phenyl-pentyl]-amide;
Pyridine-2-carboxylic acid [3-(benzoyl-methyl-amino)-4-hydroxy-4-phenyl-butyl]-amide;
Pyridine-2-carboxylic acid [3-(benzoyl-methyl-amino)-4-fluoro-4-phenyl-butyl]-amide;
1-Methyl-1H-pyrrole-2-carboxylic acid {3-[(2,3-dihydro-benzofuran-6-carbonyl)-methyl-amino]-4-phenyl-butyl}-amide;
1-Methyl-1H-pyrrole-2-carboxylic acid {3-[(3,5-difluoro-benzoyl)-methyl-amino]-4-phenyl-butyl}-amide;
1-Methyl-1H-pyrrole-2-carboxylic acid {3-[methyl-(3-methyl-benzoyl)-amino]-4-phenyl-butyl}-amide;
1-Methyl-1H-pyrrole-2-carboxylic acid {3-[(3,4-dimethoxy-benzoyl)-methyl-amino]-4-phenyl-butyl}-amide;
1-Methyl-1H-pyrrole-2-carboxylic acid {3-[(3-ethoxy-benzoyl)-methyl-amino]-4-phenyl-butyl}-amide;
1-Methyl-1H-pyrrole-2-carboxylic acid {3-[(3,5-diethoxy-benzoyl)-methyl-amino]-4-phenyl-butyl}-amide;
1-Methyl-1H-pyrrole-2-carboxylic acid {3-[methyl-(3-trifluoromethoxy-benzoyl)-amino]-4-phenyl-butyl}-amide;
1-Methyl-1H-pyrrole-2-carboxylic acid {3-[(3-fluoro-5-methyl-benzoyl)-methyl-amino]-4-phenyl-butyl}-amide;
1-Methyl-1H-pyrrole-2-carboxylic acid {3-[(3,5-dimethyl-benzoyl)-methyl-amino]-4-phenyl-butyl}-amide; and
1-Methyl-1H-pyrrole-2-carboxylic acid {3-[methyl-(3-propoxy-benzoyl)-amino]-4-phenyl-butyl}-amide.

In a further aspect, the invention also provides a process for the production of compounds of the formula I. Compounds of the formula I are obtainable according to the following process as described in scheme 1:

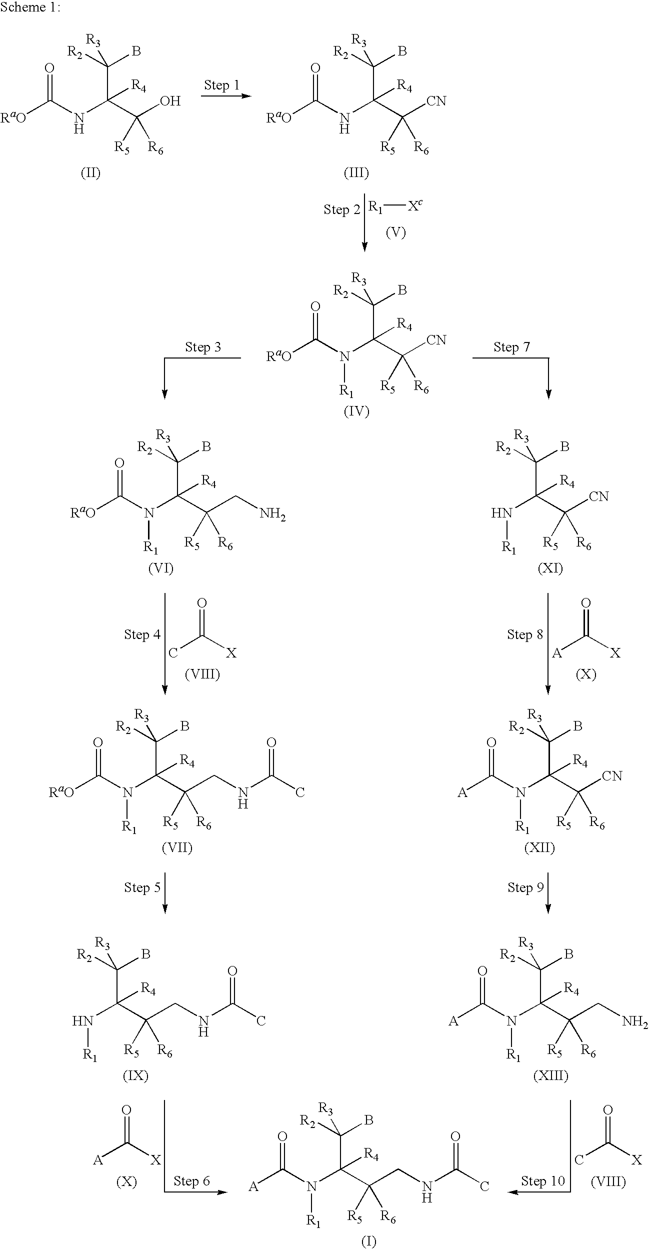

The process steps are described in more detail below:

Step 1: A compound of formula III, in which R₂, R₃, R₄, R₅, R₆ and B are as defined under formula I, and R^{a} is C₁₋₆alkyl, preferably tert-butyl, may be obtained by reacting a compound of formula II, in which R₂, R₃, R₄, R₅, R₆ and B are as defined under formula I, and R^{a} is as defined under formula III, in a first step with methanesulfonyl chloride in the presence of a base, such as triethylamine, in the presence of a suitable solvent, e.g. dichlormethan. The resulting product may then be reacted with sodium cyanide in the presence of a suitable solvent, e.g. dimethylformamide.

Step 2: A compound of formula IV, in which R₁, R₂, R₃, R₄, R₅, R₆ and B are as defined under formula I, and R^{a} is as defined under formula III, may be obtained by reacting the compound of formula III with a compound of formula V, in which R₁ is as defined under formula I, and X^{c} is iodide, in the presence of sodium hydride in the presence of a suitable solvent, e.g. dry tetrahydrofurane.

Step 3: A compound of formula VI, in which R₁, R₂, R₃, R₄, R₅, R₆ and B are as defined under formula I, and R^{a} is as defined under formula III, may be obtained from the compound of formula IV by reduction with e.g. hydrogen/Raney nickel.

Step 4: A compound of formula VII, in which R₁, R₂, R₃, R₄, R₅, R₆, B and C are as defined under formula I, and R^{a} is as defined under formula III, may be obtained by reacting the compound of formula VI with an acid or acid derivative of formula VIII, in which C is defined under formula I, and X is hydroxyl or halogen under suitable reaction conditions as described in the Examples. E.g., when X is halogen in the presence of a suitable base and solvent.

Step 5: A compound of formula IX, in which R₁, R₂, R₃, R₄, R₅, R₆, B and C are as defined under formula I, may be obtained by reacting the compound of formula VII with hydrochloric acid in a suitable solvent, e.g. dichlormethane and dioxane.

Step 6: A compound of formula I, may be obtained by reacting the compound of formula IX with an acid or acid derivative of formula X, in which A is defined under formula I, and X is hydroxyl or halogen as described in step 4.

Step 7: A compound of formula XI, in which R₁, R₂, R₃, R₄, R₅, R₆ and B are as defined under formula I, may be obtained from a compound of formula IV as described in step 5.

Step 8: A compound of formula XII, in which R₁, R₂, R₃, R₄, R₅, R₆ and B are as defined under formula I, may be obtained by reacting the compound of formula XI with a compound of formula X as described in step 6.

Step 9: A compound of formula XIII, in which R₁, R₂, R₃, R₄, R₅, R₆, A and B are as defined under formula I, may be obtained from the compound of formula XII by reduction as described in step 3.

Step 10: A compound of formula I, may be obtained by reacting the compound of formula XIII with a compound of formula VIII as described in step 4.

In a further aspect, the invention also provides a process for the production of compounds of the formula I, in which R₁, R₂, R₃, R₄, R₅, R₆, A, B and C are as defined under formula I, which comprises

reacting a compound of the formula XIII

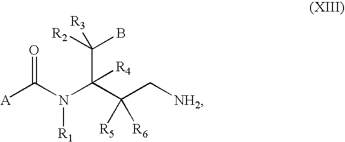

In which R₁, R₂, R₃, R₄, R₅, R₆, A and B are as defined under formula I, with a compound of the formula VIII

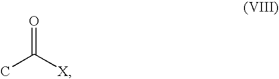

in which C is as defined under formula I, and X is halogen, in the presence of a suitable base and a suitable solvent.

In a further aspect, the invention also provides a process for the production of compounds of the formula I, in which R₁, R₂, R₃, R₄, R₅, R₆, A, B and C are as defined under formula I, which comprises

reacting a compound of the formula IX

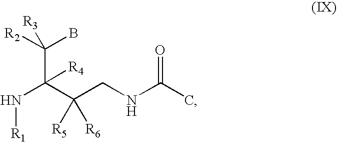

In which R₁, R₂, R₃, R₄, R₅, R₆, B and C are as defined under formula I, with a compound of the formula X

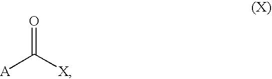

in which A is as defined under formula I, and X is halogen, in the presence of a suitable base and a suitable solvent.

Further compounds of formula I may be obtainable from compounds of formula I—prepared as described according to scheme 1—by reduction, oxidation and/or other functionalization of resulting compounds and/or by cleavage of any protecting group(s) optionally present, and of recovering the so obtainable compound of the formula I.

The reactions can be effected according to conventional methods, for example as described in the Examples.

The work-up of the reaction mixtures and the purification of the compounds thus obtainable may be carried out in accordance with known procedures.

Acid addition salts may be produced from the free bases in known manner, and vice-versa.

Compounds of the formula I can also be prepared by further conventional processes, e.g. as described in the Examples, which processes are further aspects of the invention.

The starting materials of the formulae II, V, VIII and X are known or may be prepared according to conventional procedures starting from known compounds, for example as described in the Examples. In some cases, an intermediate of scheme 1 may be known. In such a situation, said intermediate could be used as an alternative starting point for the process according to scheme 1.

In another aspect, the invention provides a pharmaceutical composition comprising a compound of the invention and a pharmaceutically acceptable carrier. The pharmaceutical composition can be formulated for particular routes of administration such as oral administration, parenteral administration, and rectal administration, etc. In addition, the pharmaceutical compositions of the invention can be made up in a solid form including capsules, tablets, pills, granules, powders or suppositories, or in a liquid form including solutions, suspensions or emulsions. The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers etc.

Typically, the pharmaceutical compositions are tablets and gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with carrier. Carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They are conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The invention further provides anhydrous pharmaceutical compositions and dosage forms comprising the compounds of the invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the invention as an active ingredient will decompose. Such agents, which are referred to herein as “stabilizers,” include, but are not limited to, antioxidants such as ascorbic acid, pH buffers, or salt buffers, etc.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The compounds of formula I in free form or in pharmaceutically acceptable salt form, exhibit valuable pharmacological properties, e.g. orexin receptor modulating properties, e.g. as indicated in in-vitro and in-vivo tests as provided in the next sections and are therefore indicated for therapy.

Compounds of the invention may be useful in the treatment of an indication selected from:

i) sleep disorders;
ii) eating disorders;
iii) substance-related disorders;
iv) Alzheimers disease;
v) psychiatric, neurological and neurodegenerative disorders, such as depression; anxiety; addictions, obsessive compulsive disorder; affective neurosis; depressive neurosis; anxiety neurosis; dysthymic disorder; mood disorder; sexual dysfunction; psychosexual dysfunction; sex disorder; schizophrenia; manic depression; delirium; dementia; severe mental retardation and dyskinesias such as Huntington's disease and Tourette syndrome; Parkinson's disease; ischemic or haemorrhagic stroke; migraine; and neurodegenerative disorder including nosological entities such as disinhibition-dementia-parkinsonism-amyotrophy complex; pallido-punto-nigral degeneration epilepsy; seizure disorders;
vi) cardiovascular diseases, diabetes; asthma; Cushing's syndrome/disease; basophil adenoma; prolactinoma; hyperprolactinemia; hypopituitarism; hypophysis tumour/adenoma; hypothalamic diseases; Froehlich's syndrome; hypophysis diseases, hypothalamic hypogonadism; Kallman's syndrome (anosmia, hyposmia); functional or psychogenic amenorrhea; hypopituitarism; hypothalamic hypothyroidism; hypothalamic-adrenal dysfunction; idiopathic hyperprolactinemia; hypothalamic disorders of growth hormone deficiency; idiopathic growth deficiency; dwarfism; gigantism; acromegaly; heart and lung diseases, acute and congestive heart failure; hypotension; hypertension; urinary retention; osteoporosis; angina pectoris; myocardial infarction; subarachnoid haemorrhage; ulcers; allergies; benign prostatic hypertrophy; chronic renal failure; renal disease; impaired glucose tolerance; vomiting and nausea; inflammatory bowel disease; gastric dyskinesia; gastric ulcers; urinary bladder incontinence e.g. urge incontinence; hyperalgesia; pain; enhanced or exaggerated sensitivity to pain such as hyperalgesia, causalgia, and allodynia; acute pain; burn pain; atypical facial pain; neuropathic pain; back pain; complex regional pain syndrome I and II; arthritic pain; sports injury pain; pain related to infection e.g. HIV, post-chemotherapy pain; post-stroke pain; post-operative pain; neuralgia; conditions associated with visceral pain such as irritable bowel syndrome, migraine and angina; and
vii) other diseases related to general orexin system dysfunction.

Compounds of the invention may be especially useful in the treatment of an indication selected from: sleep disorders, eating disorders, substance-related disorders and Alzheimers disease.

“Eating disorders” may be defined as comprising metabolic dysfunction; dysregulated appetite control; compulsive obesities; emeto-bulimia or anorexia nervosa. This pathologically modified food intake may result from disturbed appetite (attraction or aversion for food); altered energy balance (intake vs expenditure); disturbed perception of food quality (high fat or carbohydrates, high palatability); disturbed food availability (unrestricted diet or deprivation) or disrupted water balance.

“Sleep disorders” include insomnias, narcolepsy and other disorders of excessive sleepiness, sleep-related dystonias; restless leg syndrome; sleep apneas; jet-lag syndrome; shift-work syndrome, delayed or advanced sleep phase syndrome. Insomnias are defined as comprising sleep disorders associated with aging; intermittent treatment of chronic insomnia; situational transient insomnia (new environment, noise) or short-term insomnia due to stress; grief; pain or illness.

“Substance-related disorders” include substance abuse, substance dependence and substance withdrawal disorders, e.g. nicotine withdrawal or narcotics withdrawal.

Thus, as a further embodiment, the invention provides the use of a compound of formula (I) in free form or in pharmaceutically acceptable salt form as a medicament.

As a further embodiment, the invention provides the use of a compound of formula (I) in free form or in pharmaceutically acceptable salt form in therapy.

In a further embodiment, the therapy is selected from a disease which is ameliorated by modulation, preferably antagonism, of orexin receptors. In another embodiment, the disease is selected from the afore-mentioned list, suitably sleep disorders, eating disorders, substance-related disorders or Alzheimers disease.

In another embodiment, the invention provides a method of treating a disease which is ameliorated by modulation, preferably antagonism, of orexin receptors comprising administration of a therapeutically acceptable amount of a compound of formula (I) in free form or in pharmaceutically acceptable salt form. In a further embodiment, the disease is selected from the afore-mentioned list, suitably sleep disorders, eating disorders or Alzheimers disease.

The term “a therapeutically effective amount” of a compound of the invention refers to an amount of the compound of the invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the invention that, when administered to a subject, is effective to (1) at least partially alleviating, inhibiting, preventing and/or ameliorating a condition, or a disorder or a disease (i) mediated by orexin receptors, or (ii) associated with orexin receptor activity, or (iii) characterized by abnormal activity of orexin receptors; or (2) reducing or inhibiting the activity of orexin receptors; or (3) reducing or inhibiting the expression of orexin receptors. In another non-limiting embodiment, the term “a therapeutically effective amount” refers to the amount of the compound of the invention that, when administered to a cell, or a tissue, or a non-cellular biological material, or a medium, is effective to at least partially reducing or inhibiting the activity of orexin receptors; or at least partially reducing or inhibiting the expression of orexin receptors.

As used herein, the term “subject” refers to an animal. Preferably, the animal is a mammal. A subject also refers to for example, primates (e.g., humans), cows, sheep, goats, horses, dogs, cats, rabbits, rats, mice, fish, birds and the like. In a preferred embodiment, the subject is a human.

As used herein, the term “inhibition” or “inhibiting” refers to the reduction or suppression of a given condition, symptom, or disorder, or disease, or a significant decrease in the baseline activity of a biological activity or process.

As used herein, the term “treating” or “treatment” of any disease or disorder refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

The pharmaceutical composition or combination of the invention can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the invention can be applied in vitro in the form of solutions, e.g., preferably aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

The activity of a compound according to the invention can be assessed by in vitro & in vivo methods described herein.

The compound of the invention may be administered either simultaneously with, or before or after, at least one other therapeutic agent. The compound of the invention may be administered separately, by the same or different route of administration, or together in the same pharmaceutical corn position.

The following Examples illustrate the invention, but do not limit it.

### Abbreviations:

| | |
|---|---|
| AcOH | acetic acid |
| DCM | dichloromethane |
| DMF | dimethylformamide |
| DMSO | dimethylsulfoxide |
| EDC | 1-(3-dimethylaminopropyl)-3-ethylcarbodiimide |
| ESIMS | electrospray ionization mass spectrometry |
| EtOAc | ethyl acetate |
| Et₂O | diethyl ether |
| EtOH | ethanol |
| h | hour(s) |
| Hex | hexane |
| HOBt | 1-Hydroxybenzotriazole trihydrate |
| HPLC | high pressure liquid chromatography |
| min | minute(s) |
| NMR | nuclear magnetic resonance spectrometry |
| quant. | quantitative |
| rt | room temperature |
| THF | tetrahydrofurane |
| TFA | trifluoroacetic acid |
| UPLC | ultra performance liquid chromatography |

LCMS/HPLC conditions (%=percent by volume)

### Method A (Rt_{A}=Retention Time A)

Agilent 1100 series, LC-MSD; column Zorbax SB-C18 1.8 μm; 3×30 mm; gradient: A water+0.05% TFA/B acetonitrile+0.05% TFA; 0-3.25 min 70A:30B-0A:100B; 3.25-4.0 min 0A:100B; 4.0-4.25 min 0A:100B-70A:30 B; flow 0.7 ml/min; column temperature 35° C.

### Method B (Rt_{B}=Retention Time B)

Agilent 1100 series, LC-MSD; column Zorbax SB-C18 1.8 μm; 3×30 mm; gradient: A water+0.05% TFA/B acetonitrile+0.05% TFA; 0-3.25 min 90A:10B-0A:100B; 3.25-4.0 min 0A:100B; 4.0-4.25 min 0A:100B-90A:10 B; flow 0.7 ml/min; column temperature 35° C.

### Method C(Rt_{C}=Retention Time C)

Agilent 1100 series, LC-MSD; column Zorbax SB-C18 1.8 μm; 3×30 mm; gradient: A water+0.05% TFA/B acetonitrile+0.05% TFA; 0-3.25 min 60A:40B-0A:100B; 3.25-4.0 min 0A:100B; 4.0-4.25 min 0A:100B-60A:40 B; flow 0.7 ml/min; column temperature 35° C.

### Method D (Rt_{D}=Retention Time D)

Agilent 1100 series, LC-MSD; column Zorbax SB-C18 1.8 μm; 3×30 mm; gradient: A water+0.05% TFA/B acetonitrile+0.05% TFA; 0-3.25 min 100A:0B-0A:100B; 3.25-4.0 min 0A:100B; 4.0-4.25 min 0A:100B-100A:0 B; flow 0.7 ml/min; column temperature 35° C.

### Method E (Rt_{E}=Retention Time E)

Waters Alliance 2690, LC-MSD; column Waters Sunfire C18 2.5 μm; 2.1×50 mm; gradient: A 90% water+10% acetonitrile+0.04% TFA/B 10% water+90% acetonitrile+0.04%

TFA; 0-2.0 min 10A:90B, flow 0.4 ml/min; 2.0-5.0 min 95A:5B, flow 0.4 ml/min; 5.0-6.0 min 10A:90B, flow 0.4 ml/min; column temperature 50° C.

### Method F (Rt_{F}=Retention Time F)

Waters Acquity, HPLC; column Acquity HPLC BEH C18 1.7 μm; 2.1×50 mm; gradient: A 95% water+5% acetonitrile+0.05% FA/B acetonitrile+0.05% FA; 0-2.0 min 95A:5B-0A:100B; 2.0-3.0 min 0A:100B; 3.0-3.1 min 0A: 100B-95A:5 B; 3.1-3.5 min 95A:5B; flow 0.6 ml/min; column temperature 35° C.

### Method G (Rt_{G}=Retention Time G)

Waters Alliance 2690, LC-MSD; column Waters XBridge C18 2.5 μm; 2.1×50 mm; gradient: A acetonitrile+0.1% FA/B water+0.1% FA; 0-2.0 min 10A:90B, flow 0.4 ml/min; 2.0-5.0 min 95A:5B, flow 0.4 ml/min; 5.0-6.0 min 10A:90B, flow 0.4 ml/min; column temperature 50° C.

### Method H(Rt_{H}=Retention Time H)

Waters Acquity UPLC/ZQ2000, column Waters RP Acquity HSS T3 1.8 μm; 2.1×50 mm; gradient: A water+0.05% FA+3.75 mM ammonium acetate/B acetonitrile+0.4% FA; 0-18.4 min 2A:98B, flow 1.0 ml/min; 18.4-20 min 98A:2B, flow 1.0 ml/min; column temperature 50° C.

### Method I (Rt_{I}=Retention Time I)

Waters Acquity HPLC/ZQ2000, column Waters RP Acquity HSS T3 1.8 μm; 2.1×50 mm; gradient: A water+0.05% FA+3.75 mM ammonium acetate/B acetonitrile+0.4% FA; 0-2.15 min 2A:98B, flow 1.2 ml/min; 1.7-2.20 min 98A:2B, flow 1.2 ml/min; column temperature 50° C.

1H-NMR instruments: Varian Mercury (400 MHz); Bruker Advance (600 MHz).

### EXAMPLES

### Example 1

### Pyridine-2-carboxylic acid {(S)-3-[(benzo[1,3]dioxole-5-carbonyl)-methyl-amino]-4-phenyl-butyl}-amide

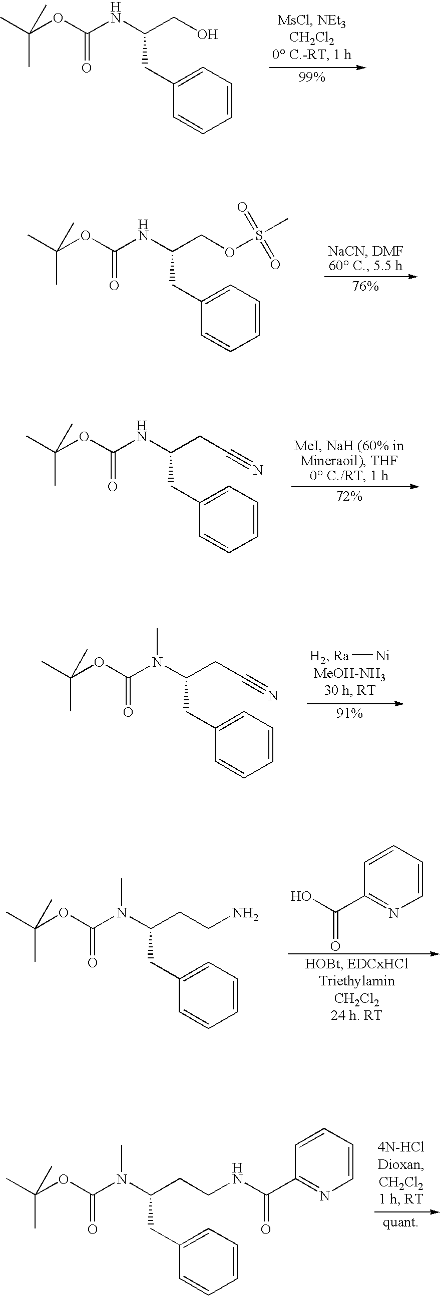

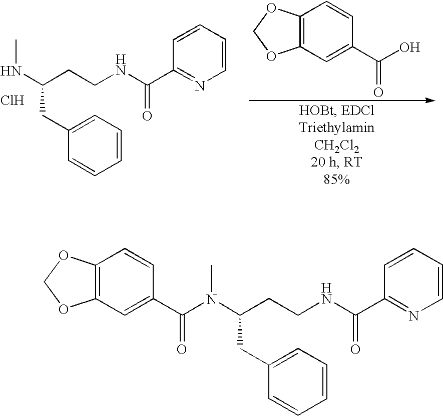

### a) Methanesulfonic acid (S)-2-tert-butoxycarbonylamino-3-phenyl-propyl ester

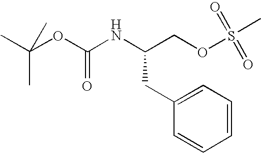

To a solution of ((S)-1-hydroxymethyl-2-phenyl-ethyl)-carbamic acid tert-butyl ester (50.9 g, 202.6 mmol) in dry DCM (800 ml) at 0° C., triethylamine (30.7 g, 303.9 mmol) and methanesulfonyl chloride (34.8 g, 303.9 mol) were added drop wise. The reaction mixture was stirred at 0° C. for 30 min and at rt for another 30 min. Then, the mixture was diluted with EtOAc, washed with 1N—HCl—, NaCl—, NaHCO₃— and NaCl-soln., dried (Na₂SO₄), filtered and concentrated. The crude product was recrystallized from a mixture of DCM-Et₂O-Hex to yield 65.9 g (99%) of the title compound as white solid. [1H-NMR (CDCl₃, 400 MHz) 7.34-7.30 (m, 2H), 7.27-7.21 (m, 3H), 4.72 (br s, 1H), 4.25-4.23 (m, 1H), 4.13-4.09 (m, 2H), 3.01 (s, 3H), 2.93 (dd, 1H), 2.85 (dd, 1H), 1.42 (s, 9H); LCMS Rt_{A}=2.781 min; [M+Na]⁺=352.0].

### b) ((5)-1-Benzyl-2-cyano-ethyl)-carbamic acid tert-butyl ester

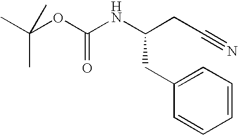

To a solution of methanesulfonic acid (S)-2-tert-butoxycarbonylamino-3-phenyl-propyl ester (65.8 g, 199.7 mmol) in dry DMF (500 ml), sodium cyanide (24.5 g, 499.4 mmol) was added. The reaction mixture was heated at 60° C. for 5 h. Then, the mixture was cooled to 0° C., water was added, the precipitate was filtered off and washed with water. The filtrate was dissolved in EtOAc, washed with NaHCO₃— and NaCl-soln., dried (Na₂SO₄), filtered and concentrated. The crude product was recrystallized from a mixture of DCM-Et₂O-Hex to yield 39.3 g (76%) of the title compound as white solid. [1H-NMR (CDCl₃, 400 MHz) 7.37-7.21 (m, 5H), 4.73 (br d, 1H), 4.08 (br d, 1H), 3.01 (dd, 1H), 2.87 (dd, 1H), 2.71 (dd, 1H), 2.42 (dd, 1H), 1.43 (s, 9H); LCMS Rt_{A}=2.764 min; [M+Na]⁺=283.0].

### c) ((5)-1-Benzyl-2-cyano-ethyl)-methyl-carbamic acid tert-butyl ester

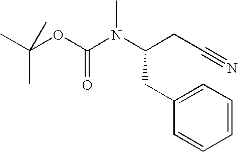

To a solution of ((S)-1-benzyl-2-cyano-ethyl)-carbamic acid tert-butyl ester (6.2 g, 23.8 mmol) in dry THF (55 ml) at 0° C., sodium hydride 60% in mineral oil (2.9 g, 71.4 mmol) was added. Methyl iodide (27.0 g, 191.0 mmol) was added drop wise. The reaction mixture was warmed-up to it and stirred for 1 h. The reaction mixture was exothermic, a water-bath was added to maintain the temperature at 25° C. Then, the mixture was diluted with EtOAc, washed with Na₂S₂O₃—, NaHCO₃— and NaCl-soln., dried (Na₂SO₄), filtered and concentrated. The crude product was purified by chromatography (Flashmaster, Hex to Hex:EtOAc 2:8 over 50 min.) to yield 4.7 g (72%) of the title compound as yellowish oil. [1H-NMR (DMSO, 600 MHz) 7.29-7.25 (m, 2H), 7.21-7.16 (m, 3H), 4.52 (br s, 1H), 2.92 (dd, 1H), 2.83-2.72 (m, 3H), 2.65/2.63 (s, 3H), 1.27/1.18 (s, 9H); LCMS Rt_{A}=3.022 min; [M+Na]⁺=297.0].

### d) ((S)-3-Amino-1-benzyl-propyl)-methyl-carbamic acid tert-butyl ester

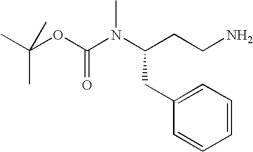

((S)-1-Benzyl-2-cyano-ethyl)-methyl-carbamic acid tert-butyl ester (4.7 g, 17.3 mmol) and Raney-nickel were dissolved in MeOH-5% NH₃ (150 ml) and stirred for 30 h at it under H₂ (1 atm). The reaction mixture was filtered over celite and concentrated. The crude product was purified by chromatography (Flashmaster, DCM to DCM:MeOH-5% NH₃ 85:15 over 50 min.) to yield 4.4 g (91%) of the title compound as yellowish oil. [1H-NMR (DMSO, 600 MHz) 7.26-7.22 (m, 2H), 7.17-7.14 (m, 3H), 4.32 (d, 1H), 2.69 (d, 1H), 2.66 (d, 1H), 2.57/2.56 (s, 3H), 2.49-2.38 (m, 2H), 1.65-1.40 (m, 4H) 1.26/1.16 (s, 9H); LCMS Rt_{B}=2.847 min; [M+H]⁺=279.2].

### e) {(S)-1-Benzyl-3-[(pyridine-2-carbonyl)-amino]-propyl}-methyl-carbamic acid tert-butyl ester

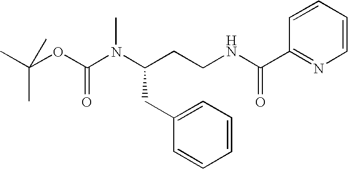

((S)-3-Amino-1-benzyl-propyl)-methyl-carbamic acid tert-butyl ester (1.5 g, 5.4 mmol), picolinic acid (796 mg, 6.5 mmol), HOBt (990 mg, 6.5 mmol), EDC×HCl (1.5 g, 8.1 mmol), and triethylamine (3.0 ml, 21.5 mmol) were dissolved in DCM (80 ml), and the mixture was stirred at it for 24 h. Then, the mixture was diluted with EtOAc, washed with NaHCO₃— and NaCl-soln., dried (Na₂SO₄), filtered and concentrated. The crude product was purified by chromatography (Flashmaster, Hex to Hex:EtOAc 4:6 over 50 min.) to yield 1.9 g (93%) of the title compound as colorless oil. [1H-NMR (DMSO, 600 MHz) 8.76 (d, 1H), 8.62 (s, 1H), 8.04-7.97 (m, 2H), 7.59 (br s, 1H), 7.25-7.23 (m, 2H), 7.15-7.13 (m, 3H), 4.31 (br d, 1H), 3.23-3.09 (m, 2H), 2.78-2.64 (m, 2H), 2.64/2.63 (s, 3H), 1.82-1.65 (m, 2H), 1.27/1.03 (s, 9H); LCMS Rt_{A}=3.147 min; [M+H]⁺=384.2].

### f) Pyridine-2-carboxylic acid ((S)-3-methylamino-4-phenyl-butyl)-amide hydrochloride

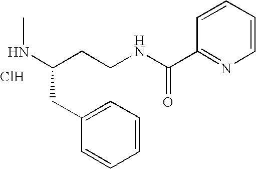

To a solution of {(S)-1-benzyl-3-[(pyridine-2-carbonyl)-amino]-propyl}-methyl-carbamic acid tert-butyl ester (1.9 g, 5.0 mmol) in DCM (10 ml), a 4M HCl solution in dioxane (31.5 ml, 126 mmol) was added drop wise. The reaction mixture was stirred at it for 1 h. Then, the mixture was concentrated, taken-up in DCM and concentrated under high vacuum to yield 1.7 g (quant.) of the title compound as white solid. [1H-NMR (DMSO, 600 MHz) 9.02-8.79 (m, 3H), 8.63 (d, 1H), 8.00-7.98 (m, 2H), 7.62-7.58 (m, 1H), 7.28-7.26 (m, 4H), 7.23-7.19 (m, 1H), 3.42-3.30 (m, 3H), 3.12 (dd, 1H), 2.82 (dd, 1H), 2.60 (t, 3H), 1.84-1.71 (m, 2H); LCMS Rt_{B}=2.465 min; [M+H]⁺=284.2].

### g) Pyridine-2-carboxylic acid {(S)-3-[(benzo[1,3]dioxole-5-carbonyl)-methyl-amino]-4-phenyl-butyl}-amide

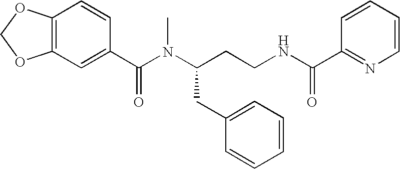

Pyridine-2-carboxylic acid ((S)-3-methylamino-4-phenyl-butyl)-amide hydrochloride (0.5 g, 1.6 mmol), benzo[1,3]dioxole-5-carboxylic acid (312 mg, 1.9 mmol), HOBt (287 mg, 1.9 mmol), EDC×HCl (450 mg, 2.3 mmol) and triethylamine (633 mg, 6.3 mmol) were dissolved in DCM (40 ml) and stirred at rt for 20 h. Then, the mixture was diluted with EtOAc, washed with NaHCO₃— and NaCl-soln., dried (Na₂SO₄), filtered and concentrated. The crude product was purified by chromatography (Flashmaster, Hex:EtOAc 8:2 to Hex:EtOAc 1:9 over 50 min.) to yield 575 mg (85%) of the title compound as white solid. [1H-NMR (DMSO, 600 MHz) 8.68/8.80 (br s, 1H), 8.63-8.61 (m, 1H), 8.03-7.98 (m, 2H), 7.60-7.58 (m, 1H), 7.29-7.19 (m, 4H), 7.05 (d, 1H), 6.88/6.44 (d, 1H), 6.54/6.13 (d, 1H), 6.52/5.91 (s, 1H), 6.02/5.84 (s, 1H), 4.85/3.75 (br s, 1H), 3.40-3.08 (m, 2H), 2.92/2.67 (s, 3H), 2.90-2.80 (m, 2H), 1.98-1.72 (m, 2H); LCMS Rt_{A}=2.505 min; [M+H]⁺=432.2].

### Example 2

### Quinoline-4-carboxylic acid [(S)-3-(benzoyl-methyl-amino)-4-phenyl-butyl]-amide

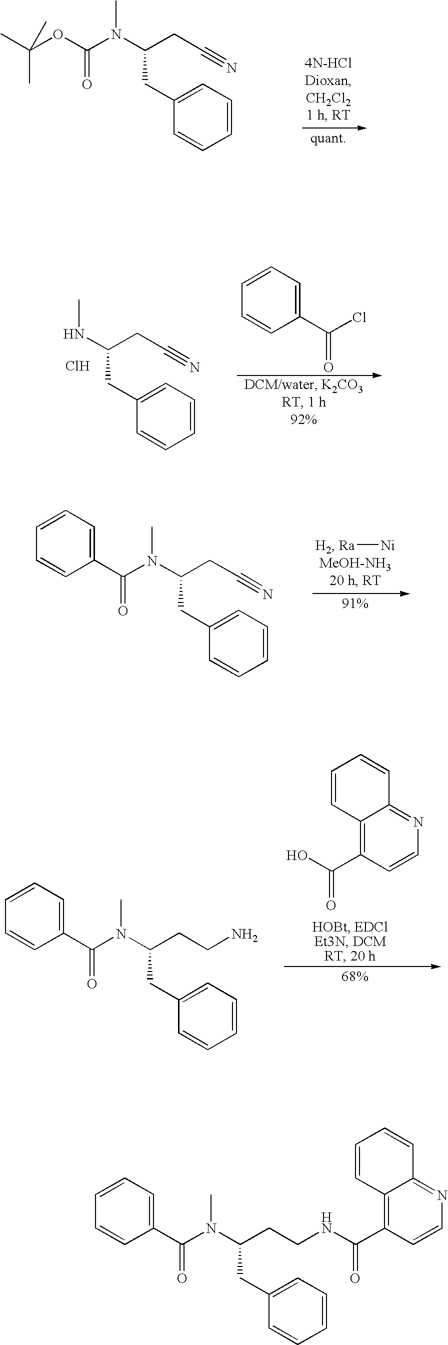

### a) (S)-3-Methylamino-4-phenyl-butyronitrile hydrochloride

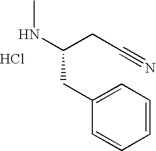

((S)-1-Benzyl-2-cyano-ethyl)-methyl-carbamic acid tert-butyl ester (1.8 g, 6.56 mmol) was dissolved in DCM (10 ml). After the addition of 4M HCl in dioxane (41 ml, 25 eq.) the solution was stirred at it for 45 min. The volatile components were evaporated at reduced pressure to obtain the product as white solid (1.38 g, 6.55 mmol, quant.). The crude product was used for the next step. [1H-NMR (DMSO, 600 MHz) 9.32 (br s, 2H), 7.38-7.29 (m, 5H), 3.76 (dd, 1H), 3.25 (dd, 1H), 2.98 (dd, 1H), 2.87-2.81 (m, 2H), 2.62 (s, 3H); LCMS Rt_{D}=2.465 min; [M+H]⁺=175.2].

### b) N—((S)-1-Benzyl-2-cyano-ethyl)-N-methyl-benzamide

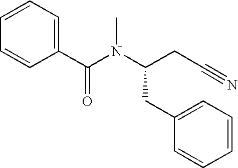

(S)-3-Methylamino-4-phenyl-butyronitrile hydrochloride (2.88 g, 13.67 mmol) was dissolved in DCM (240 ml). Benzoyl chloride (1.74 ml, 15.0 mmol) and potassium carbonate (8.12 g, 58.8 mmol, dissolved in 80 ml water) were added and the mixture was stirred at it for 1 h. EtOAc was added and the organic layer was washed with 0.5 N hydrochloric acid, brine, aqueous sodium bicarbonate, brine, then dried (Na₂SO₄), filtered and concentrated. The crude product was purified by chromatography (Flashmaster, Hex to Hex:EtOAc 2:3 over 40 min.) to yield 3.5 g (92%) of the title compound as yellowish oil. [1H-NMR (DMSO, 600 MHz) 7.38-6.97 (m, 9H), 6.57 (d, 1H), 5.07/4.98 (br s, 1H), 3.13-3.05 (m 1H), 2.98/2.67 (s, 3H), 2.97-2.66 (m, 3H); LCMS Rt_{A}=2.291 min; [M+H]⁺=279.0].

### c) N—((S)-3-Amino-1-benzyl-propyl)-N-methyl-benzamide

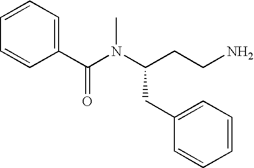

N—((S)-1-Benzyl-2-cyano-ethyl)-N-methyl-benzamide (3.48 g, 12.5 mmol) and Raney-nickel (1.0 g, B113 W Degussa) were dissolved in MeOH-5% NH₃ (100 ml) and mixed for 20 h at it under H₂ (1 atm) in a shaking bottle. The mixture was filtered over celite and concentrated. The crude product was purified by chromatography (Flashmaster, DCM to DCM:MeOH-5% NH₃ 85:15 over 30 min.) to yield 3.2 g (91%) of the title compound as yellow oil. [1H-NMR (DMSO, 600 MHz) 7.35-7.20 (m, 7H), 6.94-6.94 (m, 2H), 6.67 (d, 1H), 4.95/3.67 (br s, 1H), 2.90/2.59 (s, 3H), 2.89-2.29 (m, 4H), 1.96 (br s, 2H), 1.80-1.40 (m, 2H); LCMS Rt_{B}=2.650 min; [M+H]⁺=283.2].

### d) Quinoline-4-carboxylic acid [(S)-3-(benzoyl-methyl-amino)-4-phenyl-butyl]-amide

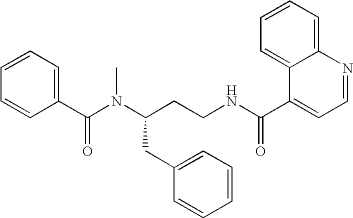

N—((S)-3-Amino-1-benzyl-propyl)-N-methyl-benzamide (100 mg, 0.35 mmol), quinoline-4-carboxylic acid (74 mg, 0.43 mmol), HOBt (65 mg, 0.43 mmol), EDC×HCl (102 mg, 0.53 mmol) and triethylamine (143 mg, 1.42 mmol) were dissolved in DCM (10 ml) and stirred at it for 20 h. The mixture was diluted with EtOAc, washed with NaHCO₃— and NaCl-soln., dried (Na₂SO₄), filtered and concentrated. The crude product was purified by chromatography (Flashmaster, DCM to DCM:MeOH 97:3 over 20 min.) to yield 105 mg (68%) of the title compound as white solid. [1H-NMR (DMSO, 600 MHz) 8.97-8.95 (m, 1H), 8.78/8.72 (t, 1H), 8.17-7.02 (m, 14H), 6.62 (d, 1H), 5.01/3.74 (br s, 1H), 3.42-3.35/3.15-3.10 (m, 2H), 2.98-2.72 (m, 2H), 2.99/2.68 (s, 3H), 2.00-1.77 (m, 2H); LCMS Rt_{B}=3.007 min; [M+H]⁺=438.2]

### Example 3

### 1-Methyl-1H-benzoimidazole-2-carboxylic acid {(S)-3-[(1H-indole-4-carbonyl)-methyl-amino]-4-phenyl-butyl}-amide

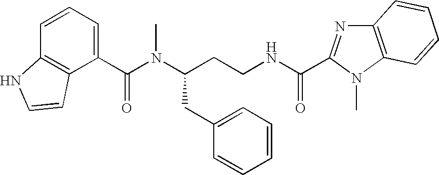

1-Methyl-1H-benzoimidazole-2-carboxylic acid ((S)-3-methylamino-4-phenyl-butyl)-amide hydrochloride (90 mg, 0.22 mmol), 1H-indole-4-carboxylic acid (43 mg, 0.26 mmol), HOBt (40 mg, 0.26 mmol), EDC×HCl (63 mg, 0.33 mmol), and triethylamine (89 mg, 0.88 mmol) were dissolved in DCM (5 ml) and stirred at it for 20 h. Then, the mixture was diluted with EtOAc, washed with NaHCO₃— and NaCl-soln., dried (Na₂SO₄), filtered and concentrated. The crude product was purified by chromatography (Flashmaster, Hex to EtOAc over 45 min.) to yield 50 mg (47%) of the title compound as beige solid. [1H-NMR (DMSO, 600 MHz) 11.20 (d, 1H), 9.06/8.90 (t, 1H), 7.74-7.65 (m, 2H), 7.40-6.39 (m, 11H), 6.17/5.68 (s, 1H), 5.14/3.66 (br s, 1H), 4.13/4.02 (s, 3H), 3.50-3.20 (m, 2H), 3.05/2.60 (s, 3H), 3.0-2.81 (m, 2H), 1.98-1.81/1.53-1.45 (m, 2H); LCMS Rt_{A}=2.824 min; [M+H]⁺=480.2].

### Example 4

### Pyridine-2-carboxylic acid {(S)-3-[methyl-(3-trifluoromethyl-benzoyl)-amino]-4-phenyl-butyl}-amide

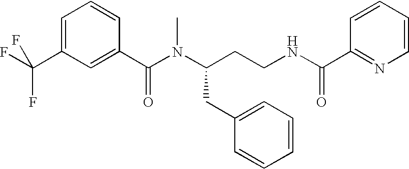

Pyridine-2-carboxylic acid ((S)-3-methylamino-4-phenyl-butyl)-amide hydrochloride (110 mg, 0.33 mmol), 3-trifluoromethyl-benzoic acid (78 mg, 0.41 mmol), HOBt (63 mg, 0.41 mmol), EDC×HCl (99 mg, 0.52 mmol), and triethylamine (139 mg, 1.38 mmol) were dissolved in DCM (8 ml) and stirred at rt for 20 h. Then, the mixture was diluted with EtOAc, washed with NaHCO₃— and NaCl-soln., dried (Na₂SO₄), filtered and concentrated. The crude product was purified by chromatography (Flashmaster, Hex:EtOAc 8:2 to EtOAc over 45 min.) to yield 94 mg (60%) of the title compound as white solid. [1H-NMR (DMSO, 600 MHz) 8.88/8.62 (t, 1H), 8.62 (s, 1H), 8.03-7.94 (m, 2H), 7.74-7.57 (m, 2H), 7.39-6.99 (m, 8H), 7.17/6.53 (s, 1H), 4.91/3.58 (br s, 1H), 3.41-3.34/3.20-3.14 (m, 1H), 2.99/2.63 (s, 3H), 2.96-2.77 (m, 2H), 1.98-1.81 (m, 2H); LCMS Rt_{A}=3.034 min; [M+H]⁺=456.2].

### Example 5

### Pyridine-2-carboxylic acid {(S)-3-[(3,5-bis-trifluoromethyl-benzoyl)-methyl-amino]-4-phenyl-butyl}-amide

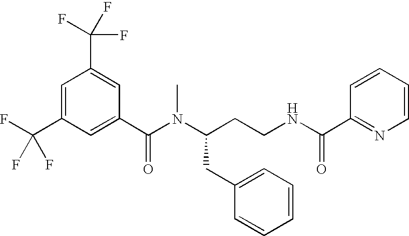

To a solution of pyridine-2-carboxylic acid ((S)-3-methylamino-4-phenyl-butyl)-amide hydrochloride (170 mg, 0.53 mmol) and 3,5-bis-trifluoromethyl-benzoyl chloride (162 mg, 0.59 mmol) in DCM (8 ml), potassium carbonate (316 mg, 2.29 mmol) in 2.5 ml of water was slowly added and the reaction mixture was stirred at it for 1.5 h. Then, the mixture was diluted with EtOAc, washed with NaHCO₃— and NaCl-soln., dried (Na₂SO₄), filtered and concentrated. The crude product was purified by chromatography (Flashmaster, Hex to Hex:EtOAc 3:7 over 35 min.) to yield 0.21 g (75%) of the title compound as white solid. [1H-NMR (DMSO, 600 MHz) 8.88/8.87 (t, 1H), 8.63/8.57 (d, 1H), 8.13/7.89 (s, 1H), 8.02-7.92 (m, 2H), 7.59-7.57 (m, 1H), 7.51/7.26 (s, 1H), 7.30-7.06 (m, 6H), 4.94/3.56 (br s, 1H), 3.43-3.16 (m, 2H), 3.03/2.63 (s, 3H), 2.98-2.80 (m, 2H), 1.99-1.81 (m, 2H); LCMS Rt_{C}=3.087 min; [M+H]⁺=524.0].

### Example 6

### Pyridine-2-carboxylic acid {(S)-3-[methyl-(naphthalene-1-carbonyl)-amino]-4-phenyl-butyl}-amide

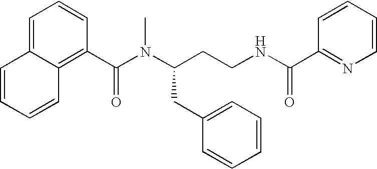

To a solution of pyridine-2-carboxylic acid ((S)-3-methylamino-4-phenyl-butyl)-amide hydrochloride (205 mg, 0.64 mmol) and naphthalene-1-carbonyl chloride (134 mg, 0.71 mmol) in DCM (8 ml), potassium carbonate (381 mg, 2.76 mmol) in 2.5 ml of water was slowly added and the reaction mixture was stirred at it for 1.5 h. Then, the mixture was diluted with EtOAc, washed with NaHCO₃— and NaCl-soln., dried (Na₂SO₄), filtered and concentrated. The crude product was purified by chromatography (Flashmaster, Hex to Hex:EtOAc 2:8 over 45 min.). The fractions corresponding to the desired product were concentrated and recrystallized from a mixture of DCM-Hex-Et₂O to yield 208 mg (74%) of the title compound as white solid. [1H-NMR (DMSO, 600 MHz) 9.02-8.46 (m, 2H), 8.07-6.17 (m, 15H), 5.35-5.15 and 3.55-3.35 (m, 3H), 3.14/3.10 (s, 3H), 3.25-2.65 (m, 2H), 1.95-1.50 (m, 2H); LCMS Rt_{C}=3.034 min; [M+H]⁺=438.2].

### Example 7

### 1H-Indole-5-carboxylic acid {(S)-1-benzyl-3-[(pyridine-2-carbonyl)-amino]-propyl}-methyl-amide

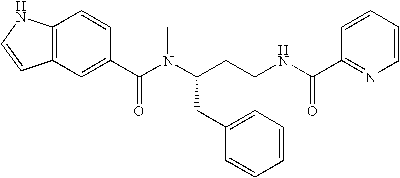

Pyridine-2-carboxylic acid ((S)-3-methylamino-4-phenyl-butyl)-amide hydrochloride (110 mg, 0.34 mmol), 1H-indole-5-carboxylic acid (66 mg, 0.41 mmol), HOBt (63 mg, 0.41 mmol), EDC×HCl (99 mg, 0.52 mmol), and triethylamine (139 mg, 1.38 mmol) were dissolved in DCM (10 ml) and stirred at rt for 20 h. Then, the mixture was diluted with EtOAc, washed with NaHCO₃— and NaCl-soln., dried (Na₂SO₄), filtered and concentrated. The crude product was purified by chromatography (Flashmaster, Hex:EtOAc 8:2 to EtOAc over 30 min.) to yield 100 mg (68%) of the title compound as white solid. [1H-NMR (DMSO, 600 MHz) 11.23/11.11 (s, 1H), 8.93/8.76 (br s, 1H), 8.64/8.56 (s, 1H), 8.04-7.95 (m, 2H), 7.66-7.55 (m, 1H), 7.37-6.69 (m, 8H), 6.81/6.56 (d, 1H), 6.41/6.24 (s, 1H), 4.93/3.85 (br s, 1H), 3.28-2.70 (m, 4H), 2.98/2.73 (s, 3H), 1.96-1.60 (m, 2H); LCMS Rt_{A}=2.339 min; [M+H]⁺=427.2].

### Example 8

### Pyridine-2-carboxylic acid {(S)-3-[(benzo[1,3]dioxole-5-carbonyl)-ethyl-amino]-4-phenyl-butyl}-amide

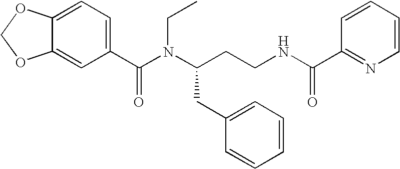

Pyridine-2-carboxylic acid ((S)-3-ethylamino-4-phenyl-butyl)-amide hydrochloride (150 mg, 0.45 mmol), benzo[1,3]dioxole-5-carboxylic acid (90 mg, 0.54 mmol), HOBt (83 mg, 0.54 mmol), EDC×HCl (129 mg, 0.67 mmol), and triethylamine (182 mg, 1.80 mmol) were dissolved in DCM (7.5 ml) and stirred at rt for 4 days. Then, the mixture was diluted with EtOAc, washed with NaHCO₃— and NaCl-soln., dried (Na₂SO₄), filtered and concentrated. The crude product was purified by chromatography (Flashmaster, Hex:EtOAc 8:2 to Hex:EtOAc 1:9 over 25 min.) to yield 143 mg (71%) of the title compound as white solid. [1H-NMR (DMSO, 600 MHz) 8.93/8.80 (s, 1H), 8.62 (s, 1H), 8.04-7.97 (m, 2H), 7.60-7.58 (m, 1H), 7.30-7.00 (m, 5H), 6.87/6.55 (br s, 1H), 6.41/6.24 (d, 1H), 6.02/5.99 (s, 1H), 5.91/5.82 (s, 1H), 3.80-2.77 (m, 7H), 2.08-1.70 (m, 2H), 1.21/0.74 (br s, 3H); LCMS Rt_{A}=2.663 min; [M+H]⁺=446.2].

### Example 9

### Pyridine-2-carboxylic acid {(S)-3-[(3,4-dimethoxy-benzoyl)-methyl-amino]-4-phenyl-butyl}-amide

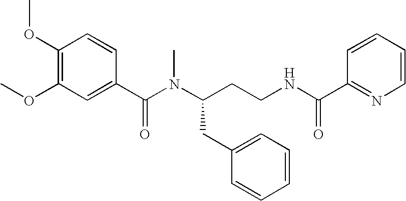

To a solution of pyridine-2-carboxylic acid ((S)-3-methylamino-4-phenyl-butyl)-amide hydrochloride (210 mg, 0.66 mmol) and 3,4-dimethoxy-benzoyl chloride (145 mg, 0.72 mmol) in DCM (8 ml), potassium carbonate (390 mg, 2.82 mmol) in 2.5 ml of water was slowly added and the reaction mixture was stirred at rt for 1.5 h. Then, the mixture was diluted with EtOAc, washed with NaHCO₃— and NaCl-soln., dried (Na₂SO₄), filtered and concentrated. The crude product was purified by chromatography (Flashmaster, Hex:EtOAc 8:2 to EtOAc over 45 min.) to yield 0.19 g (65%) of the title compound as white solid. [1H-NMR (DMSO, 600 MHz) 8.91/8.81 (t, 1H), 8.64-8.60 (m, 1H), 8.03 (d, 1H), 7.98 (br s, 1H), 7.60-7.58 (m, 1H), 7.30-7.19 (m, 4H), 7.02 (d, 1H), 6.90-6.23 (m, 3H), 4.88/3.86 (br s, 1H), 3.73/3.58 (s, 3H), 3.66/3.58 (s, 3H), 3.45-3.11 (m, 2H), 2.93/2.68 (s, 3H), 2.94-2.80 (m, 2H), 1.98-1.70 (m, 2H); LCMS Rt_{A}=2.207 min; [M+H]⁺=448.2].

### Example 10

### Pyridine-2-carboxylic acid {(S)-3-[(3,5-bis-trifluoromethyl-benzoyl)-ethyl-amino]-4-phenyl-butyl}-amide

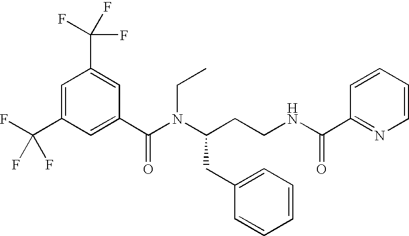

To a solution of pyridine-2-carboxylic acid ((S)-3-ethylamino-4-phenyl-butyl)-amide hydrochloride (150 mg, 0.45 mmol) and 3,5-bis-trifluoromethyl-benzoyl chloride (137 mg, 0.49 mmol) in DCM (8 ml), potassium carbonate (267 mg, 1.93 mmol) in 2.5 ml of water was slowly added and the reaction mixture was stirred at rt for 1.5 h. Then, the mixture was diluted with EtOAc, washed with NaHCO₃— and NaCl-soln., dried (Na₂SO₄), filtered and concentrated. The crude product was purified by chromatography (Flashmaster, Hex to Hex:EtOAc 3:7 over 35 min.) to yield 0.21 g (87%) of the title compound as white solid. [1H-NMR (DMSO, 600 MHz) 8.95/8.76 (t, 1H), 8.64/8.56 (br s, 1H), 8.14/7.89 (s, 1H), 8.04-7.92 (m, 2H), 7.60-7.03 (m, 8H), 3.70-3.05 (m, 5H), 2.87 (d, 2H), 2.10-1.80 (m, 2H), 1.29/0.78 (t, 3H); LCMS Rt_{C}=3.226 min; [M+H]⁺=538.2].

### Example 11

### 1H-Indole-4-carboxylic acid {(S)-1-benzyl-3-[(pyridine-2-carbonyl)-amino]-propyl}-methyl-amide

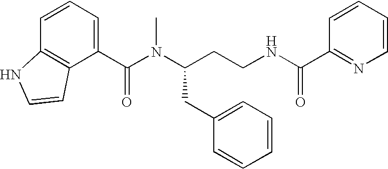

Pyridine-2-carboxylic acid ((S)-3-methylamino-4-phenyl-butyl)-amide hydrochloride (85 mg, 0.27 mmol), 1H-indole-4-carboxylic acid (51 mg, 0.32 mmol), HOBt (49 mg, 0.32 mmol), EDC×HCl (76 mg, 0.40 mmol), and triethylamine (108 mg, 1.06 mmol) were dissolved in DCM (5.0 ml) and stirred at it for 20 h. Then, the mixture was diluted with EtOAc, washed with NaHCO₃— and NaCl-soln., dried (Na₂SO₄), filtered and concentrated. The crude product was purified by chromatography (Flashmaster, Hex to EtOAc over 45 min.) to yield 84 mg (74%) of the title compound as white solid. [1H-NMR (DMSO, 600 MHz) 11.22/11.18 (s, 1H), 8.97/8.64 (t, 1H), 8.64/8.57 (d, 1H), 8.05-7.91 (m, 2H), 7.60-7.54 (m, 1H), 7.37-6.35 (m, 7H), 6.21/5.67 (s, 1H), 5.13/3.64 (br s, 1H), 3.56-3.20 (m, 4H), 3.05/2.59 (s, 3H), 2.99-2.75 (m, 2H), 1.96-1.42 (m, 2H); LCMS Rt_{A}=2.358 min; [M+H]⁺=427.2].

### Example 12

### 1-Methyl-1H-benzoimidazole-2-carboxylic acid {(S)-3-[(3,5-bis-trifluoromethyl-benzoyl)-methyl-amino]-4-phenyl-butyl}-amide

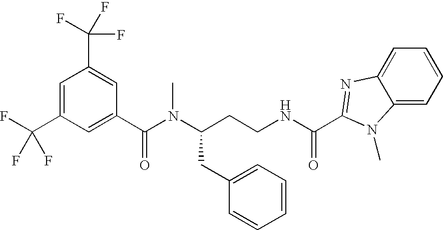

To a solution of 1-methyl-1H-benzoimidazole-2-carboxylic acid ((S)-3-methylamino-4-phenyl-butyl)-amide hydrochloride (100 mg, 0.24 mmol) and 3,5-bis-trifluoromethyl-benzoyl chloride (74 mg, 0.27 mmol) in DCM (8 ml), potassium carbonate (145 mg, 1.05 mmol) in 2.5 ml of water was slowly added and the reaction mixture was stirred at it for 1.5 h. Then, the mixture was diluted with EtOAc, washed with NaHCO₃— and NaCl-soln., dried (Na₂SO₄), filtered and concentrated. The crude product was purified by chromatography (Flashmaster, Hex to Hex:EtOAc 3:7 over 35 min.) to yield 118 mg (84%) of the title compound as white solid. [1H-NMR (DMSO, 600 MHz) 9.02-8.98 (m, 1H), 8.14/7.91 (s, 1H), 7.72-7.65 (m, 2H), 7.56 (s, 1H), 7.38 (dd, 1H), 7.32-7.22 (m, 5H), 7.14 (br s, 1H), 7.08 (br s, 1H), 4.95/3.61 (br s, 1H), 4.09/4.07 (s, 3H), 3.48-3.17 (m, 2H), 3.04/2.65 (s, 3H), 2.97-2.78 (m, 2H), 2.04-1.83 (m, 2H); LCMS Rt_{C}=3.393 min; [M+H]⁺=577.0].

### Example 13

### Pyridine-2-carboxylic acid {(S)-3-[(benzo[1,3]dioxole-5-carbonyl)-propyl-amino]-4-phenyl-butyl}-amide

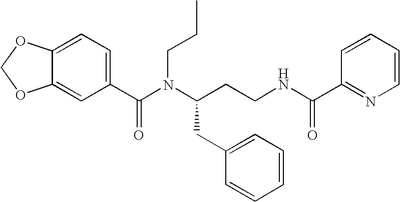

Pyridine-2-carboxylic acid ((S)-4-phenyl-3-propylamino-butyl)-amide_hydrochloride (150 mg, 0.39 mmol), benzo[1,3]dioxole-5-carboxylic acid (78 mg, 0.47 mmol), HOBt (72 mg, 0.47 mmol), EDC×HCl (112 mg, 0.59 mmol), and triethylamine (158 mg, 1.56 mmol) were dissolved in DCM (7.5 ml) and stirred at it for 4 days. Then, the mixture was diluted with EtOAc, washed with NaHCO₃— and NaCl-soln., dried (Na₂SO₄), filtered and concentrated. The crude product was purified by chromatography (Flashmaster, Hex:EtOAc 8:2 to Hex:EtOAc 1:9 over 25 min.) to yield 111 mg (62%) of the title compound as white solid. [1H-NMR (DMSO, 600 MHz) 8.94/8.80 (s, 1H), 8.62 (s, 1H), 8.03-7.97 (m, 2H), 7.59 (dd, 1H), 7.30-7.00 (m, 5H), 6.87/6.53 (br s, 1H), 6.40-5.82 (m, 4H), 3.77 (br s, 1H), 3.45-2.77 (m, 6H), 2.00-1.10 (m, 4H), 0.93/0.48 (br t, 3H); LCMS Rt_{A}=2.929 min; [M+H]⁺=460.2].

### Example 14

### 1H-Indole-2-carboxylic acid {(S)-3-[(benzo[1,3]dioxole-5-carbonyl)-methyl-amino]-4-phenyl-butyl}-amide

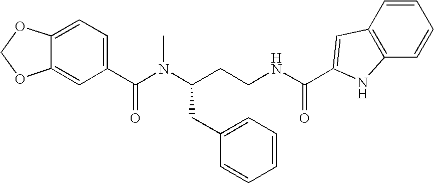

Benzo[1,3]dioxole-5-carboxylic acid ((S)-3-amino-1-benzyl-propyl)-methyl-amide (92 mg, 0.28 mmol), 1H-indole-2-carboxylic acid (55 mg, 0.34 mmol), HOBt (52 mg, 0.34 mmol), EDC×HCl (81 mg, 0.42 mmol), and triethylamine (114 mg, 1.13 mmol) were dissolved in DCM (5 ml) and stirred at rt for 16 h. Then, the mixture was diluted with EtOAc, washed with NaHCO₃— and NaCl-soln., dried (Na₂SO₄), filtered and concentrated. The crude product was purified by chromatography (Flashmaster, Hex to Hex:EtOAc 2:3 over 20 min.) to yield 90 mg (68%) of the title compound as yellowish solid. [1H-NMR (DMSO, 600 MHz) 11.57/11.52 (s, 1H), 8.47/8.41 (br s, 1H), 7.60/7.59 (s, 1H), 7.41/7.40 (s, 1H), 7.30-6.98 (m, 8H), 6.87/6.55 (d, 1H), 6.53/6.18 (d, 1H), 6.53/6.02 (s, 1H), 6.02 (s, 1H), 5.88/5.71 (s, 1H), 4.85/3.80 (br s, 1H), 3.42-3.05 (m, 2H), 2.94/2.69 (s, 3H), 2.96-2.77 (m, 2H), 1.94-1.75 (m, 2H); LCMS Rt_{A}=2.962 min; [M+H]⁺=470.2].

### Example 15

### N—[(S)-3-(Benzoyl-methyl-amino)-4-phenyl-butyl]-benzamide

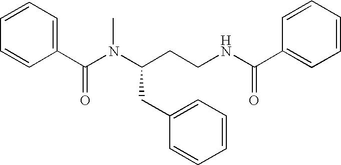

To a solution of N—((S)-3-amino-1-benzyl-propyl)-N-methyl-benzamide (100 mg, 0.35 mmol) and benzoyl chloride (55 mg, 0.39 mmol) in DCM (6 ml), potassium carbonate (210 mg, 1.52 mmol) in 2.1 ml of water was slowly added and the reaction mixture was stirred at it for 1 h. Then, the mixture was diluted with EtOAc, washed with NaHCO₃— and NaCl-soln., dried (Na₂SO₄), filtered and concentrated. The crude product was purified by chromatography (Flashmaster, Hex to Hex:EtOAc 3:7 over 20 min.) to yield 112 mg (82%) of the title compound as beige solid. [1H-NMR (DMSO, 600 MHz) 8.47/8.40 (t, 1H), 7.83/7.74 (d, 2H), 7.53-7.43 (m, 3H), 7.38-6.98 (m, 8H), 6.99/6.59 (d, 2H), 4.92/3.68 (br s, 1H), 3.42-3.03 (m, 2H), 2.96/2.66 (s, 3H), 2.98-2.76 (m, 2H), 1.94-1.71 (m, 2H); LCMS Rt_{A}=2.775 min; [M+H]⁺=387.2].

### Examples 16 to 126

Examples 16-126, as mentioned below, were prepared or can be prepared in analogy to the methods described for Examples 1-15 using the appropriate starting materials or intermediates

### Example 16

### Pyridine-2-carboxylic acid [3-[(3,5-bis-trifluoromethyl-benzoyl)-methyl-amino]-4-(4-chloro-phenyl)-butyl]-amide

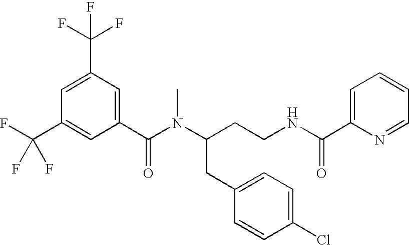

Obtained in analogy to example 2. [LCMS Rt_{A}=3.621 min; [M+H]⁺=558.0; 560.0]

### Example 17

### N-(3-Benzoylamino-1-benzyl-propyl)-N-methyl-3,5-bis-trifluoromethyl-benzamide

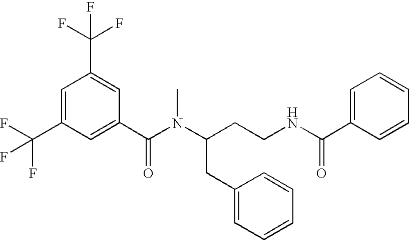

Obtained in analogy to example 1. [LCMS Rt_{A}=3.593 min; [M+H]⁺=523.2]

### Example 18

### 1-Methyl-1H-benzoimidazole-2-carboxylic acid {(S)-3-[methyl-(naphthalene-1-carbonyl)-amino]-4-phenyl-butyl}-amide

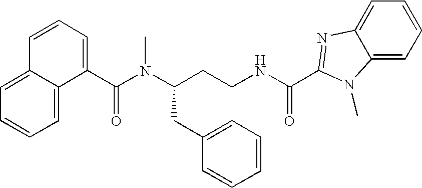

Obtained in analogy to example 12. [LCMS Rt_{C}=2.853 min; [M+H]⁺=491.2]

### Example 19

### 1-Methyl-1H-benzoimidazole-2-carboxylic acid {(8)-3-[(benzo[1,3]dioxole-4-carbonyl)-methyl-amino]-4-phenyl-butyl}-amide

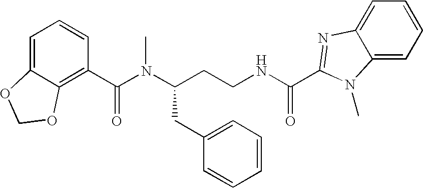

Obtained in analogy to example 3. [LCMS Rt_{A}=2.994 min; [M+H]⁺=485.2]

### Example 20

### Pyridine-2-carboxylic acid {(8)-3-[(benzofuran-5-carbonyl)-methyl-amino]-4-phenyl-butyl}-amide

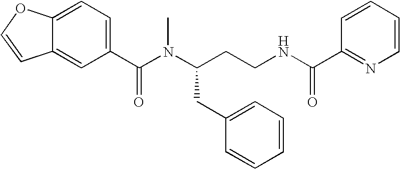

Obtained in analogy to example 4. [LCMS Rt_{A}=2.686 min; [M+H]⁺=428.2]

### Example 21

### Pyridine-2-carboxylic acid {(S)-3-[methyl-(naphthalene-2-carbonyl)-amino]-4-phenyl-butyl}-amide

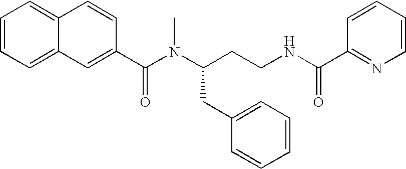

Obtained in analogy to example 6. [LCMS Rt_{C}=2.315 min; [M+H]⁺=438.2]

### Example 22

### Pyridine-2-carboxylic acid {(S)-3-[(3,5-bis-trifluoromethyl-benzoyl)-propyl-amino]-4-phenyl-butyl}-amide

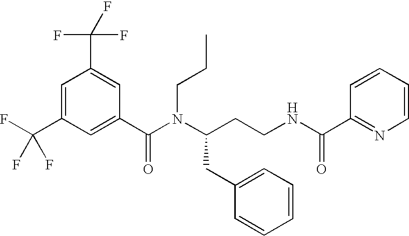

Obtained in analogy to example 6. [LCMS Rt_{C}=3.444 min; [M+H]⁺=552.2]

### Example 23

### 2,3-Dihydro-1H-indole-5-carboxylic acid {(S)-1-benzyl-3-[(pyridine-2-carbonyl)-amino]-propyl}-methyl-amide

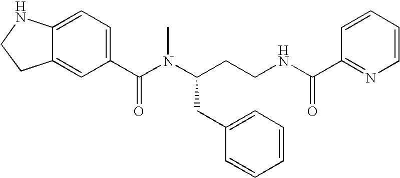

Obtained in analogy to example 4. [LCMS Rt_{B}=2.649 min; [M+H]⁺=429.2]

### Example 24

### 1-Methyl-1H-benzoimidazole-2-carboxylic acid {(S)-4-(4-fluoro-phenyl)-3-[methyl-(naphthalene-1-carbonyl)-amino]-butyl}-amide

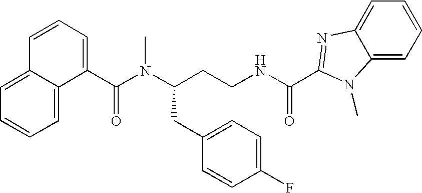

Obtained in analogy to example 12. [LCMS Rt_{C}=2.874 min; [M+H]⁺=509.2]

### Example 25

### Pyridine-2-carboxylic acid [(S)-3-[(benzo[1,3]dioxole-5-carbonyl)-methyl-amino]-4-(4-chloro-phenyl)-butyl]-amide

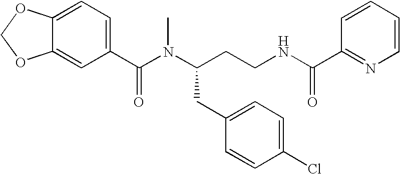

Obtained in analogy to example 6. [LCMS Rt_{A}=2.805 min; [M+H]⁺=466.0]

### Example 26

### 1-Methyl-1H-benzoimidazole-2-carboxylic acid {(S)-4-(4-chloro-phenyl)-3-[methyl-(naphthalene-1-carbonyl)-amino]-butyl}-amide

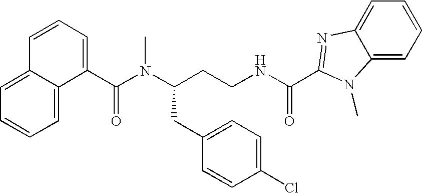

Obtained in analogy to example 12. [LCMS Rt_{C}=3.155 min; [M+H]⁺=525.2]

### Example 27

### 5-(2-Fluoro-phenyl)-2-methyl-thiazole-4-carboxylic acid [(S)-3-(benzoyl-methyl-amino)-4-phenyl-butyl]-amide

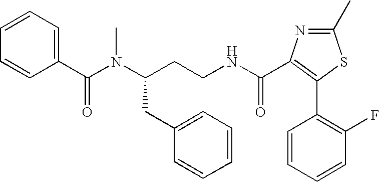

Obtained in analogy to example 2. [LCMS Rt_{C}=2.926 min; [M+H]⁺=502.2]

### Example 28

### Pyridine-2-carboxylic acid {(8)-4-(4-fluoro-phenyl)-3-[methyl-(naphthalene-1-carbonyl)-amino]-butyl}-amide

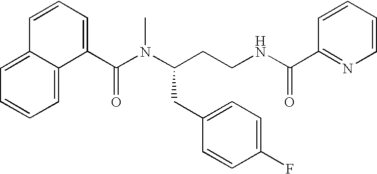

Obtained in analogy to example 6. [LCMS Rt_{C}=2.476 min; [M+H]⁺=456.2]

### Example 29

### Pyridine-2-carboxylic acid {(8)-3-[(2,3-dihydro-benzofuran-5-carbonyl)-methyl-amino]-4-phenyl-butyl}-amide

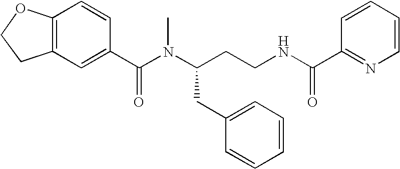

Obtained in analogy to example 4. [LCMS Rt_{A}=2.516 min; [M+H]⁺=430.2]

### Example 30

### Pyridine-2-carboxylic acid {(S)-3-[(benzo[1,3]dioxole-4-carbonyl)-methyl-amino]-4-phenyl-butyl}-amide

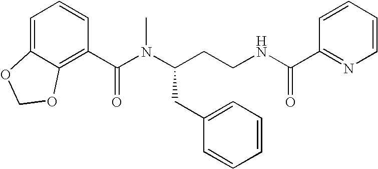

Obtained in analogy to example 4. [LCMS Rt_{A}=2.594 min; [M+H]⁺=432.2]

### Example 31

### 1-Methyl-1H-benzoimidazole-2-carboxylic acid {(S)-3-[(benzo[1,3]dioxole-5-carbonyl)-methyl-amino]-4-phenyl-butyl}-amide

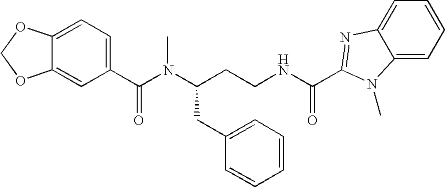

Obtained in analogy to example 3. [LCMS Rt_{A}=2.882 min; [M+H]⁺=485.2]

### Example 32

### Benzo[1,3]dioxole-4-carboxylic acid [(S)-3-(benzoyl-methyl-amino)-4-phenyl-butyl]-amide

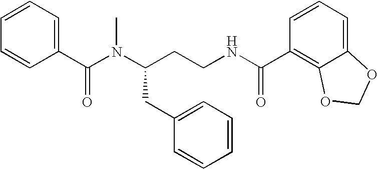

Obtained in analogy to example 2. [LCMS Rt_{A}=2.807 min; [M+H]⁺=431.2]

### Example 33

### 6-Methyl-imidazo[2,1-b]thiazole-5-carboxylic acid [(S)-3-(benzoyl-methyl-amino)-4-phenyl-butyl]-amide

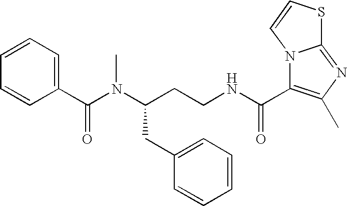

Obtained in analogy to example 2. [LCMS Rt_{B}=2.968 min; [M+H]⁺=447.0]

### Example 34

### Pyridine-2-carboxylic acid {(S)-3-[(3,5-difluoro-benzoyl)-methyl-amino]-4-phenyl-butyl}-amide

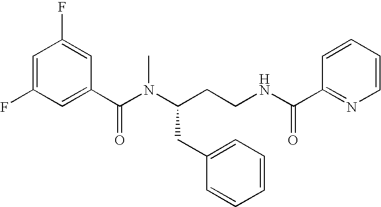

Obtained in analogy to example 4. [LCMS Rt_{A}=2.782 min; [M+H]⁺=424.2]

### Example 35

### Pyridine-2-carboxylic acid {(S)-4-(4-chloro-phenyl)-3-[methyl-(naphthalene-1-carbonyl)-amino]-butyl}-amide

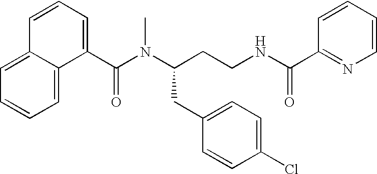

Obtained in analogy to example 6. [LCMS Rt_{C}=2.823 min; [M+H]⁺=472.2]

### Example 36

### 1-Methyl-1H-indole-7-carboxylic acid {(S)-1-benzyl-3-[(pyridine-2-carbonyl)-amino]-propyl}-methyl-amide

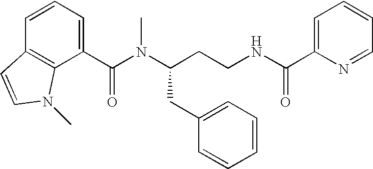

Obtained in analogy to example 4. [LCMS Rt_{A}=3.073 min; [M+H]⁺=441.2]

### Example 37

### Pyridine-2-carboxylic acid [(S)-3-(benzoyl-methyl-amino)-4-phenyl-butyl]-amide

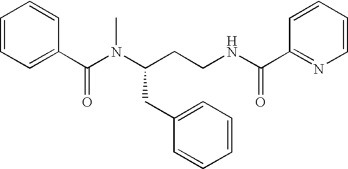

Obtained in analogy to example 6. [LCMS Rt_{A}=2.546 min; [M+H]⁺=388.2]

### Example 38

### 1H-Indole-7-carboxylic acid {(S)-1-benzyl-3-[(pyridine-2-carbonyl)-amino]-propyl}-methyl-amide

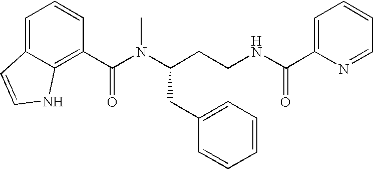

Obtained in analogy to example 4. [LCMS Rt_{A}=2.928 min; [M+H]⁺=427.2]

### Example 39

### N-(1-Benzyl-3-pentanoylamino-propyl)-N-methyl-3,5-bis-trifluoromethyl-benzamide

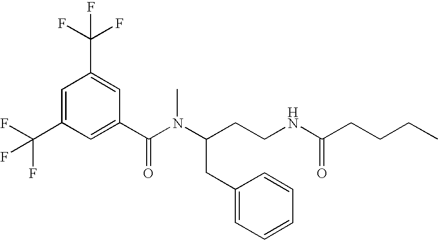

Obtained in analogy to example 2. [LCMS Rt_{A}=3.514 min; [M+H]⁺=503.2]

### Example 40

### 1-Methyl-1H-indole-4-carboxylic acid {(S)-1-benzyl-3-[(pyridine-2-carbonyl)-amino]-propyl}-methyl-amide

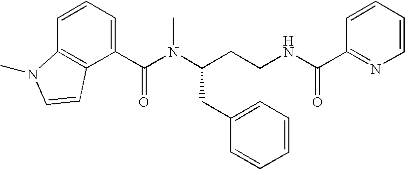

Obtained in analogy to example 4. [LCMS Rt_{A}=2.788 min; [M+H]⁺=441.2]

### Example 41

### Pyridine-2-carboxylic acid {(S)-3-[(3,5-dimethoxy-benzoyl)-methyl-amino]-4-phenyl-butyl}-amide

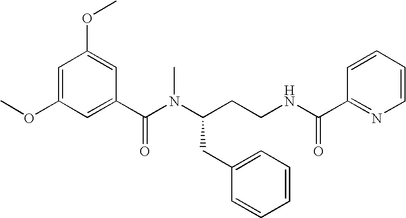

Obtained in analogy to example 6. [LCMS Rt_{A}=2.706 min; [M+H]⁺=448.2]

### Example 42

### Pyridine-2-carboxylic acid {(S)-3-[(4-methoxy-benzoyl)-methyl-amino]-4-phenyl-butyl}-amide

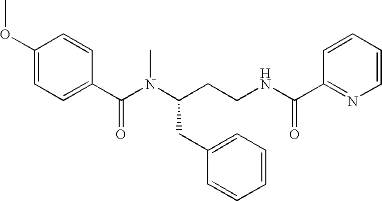

Obtained in analogy to example 6. [LCMS Rt_{A}=2.550 min; [M+H]⁺=418.2]

### Example 43

### Pyridine-2-carboxylic acid {(R)-3-[methyl-(naphthalene-2-carbonyl)-amino]-4-phenyl-butyl}-amide

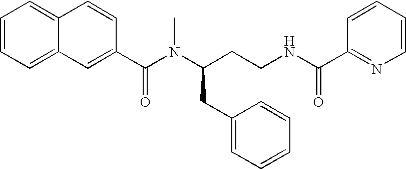

Obtained in analogy to example 6. [LCMS Rt_{A}=2.993 min; [M+H]⁺=438.2]

### Example 44

### 1-Methyl-1H-benzoimidazole-2-carboxylic acid {(S)-3-[methyl-(naphthalene-2-carbonyl)-amino]-4-phenyl-butyl}-amide

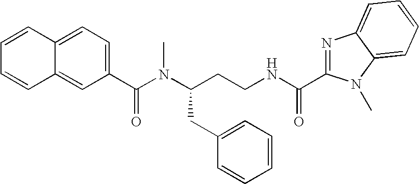

Obtained in analogy to example 12. [LCMS Rt_{C}=2.744 min; [M+H]⁺=491.2]

### Example 45

### Pyridine-2-carboxylic acid {(S)-3-[(benzo[1,3]dioxole-4-carbonyl)-ethyl-amino]-4-phenyl-butyl}-amide

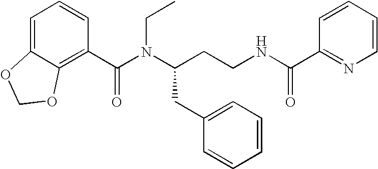

Obtained in analogy to example 4. [LCMS Rt_{A}=2.780 min; [M+H]⁺=446.2]

### Example 46

### Pyridine-2-carboxylic acid {(8)-3-[(2,3-dihydro-benzofuran-7-carbonyl)-methyl-amino]-4-phenyl-butyl}-amide

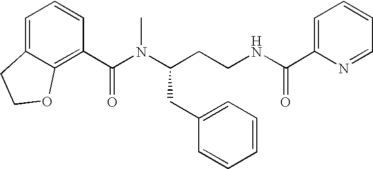

Obtained in analogy to example 6. [LCMS Rt_{A}=2.707 min; [M+H]⁺=430.2]

### Example 47

### Pyridine-2-carboxylic acid {(R)-3-[(benzo[1,3]dioxole-5-carbonyl)-methyl-amino]-4-phenyl-butyl}-amide

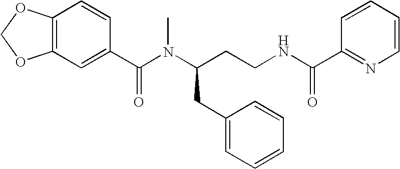

Obtained in analogy to example 6. [LCMS Rt_{A}=2.463 min; [M+H]⁺=432.2]

### Example 48

### 1-Methyl-1H-benzoimidazole-2-carboxylic acid [(S)-3-(benzoyl-methyl-amino)-4-phenyl-butyl]-amide

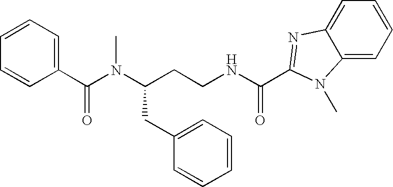

Obtained in analogy to example 12. [LCMS Rt_{C}=2.298 min; [M+H]⁺=441.2]

### Example 49

### Quinoline-8-carboxylic acid [(S)-3-(benzoyl-methyl-amino)-4-phenyl-butyl]-amide

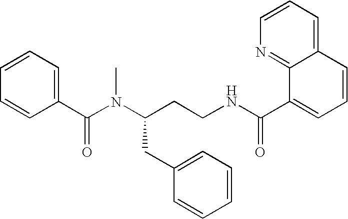

Obtained in analogy to example 2. [LCMS Rt_{B}=3.255 min; [M+H]⁺=438.2]

### Example 50

### 1-Methyl-1H-benzoimidazole-2-carboxylic acid {(S)-4-(4-chloro-phenyl)-3-[methyl-(naphthalene-2-carbonyl)-amino]-butyl}-amide

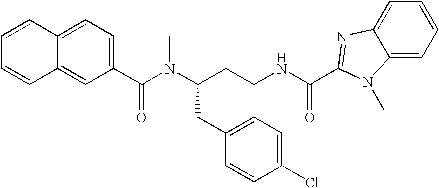

Obtained in analogy to example 12. [LCMS Rt_{C}=2.092 min; [M+H]⁺=525.2]

### Example 51

### Pyridine-2-carboxylic acid [(S)-3-[(benzo[1,3]dioxole-5-carbonyl)-methyl-amino]-4-(4-fluoro-phenyl)-butyl]-amide

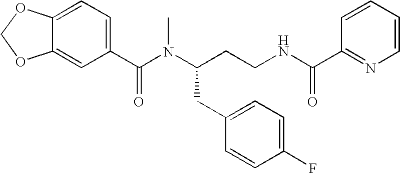

Obtained in analogy to example 4. [LCMS Rt_{A}=2.540 min; [M+H]⁺=450.2]

### Example 52

### Pyridine-2-carboxylic acid {(S)-3-[(3-methoxy-benzoyl)-methyl-amino]-4-phenyl-butyl}-amide

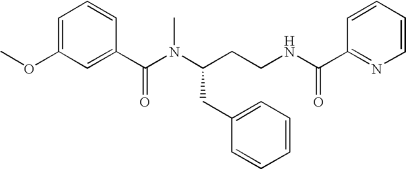

Obtained in analogy to example 6. [LCMS Rt_{A}=2.618 min; [M+H]⁺=418.2]

### Example 53

### Pyridine-2-carboxylic acid {(S)-4-(4-chloro-phenyl)-3-[methyl-(naphthalene-2-carbonyl)-amino]-butyl}-amide

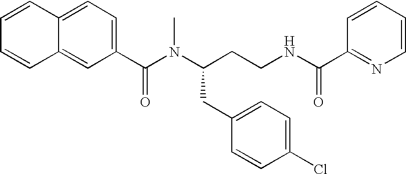

Obtained in analogy to example 6. [LCMS Rt_{C}=2.680 min; [M+H]⁺=472.0]

### Example 54

### Pyridine-2-carboxylic acid {(S)-3-[(2,3-dihydro-benzo[1,4]dioxine-5-carbonyl)-methyl-amino]-4-phenyl-butyl}-amide

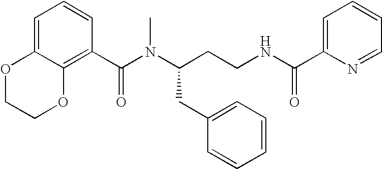

Obtained in analogy to example 4. [LCMS Rt_{A}=2.552/2.766 min (rotamers); [M+H]⁺=446.2]

### Example 55

### 1-Methyl-2,3-dihydro-1H-indole-5-carboxylic acid {(S)-1-benzyl-3-[(pyridine-2-carbonyl)-amino]-propyl}-methyl-amide

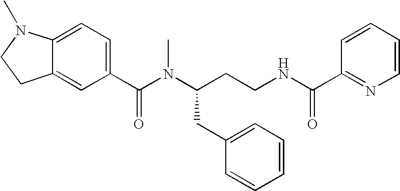

Obtained in analogy to example 4. [LCMS Rt_{A}=2.313 min; [M+H]⁺=443.2]

### Example 56

### 1-Methyl-1H-benzoimidazole-2-carboxylic acid [(S)-3-[(benzo[1,3]dioxole-4-carbonyl)-methyl-amino]-4-(4-fluoro-phenyl)-butyl]-amide

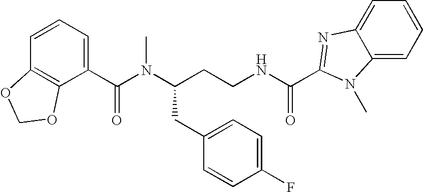

Obtained in analogy to example 3. [LCMS Rt_{C}=2.428 min; [M+H]⁺=503.2]

### Example 57

### 1-Methyl-1H-benzoimidazole-2-carboxylic acid {(S)-4-(4-fluoro-phenyl)-3-[methyl-(naphthalene-2-carbonyl)-amino]-butyl}-amide

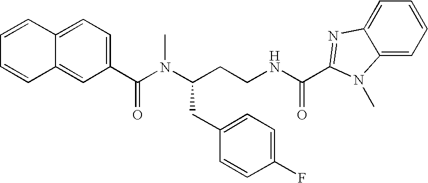

Obtained in analogy to example 12. [LCMS Rt_{C}=2.787 min; [M+H]⁺=509.2]

### Example 58

### Pyridine-2-carboxylic acid {(S)-4-(4-fluoro-phenyl)-3-[methyl-(naphthalene-2-carbonyl)-amino]-butyl}-amide

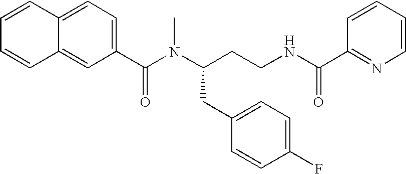

Obtained in analogy to example 6. [LCMS Rt_{C}=2.332 min; [M+H]⁺=456.2]

### Example 59

### Pyridine-2-carboxylic acid {(S)-3-[(benzo[1,3]dioxole-4-carbonyl)-propyl-amino]-4-phenyl-butyl}-amide

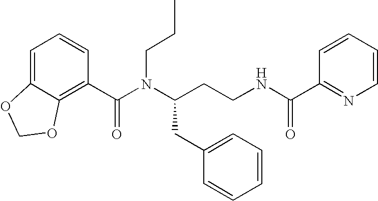

Obtained in analogy to example 4. [LCMS Rt_{A}=3.033 min; [M+H]⁺=460.2]

### Example 60

### 1-Methyl-1H-indole-5-carboxylic acid {3-[(3,5-bis-trifluoromethyl-benzoyl)-methyl-amino]-4-phenyl-butyl}-amide

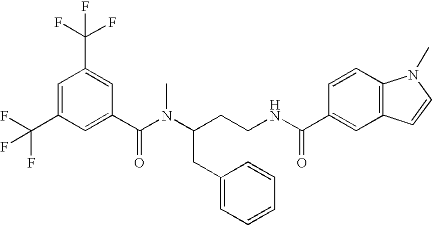

Obtained in analogy to example 2. [LCMS Rt_{A}=3.609 min; [M+H]⁺=576.2]

### Example 61

### Pyridine-2-carboxylic acid {(R)-3-[(benzo[1,3]-dioxole-4-carbonyl)-methyl-amino]-4-phenyl-butyl}-amide

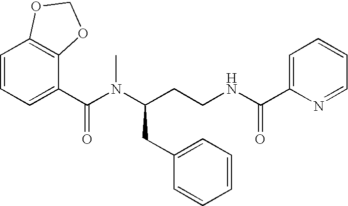

Obtained in analogy to example 6. [LCMS Rt_{A}=2.606 min; [M+H]⁺=432.2]

### Example 62

### Benzo[1,3]dioxole-5-carboxylic acid {(S)-3-[(benzo[1,3]dioxole-5-carbonyl)-amino]-1-benzyl-propyl}-methyl-amide

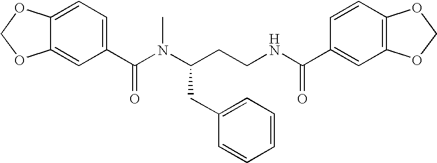

Obtained in analogy to example 14. [LCMS Rt_{A}=2.664 min; [M+H]⁺=475.2]

### Example 63

### Pyridine-2-carboxylic acid [(S)-3-[(benzo[1,3]dioxole-4-carbonyl)-methyl-amino]-4-(4-fluoro-phenyl)-butyl]-amide

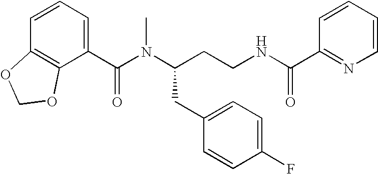

Obtained in analogy to example 4. [LCMS Rt_{A}=2.665 min; [M+H]⁺=450.2]

### Example 64

### 1H-Indole-6-carboxylic acid {(S)-1-benzyl-3-[(pyridine-2-carbonyl)-amino]-propyl}-methyl-amide

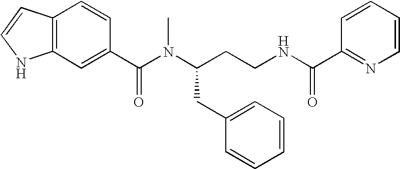

Obtained in analogy to example 4. [LCMS Rt_{A}=2.529 min; [M+H]⁺=427.2]

### Example 65

### Pyridine-2-carboxylic acid [(S)-3-[(benzo[1,3]dioxole-4-carbonyl)-methyl-amino]-4-(4-chloro-phenyl)-butyl]-amide

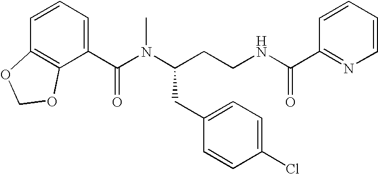

Obtained in analogy to example 4. [LCMS Rt_{A}=2.927 min; [M+H]⁺=466.0]

### Example 66

### Pyridine-2-carboxylic acid [(S)-3-(benzoyl-methyl-amino)-4-(4-fluoro-phenyl)-butyl]-amide

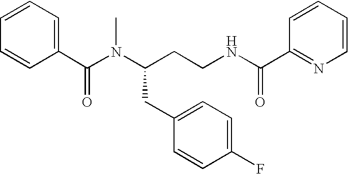

Obtained in analogy to example 6. [LCMS Rt_{A}=2.601 min; [M+H]⁺=406.2]

### Example 67

### 2,3-Dihydro-benzofuran-7-carboxylic acid [(S)-3-(benzoyl-methyl-amino)-4-phenyl-butyl]-amide

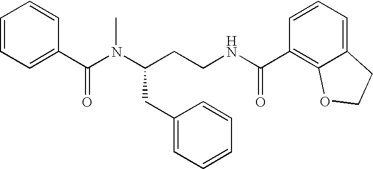

Obtained in analogy to example 2. [LCMS Rt_{A}=2.893 min; [M+H]⁺=429.2]

### Example 68

### Pyridine-2-carboxylic acid [(R)-3-(benzoyl-methyl-amino)-4-phenyl-butyl]-amide

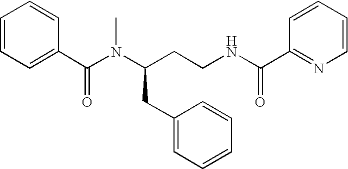

Obtained in analogy to example 6. [LCMS Rt_{A}=2.539 min; [M+H]⁺=388.2]

### Example 69

### Pyridine-2-carboxylic acid [(2R,3S)-3-(benzoyl-methyl-amino)-2-hydroxy-4-phenyl-butyl]-amide

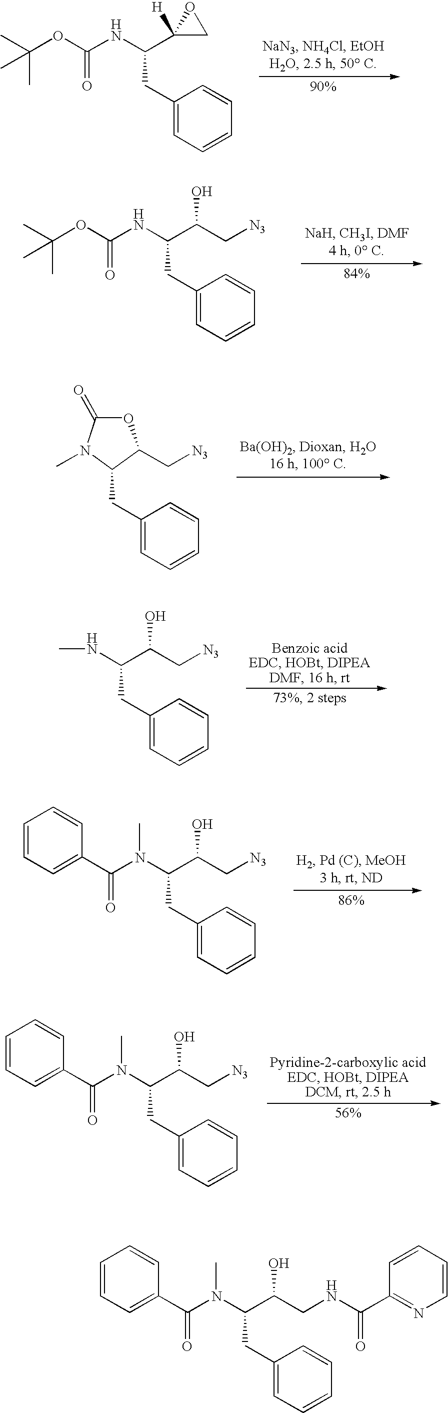

### a) ((1S,2R)-3-Azido-1-benzyl-2-hydroxy-propyl)-carbamic acid tert-butyl ester

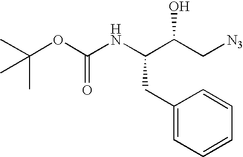

To a solution of ((S)-1-(S)-oxiranyl-2-phenyl-ethyl)-carbamic acid tert-butyl ester (1.0 g, 3.80 mmol) in EtOH (8 ml) and H₂O (2 ml), sodium azide (0.50 g, 7.6 mmol) and ammonium chloride (0.41 g, 7.6 mol) were added. After 1 h at rt, the mixture was heated at 50° C. for 2.5 h. Then, the mixture was cooled to II, and H₂O (10 ml) was added. The white suspension was then 3 times extracted with DCM, the combined organic phases washed with sat. NaCl-soln., dried (Na₂SO₄), filtered and concentrated. This yielded 1.12 g (90%) of the title compound as a white solid which was used for the next step without further purification. [¹H-NMR (D6-DMSO, 400 MHz) 7.19-7.26 (m, 2H), 7.10-7.18 (m, 3H), 6.67 (br d, 1H), 5.46 (d, 1H), 3.47-3.56 (m, 1H), 3.32 (br s, 1H), 3.19 (d, 1H), 2.99 (dd, 1H), 1.23 (s, 9H); LCMS Rt_{A}=2.689 min; [M+Na]⁺=329.2].

### b) (4S,5R)-5-Azidomethyl-4-benzyl-3-methyl-oxazolidin-2-one

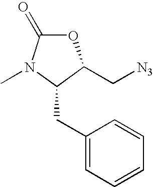

To a solution of ((1S,2R)-3-azido-1-benzyl-2-hydroxy-propyl)-carbamic acid tert-butyl ester (0.5 g, 1.6 mmol) in DMF (10 ml) at 0° C. under nitrogen, sodium hydride (60% in mineral oil, 65 mg, 1.6 mmol) was added. After 4 h, methyl iodide (0.10 ml, 1.63 mmol) dissolved in DMF (1 ml) was added drop wise and then the mixture was warmed to rt. After 30 min. at rt, the reaction mixture was cooled back to 0° C. and quenched with KHSO₄ (5% soln.). The water phase was extracted 3 times with DCM, the combined organic phases dried, filtered and concentrated. The crude product was purified by chromatography (Isolera, Heptane to Heptane:EtOAc 1:1 over 21 min.) to yield 339 mg (84%) of the title compound as clear oil. [¹H-NMR (D6-DMSO, 400 MHz) 7.27-7.34 (m, 4H), 7.19-7.25 (m, 1H), 4.70 (dt, 1H), 4.25 (dd, 1H), 3.66 (dd, 1H), 3.47 (dd, 1H), 3.32 (s, 3H), 2.95 (dd, 1H), 2.88 (dd, 1H); LCMS Rt_{A}=2.151 min; [M+H]⁺=247.0].

### c) (2R,3S)-1-Azido-3-methylamino-4-phenyl-butan-2-ol

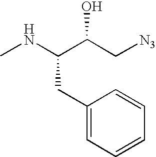

To a solution of (4S,5R)-5-azidomethyl-4-benzyl-3-methyl-oxazolidin-2-one (320 mg, 1.3 mmol) in 1,4-dioxane (6 ml) and H₂O (3 ml) barium hydroxide (492 mg, 2.6 mmol) was added and the reaction mixture was heated at 100° C. for 16 h. The reaction mixture was cooled to rt and the solvents removed in vacuo. The crude mixture of product and barium hydroxide (731 mg) was used directly for the next step. [LCMS Rt_{D}=2.658 min; [M+H]⁺=221.2].

### d) N-((1S,2R)-3-Azido-1-benzyl-2-hydroxy-propyl)-N-methyl-benzamide

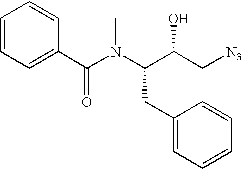

To a solution of (2R,3S)-1-azido-3-methylamino-4-phenyl-butan-2-ol (731 mg, contains Ba(OH)₂, <1.3 mmol), benzoic acid (317 mg, 2.6 mmol), HOBt (412 mg, 2.6 mmol) and Hünig's base (0.89 ml, 5.2 mmol) in DMF (10 ml) was added EDC×HCl (498 mg, 2.6 mmol). After 18 h the reaction mixture was concentrated. The residue was taken up in DCM and 5% NaHCO₃-soln and filtered to remove the precipitated salts. The aqueous phase was extracted 3 times with DCM, the combined organics dried (Na₂SO₄), filtered and concentrated. The crude product was purified by chromatography (Isolera, DCM to DCM:EE 80:20 over 15 min.) to yield 336 mg (73% over 2 steps) of the title compound as white solid. [¹H-NMR (D6-DMSO, 400 MHz, mixture of rotamers) 7.27-7.37 (m, 4H), 7.18-7.27 (m, 3H), 7.10 (t, 1H), 6.97-7.03 (m, 1H), 6.87 (br d, 1H), 6.27-6.34 (br d, 1H), 5.67 (dt, 1H), 3.92-4.05/4.44-4.75 (br s, 1H), 3.42-3.51/3.80-3.89 (m, 1H), 3.30-3.35 (m, 1H), 2.87/2.95 (s, 3H), 3.09-3.26 (m, 1H), 2.78/2.96 (dd, 1H); LCMS Rt_{A}=2.407 min; [M+H]⁺=325.2].

### e) N-((1S,2R)-3-Amino-1-benzyl-2-hydroxy-propyl)-N-methyl-benzamide

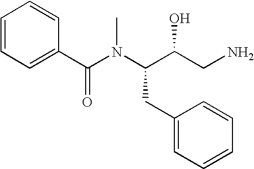

N-((1S,2R)-3-Azido-1-benzyl-2-hydroxy-propyl)-N-methyl-benzamide (335 mg, 1.03 mmol) and 10% of Pd/C (70 mg, 1.03 mmol) were dissolved in MeOH (10 ml) and stirred under H₂-atmosphere (normal pressure) at rt for 5 h. Then, the mixture was filtered and concentrated. The crude product obtained as yellowish oil (291 mg, 86%) was used for the next step. [1H-NMR (DMSO, 400 MHz, mixture of rotamers) 7.19-7.33 (m, 6H), 7.09 (t, 1H), 6.97-7.01 (m, 1H), 6.79-6.84 (m, 1H), 6.28 (d, 1H), 4.62-4.73/4.92-5.05 (m, 1H), 3.46-3.55/3.57-3.67 (m, 1H), 3.25-3.29 (m, 1H), 3.18-3.25/3.36-3.43 (m, 1H), 2.87/2.95 (s, 3H), 2.76/2.95 (dd, 1H), 2.58-2.67 (m, 1H), 2.22-2.32 (m, 1H); LCMS Rt_{D}=2.902 min; [M+H]⁺=299.2].

### f) Pyridine-2-carboxylic acid [(2R,3S)-3-(benzoyl-methyl-amino)-2-hydroxy-4-phenyl-butyl]-amide

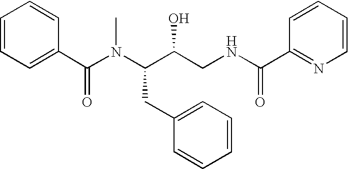

N-((1S,2R)-3-Amino-1-benzyl-2-hydroxy-propyl)-N-methyl-benzamide (200 mg, 0.67 mmol), picolinic acid (91 mg, 0.74 mmol), HOBt (128 mg, 0.80 mmol), EDC×HCl (154 mg, 0.80 mmol) and Hünig's base (0.23 ml, 1.34 mmol) were dissolved in DCM (5 ml) and stirred at rt for 2.5 h. Then, the mixture was diluted with DCM and with NaHCO₃-soln. and the phases separated. The aqueous phases were extracted 2 times with DCM, the combined organic phases dried (Na₂SO₄), filtered and concentrated. The crude product was purified by chromatography (Isolera 1, DCM to DCM:MeOH 95:5 over 30 min.; followed by Isolera 2: DCM:AcOEt 100:0 to 0:100 over 30 min.) to yield 151 mg (56%) of the title compound as white solid. [¹H-NMR (DMSO, 600 MHz, mixture of rotamers) 8.75 (t, 1H), 8.65 (t, 1H), 7.96-8.06 (m, 2H), 7.58-7.63 (m, 1H), 7.27-7.34 (m, 2H), 7.18-7.27/7.29-7.31 (m, 1H), 7.02-7.07/7.25-7.28 (m, 2H), 6.90-6.94/7.31-7.33 (m, 1H), 6.81/7.32 (t, 2H), 6.23/6.86 (br d, 2H), 5.45 (d, 1H), 3.89-3.96 (m, 1H), 3.51-3.57 (m, 1H), 3.04/3.32 (s, 3H), 3.01/3.26 (dd, 1H), 3.00-3.03/3.60-3.65 (m, 1H), 2.83/2.97 (dd, 2H); LCMS Rt_{B}=1.189 min; [M+H]⁺=404.2].

### Example 70

### Pyridine-2-carboxylic acid [(S)-3-(benzoyl-methyl-amino)-4-(4-chloro-phenyl)-butyl]-amide

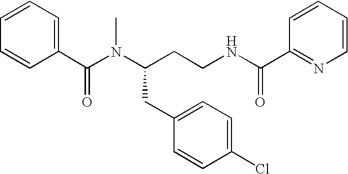

Obtained in analogy to example 5. [LCMS Rt_{A}=2.858 min; [M+H]⁺=422.2]

### Example 71

### Pyridine-2-carboxylic acid {(S)-3-[(2,3-dihydro-benzo[1,4]dioxine-6-carbonyl)-methyl-amino]-4-phenyl-butyl}-amide

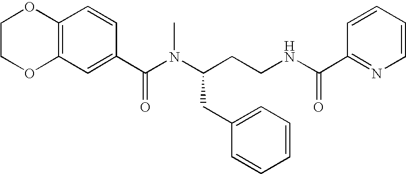

Obtained in analogy to example 4. [LCMS Rt_{A}=2.474 min; [M+H]⁺=446.2]

### Example 72

### Benzo[1,3]dioxole-5-carboxylic acid {(2R,3S)-3-[(benzo[1,3]dioxole-5-carbonyl)-methyl-amino]-2-hydroxy-4-phenyl-butyl}-amide

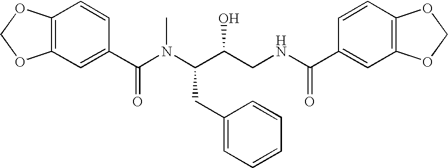

Can be obtained in analogy to example 14 and/or 69. [LCMS Rt_{A}=2.298 min; [M+H]⁺=491.2]

### Example 73

### Pyridine-2-carboxylic acid {(S)-3-[methyl-(4-trifluoromethyl-benzoyl)-amino]-4-phenyl-butyl}-amide

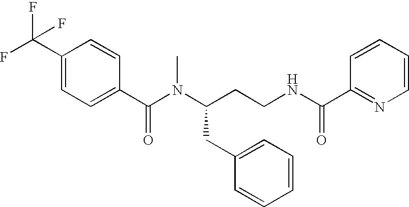

Obtained in analogy to example 4. [LCMS Rt_{A}=3.056 min; [M+H]⁺=456.2].

### Example 74

### Pyridine-2-carboxylic acid {(R)-3-[(3,5-bis-trifluoromethyl-benzoyl)-methyl-amino]-4-phenyl-butyl}-amide

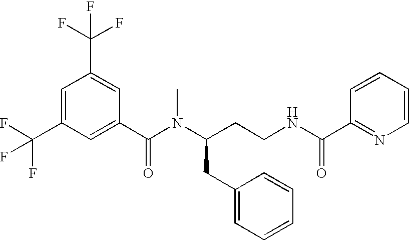

Obtained in analogy to example 6. [LCMS Rt_{A}=3.471 min; [M+H]⁺=524.2]

### Example 75

### 1-Methyl-1H-indole-2-carboxylic acid {(S)-3-[(benzo[1,3]dioxole-5-carbonyl)-methyl-amino]-4-phenyl-butyl}-amide

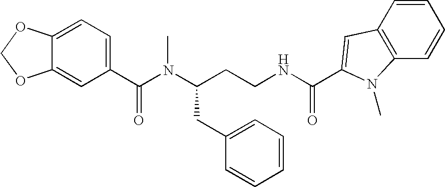

Obtained in analogy to example 14. [LCMS Rt_{A}=3.190 min; [M+H]⁺=484.2].

### Example 76

### 1-Methyl-1H-indole-2-carboxylic acid {(S)-3-[(benzo[1,3]dioxole-5-carbonyl)-methyl-amino]-4-phenyl-butyl}-amide

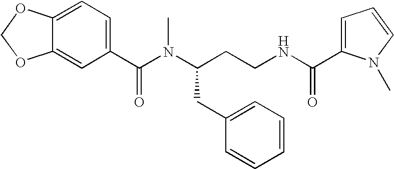

Obtained in analogy to example 14. [LCMS Rt_{A}=2.657 min; [M+H]⁺=434.2].

### Example 77

### Pyridine-2-carboxylic acid {(8)-3-[(3,4-bis-trifluoromethyl-benzoyl)-methyl-amino]-4-phenyl-butyl}-amide

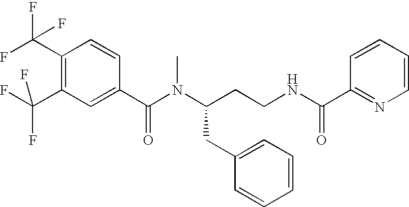

Obtained in analogy to example 4. [LCMS Rt_{C}=2.967 min; [M+H]⁺=524.2]

### Example 78

### 1-Methyl-1H-imidazole-2-carboxylic acid {(S)-3-[(benzo[1,3]dioxole-5-carbonyl)-methyl-amino]-4-phenyl-butyl}-amide

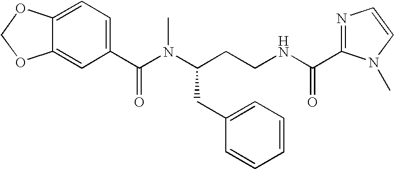

Obtained in analogy to example 4. [LCMS Rt₅=2.923 min; [M+H]⁺=435.2]

### Example 79

### 1-Methyl-1H-benzoimidazole-2-carboxylic acid {(S)-3-[(3-methoxy-benzoyl)-methyl-amino]-4-phenyl-butyl}-amide

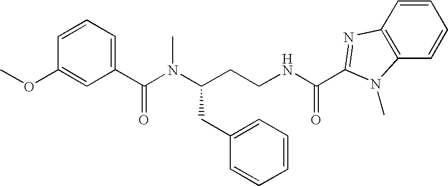

Obtained in analogy to example 4. [LCMS Rt_{A}=3.002 min; [M+H]⁺=471.2]

### Example 80

### 1-Methyl-1H-benzoimidazole-2-carboxylic acid {(S)-3-[(3,4-dimethoxy-benzoyl)-methyl-amino]-4-phenyl-butyl}-amide

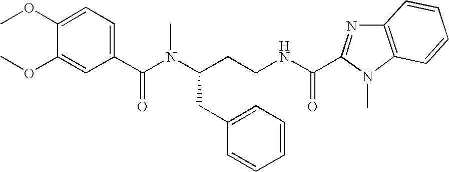

Obtained in analogy to example 4. [LCMS Rt_{A}=2.768 min; [M+H]⁺=501.2]

### Example 81

### 1-Methyl-1H-benzoimidazole-2-carboxylic acid {(S)-3-[(1H-indole-5-carbonyl)-methyl-amino]-4-phenyl-butyl}-amide

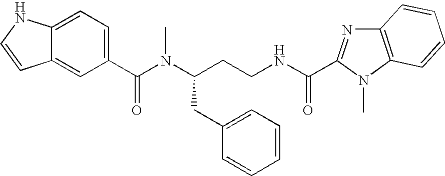

Obtained in analogy to example 3. [LCMS Rt_{A}=2.719 min; [M+H]⁺=480.2]

### Example 82

### 6-Methyl-imidazo[2,1-b]thiazole-5-carboxylic acid {(S)-3-[(benzo[1,3]dioxole-5-carbonyl)-methyl-amino]-4-phenyl-butyl}-amide

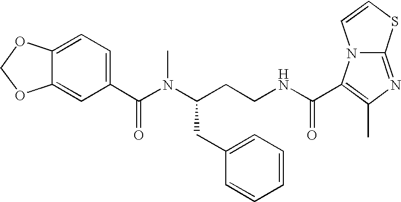

Obtained in analogy to example 14. [LCMS Rt_{B}=2.982 min; [M+H]⁺=491.2]

### Example 83

### 5-(2-Fluoro-phenyl)-2-methyl-thiazole-4-carboxylic acid {(S)-3-[(benzo[1,3]dioxole-5-carbonyl)-methyl-amino]-4-phenyl-butyl}-amide

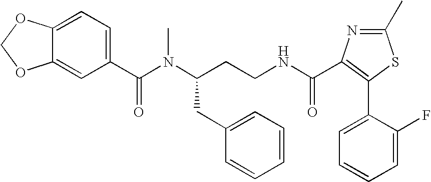

Obtained in analogy to example 14. [LCMS Rt_{A}=3.279 min; [M+H]⁺=546.2]

### Example 84

### 1-Methyl-1H-indole-5-carboxylic acid {(S)-1-benzyl-3-[(pyridine-2-carbonyl)-amino]-propyl}-methyl-amide

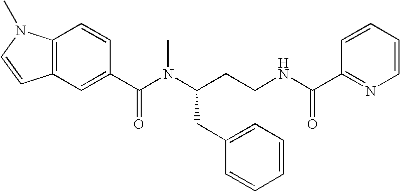

Obtained in analogy to example 4. [LCMS Rt_{A}=2.666 min; [M+H]⁺=441.2]

### Example 85

### 1-Methyl-1H-indole-6-carboxylic acid {(S)-1-benzyl-3-[(pyridine-2-carbonyl)-amino]-propyl}-methyl-amide

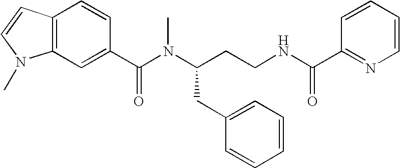

Obtained in analogy to example 4. [LCMS Rt_{A}=2.757 min; [M+H]⁺=441.2]

### Example 86

### N—[(S)-3-(Benzoyl-methyl-amino)-4-phenyl-butyl]-nicotinamide

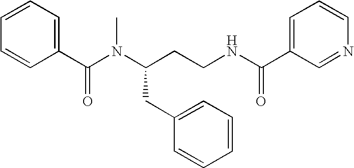

Obtained in analogy to example 2. [LCMS Rt_{B}=2.821 min; [M+H]⁺=388.2]

### Example 87

### Quinoline-2-carboxylic acid [(S)-3-(benzoyl-methyl-amino)-4-phenyl-butyl]-amide

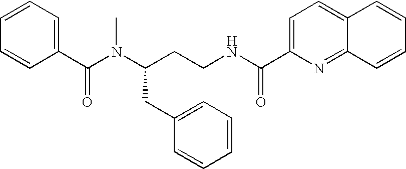

Obtained in analogy to example 2. [LCMS Rt_{A}=3.216 min; [M+H]⁺=438.2]

### Example 88

### Quinoline-7-carboxylic acid [(S)-3-(benzoyl-methyl-amino)-4-phenyl-butyl]-amide

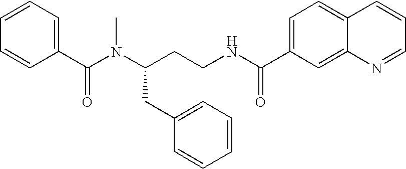

Obtained in analogy to example 2. [LCMS Rt_{B}=2.968 min; [M+H]⁺=438.2]

### Example 89

### Isoquinoline-3-carboxylic acid [(S)-3-(benzoyl-methyl-amino)-4-phenyl-butyl]-amide

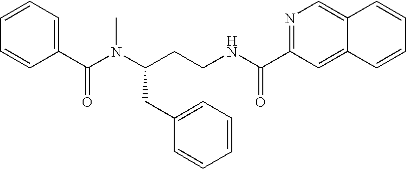

Obtained in analogy to example 2. [LCMS Rt_{A}=3.048 min; [M+H]⁺=438.2]

### Example 90

### 1-Methyl-1H-benzoimidazole-2-carboxylic acid {(S)-3-[(4-methoxy-benzoyl)-methyl-amino]-4-phenyl-butyl}-amide

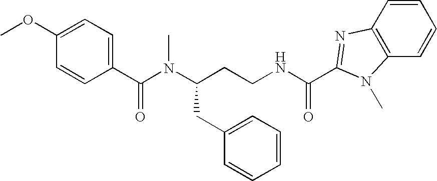

Obtained in analogy to example 12. [LCMS Rt_{A}=2.951 min; [M+H]⁺=471.2]

### Example 91

### 1-Methyl-1H-pyrrole-2-carboxylic acid [(S)-3-(benzoyl-methyl-amino)-4-phenyl-butyl]-amide

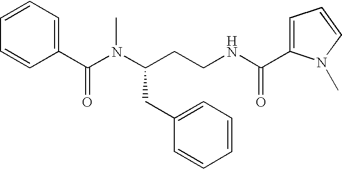

Obtained in analogy to example 2. [LCMS Rt_{A}=2.751 min; [M+H]⁺=390.2]

### Example 92

### 1-Methyl-1H-imidazole-2-carboxylic acid [(S)-3-(benzoyl-methyl-amino)-4-phenyl-butyl]-amide

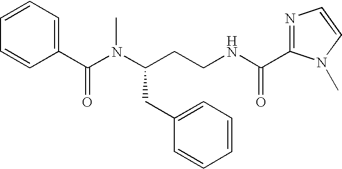

Obtained in analogy to example 2. [LCMS Rt_{B}=2.913 min; [M+H]⁺=391.2]

### Example 93

### Benzo[1,3]dioxole-5-carboxylic acid [(S)-3-(benzoyl-methyl-amino)-4-phenyl-butyl]-amide

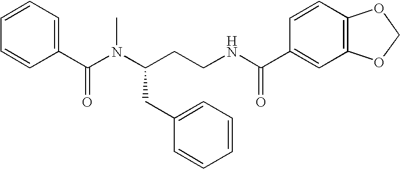

Obtained in analogy to example 2. [LCMS Rt_{A}=2.758 min; [M+H]⁺=431.2]

### Example 94

### 2,3-Dihydro-benzofuran-5-carboxylic acid [(S)-3-(benzoyl-methyl-amino)-4-phenyl-butyl]-amide

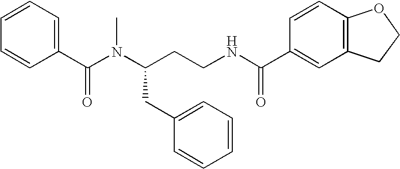

Obtained in analogy to example 2. [LCMS Rt_{A}=2.762 min; [M+H]⁺=429.2]

### Example 95

### Pyridine-2-carboxylic acid [(2S,3S)-3-(benzoyl-methyl-amino)-2-hydroxy-4-phenyl-butyl]-amide

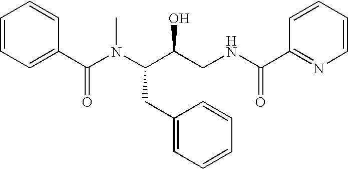

Obtained in analogy to example 69, starting from ((5)-1-(R)-oxiranyl-2-phenyl-ethyl)-carbamic acid tert-butyl ester. [LCMS Rt_{A}=2.175 min; [M+H]⁺=404.2]

### Example 96

### Pyridine-2-carboxylic acid {(S)-3-[methyl-(3-methyl-benzoyl)-amino]-4-phenyl-butyl}-amide

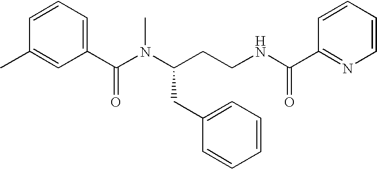

Obtained in analogy to example 4. [LCMS Rt_{A}=2.830 min; [M+H]⁺=402.2]

### Example 97

### Pyridine-2-carboxylic acid {(S)-3-[(3,5-dimethyl-benzoyl)-methyl-amino]-4-phenyl-butyl}-amide

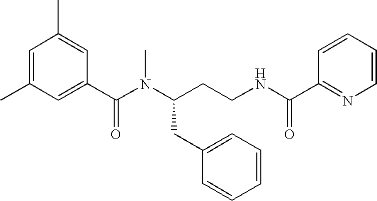

Obtained in analogy to example 4. [LCMS Rt_{A}=3.010 min; [M+H]⁺=416.2]

### Example 98

### Pyridine-2-carboxylic acid {(8)-3-[(2,6-dimethyl-benzoyl)-methyl-amino]-4-phenyl-butyl}-amide

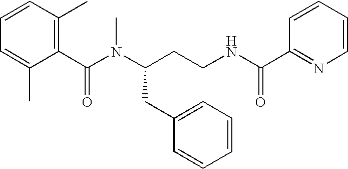

Obtained in analogy to example 6. [LCMS Rt_{A}=3.126 min; [M+H]⁺=416.2]

### Example 99

### Pyridine-2-carboxylic acid {(S)-3-[(4-bromo-benzoyl)-methyl-amino]-4-phenyl-butyl}-amide

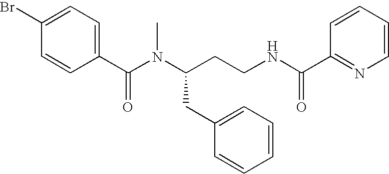

Obtained in analogy to example 4. [LCMS Rt_{A}=2.953 min; [M+H]⁺=466.01468.0]

### Example 100

### Pyridine-2-carboxylic acid {(8)-3-[(2-bromo-benzoyl)-methyl-amino]-4-phenyl-butyl}-amide

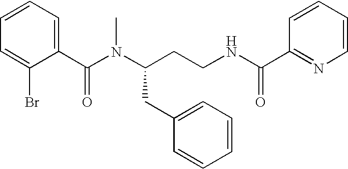

Obtained in analogy to example 4. [LCMS Rt_{A}=2.994 min; [M+H]⁺=466.01468.0]

### Example 101

### Pyridine-2-carboxylic acid {(S)-3-[(2,2-dimethyl-benzo[1,3]dioxole-5-carbonyl)-methyl-amino]-4-phenyl-butyl}-amide

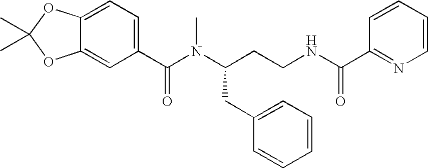

Obtained in analogy to example 4. [LCMS Rt_{A}=2.901 min; [M+H]⁺=460.2]

### Example 102

### 1-Methyl-1H-benzoimidazole-2-carboxylic acid {(S)-3-[(3-bromo-benzoyl)-methyl-amino]-4-phenyl-butyl}-amide

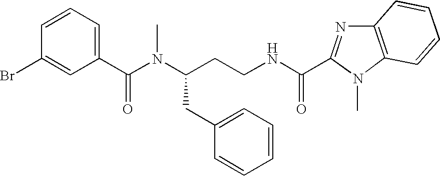

Obtained in analogy to example 3. [LCMS Rt_{A}=3.305 min; [M+H]⁺=519.0/521.0]

### Example 103

### 1-Methyl-1H-pyrrole-2-carboxylic acid {(S)-3-[(3-methoxy-benzoyl)-methyl-amino]-4-phenyl-butyl}-amide

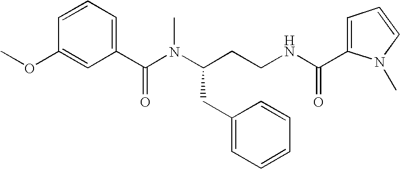

Obtained in analogy to example 4. [LCMS Rt_{F}=1.18 min; [M+H]⁺=420.3]

### Example 104

### 1-Methyl-1H-pyrrole-2-carboxylic acid {(S)-3-[(3,5-dimethoxy-benzoyl)-methyl-amino]-4-phenyl-butyl}-amide

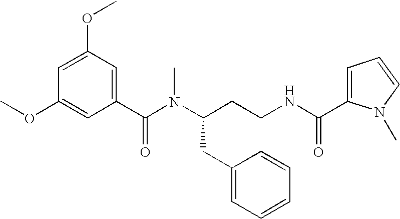

Obtained in analogy to example 4. [LCMS Rt_{C}=2.175 min; [M+H]⁺=450.2]

### Example 105

### 1-Methyl-1H-pyrrole-2-carboxylic acid {(S)-3-[(3-fluoro-5-methoxy-benzoyl)-methyl-amino]-4-phenyl-butyl}-amide

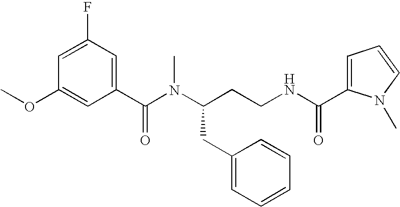

Obtained in analogy to example 4. [LCMS Rt_{A}=2.998 min; [M+H]⁺=438.2]

### Example 106

### 1-Methyl-1H-pyrrole-2-carboxylic acid {(S)-3-[(3-chloro-5-methoxy-benzoyl)-methyl-amino]-4-phenyl-butyl}-amide

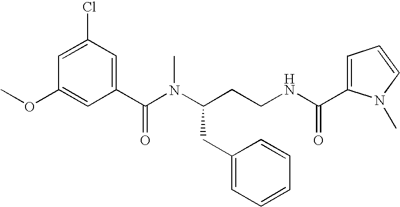

Obtained in analogy to example 4. [LCMS Rt_{C}=2.631 min; [M+H]⁺=454.2]

### Example 107

### 1-Methyl-1H-pyrrole-2-carboxylic acid {(S)-3-[(2,2-difluoro-benzo[1,3]dioxole-5-carbonyl)-methyl-amino]-4-phenyl-butyl}-amide

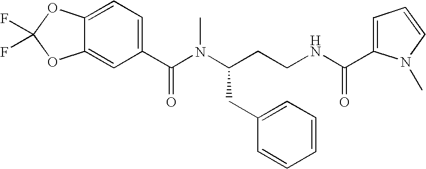

Obtained in analogy to example 4. [LCMS Rt_{A}=3.171 min; [M+H]⁺=470.2]

### Example 108

### Benzooxazole-2-carboxylic acid {(S)-3-[(benzo[1,3]dioxole-5-carbonyl)-methyl-amino]-4-phenyl-butyl}-amide

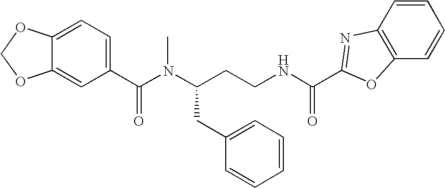

Obtained in analogy to example 14. [LCMS Rt_{A}=2.880 min; [M+H]⁺=472.0]

### Example 109

### Benzo[1,3]-dioxole-5-carboxylic acid ((S)-3-acetylamino-1-benzyl-propyl)-methyl-amide

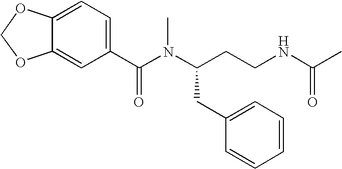

Obtained in analogy to example 14. [LCMS Rt_{B}=2.943 min; [M+H]⁺=369.2]

### Example 110

### Pyridine-2-carboxylic acid {3-[(benzo[1,3]dioxole-5-carbonyl)-methyl-amino]-4,4-dideutero-4-phenyl-butyl}-amide

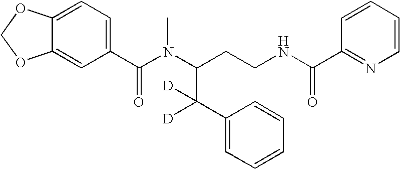

Obtained in analogy to example 1, starting from dideutero-phenylalanine. [LCMS Rt_{G}=2.54 min; [M+H]⁺=434.3]

### Example 111

### Pyridine-2-carboxylic acid [(S)-3-(benzoyl-methyl-amino)-3-methyl-4-phenyl-butyl]-amide

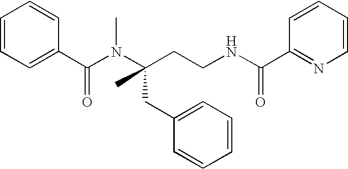

[LCMS Rt_{H}=7.49 min; [M+H]⁺=402]

### Example 112

### Pyridine-2-carboxylic acid [(2R,3S)-3-(benzoyl-methyl-amino)-2-methyl-4-phenyl-butyl]-amide

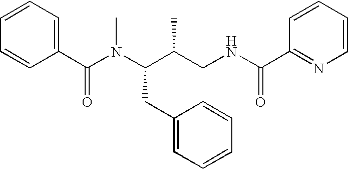

[LCMS Rt_{G}=2.00 min; [M+H]⁺=402.3]

### Example 113

### Pyridine-2-carboxylic acid {(S)-3-[(benzo[1,3]dioxole-5-carbonyl)-methyl-amino]-2,2-dimethyl-4-phenyl-butyl}-amide

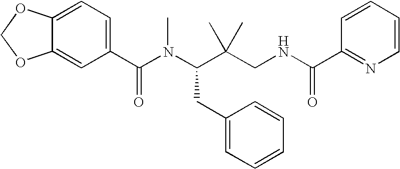

[LCMS Rt_{G}=2.00 min; [M+H]⁺=460.3]

### Example 114

### Pyridine-2-carboxylic acid [(R)-3-(benzoyl-methyl-amino)-4-methyl-4-phenyl-pentyl]-amide

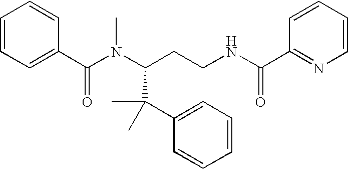

[LCMS Rt_{H}=7.78 min; [M+H]⁺=416]

### Example 115

### Pyridine-2-carboxylic acid [(3R,4S)-3-(benzoyl-methyl-amino)-4-hydroxy-4-phenyl-butyl]-amide

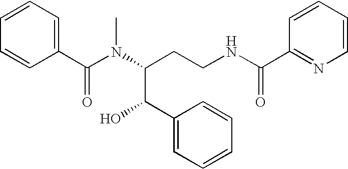

[LCMS Rt_{I}=1.58 min; [M+H]⁺=404.2]

### Example 116

### Pyridine-2-carboxylic acid [(3R,4S)-3-(benzoyl-methyl-amino)-4-fluoro-4-phenyl-butyl]-amide

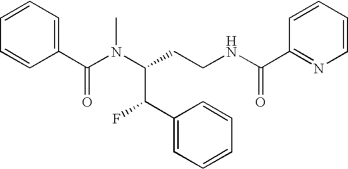

[LCMS Rt_{I}=1.18 min; [M+H]⁺=406.4]

### Example 117

### 1-Methyl-1H-pyrrole-2-carboxylic acid {(S)-3-[(2,3-dihydro-benzofuran-6-carbonyl)-methyl-amino]-4-phenyl-butyl}-amide

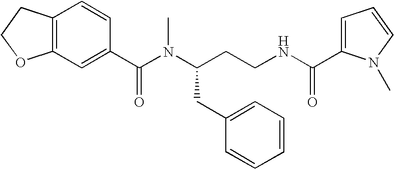

Obtained in analogy to example 4. [LCMS Rt_{A}=2.712 min; [M+H]⁺=432.2]

### Example 118

### 1-Methyl-1H-pyrrole-2-carboxylic acid {(8)-3-[(3,5-difluoro-benzoyl)-methyl-amino]-4-phenyl-butyl}-amide

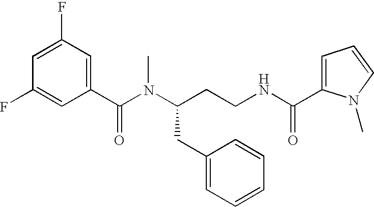

Obtained in analogy to example 4. [LCMS Rt_{F}=1.22 min; [M+H]⁺=404.3]

### Example 119

### 1-Methyl-1H-pyrrole-2-carboxylic acid {(8)-3-[methyl-(3-methyl-benzoyl)-amino]-4-phenyl-butyl}-amide

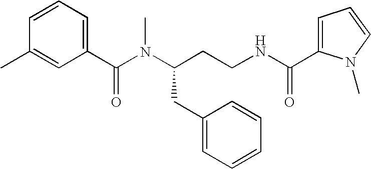

Obtained in analogy to example 4. [LCMS Rt_{F}=1.22 min; [M+H]⁺=404.3]

### Example 120

### 1-Methyl-1H-pyrrole-2-carboxylic acid {(8)-3-[(3,4-dimethoxy-benzoyl)-methyl-amino]-4-phenyl-butyl}-amide

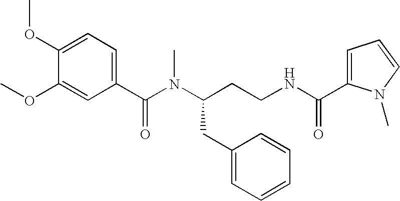

Obtained in analogy to example 4. [LCMS Rt_{F}=0.97 min; [M+H]⁺=450.3]

### Example 121

### 1-Methyl-1H-pyrrole-2-carboxylic acid {(S)-3-[(3-ethoxy-benzoyl)-methyl-amino]-4-phenyl-butyl}-amide

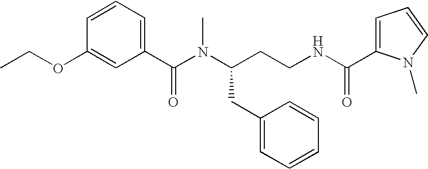

Obtained in analogy to example 4. [LCMS Rt_{F}=1.25 min; [M+H]⁺=434.3]

### Example 122

### 1-Methyl-1H-pyrrole-2-carboxylic acid {(S)-3-[(3,5-diethoxy-benzoyl)-methyl-amino]-4-phenyl-butyl}-amide

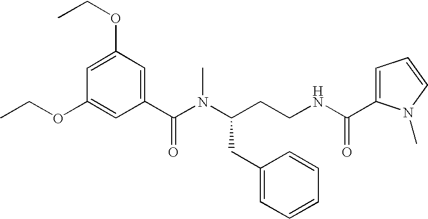

Obtained in analogy to example 4. [LCMS Rt_{F}=1.34 min; [M+H]⁺=478.3]

### Example 123

### 1-Methyl-1H-pyrrole-2-carboxylic acid {(S)-3-[methyl-(3-trifluoromethoxy-benzoyl)-amino]-4-phenyl-butyl}-amide

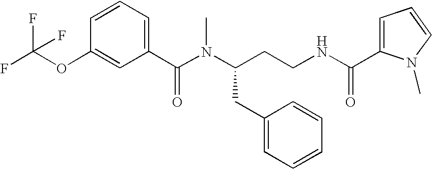

Obtained in analogy to example 4. [LCMS Rt_{F}=1.33 min; [M+H]⁺=474.2]

### Example 124

### 1-Methyl-1H-pyrrole-2-carboxylic acid {(S)-3-[(3-fluoro-5-methyl-benzoyl)-methyl-amino]-4-phenyl-butyl}-amide

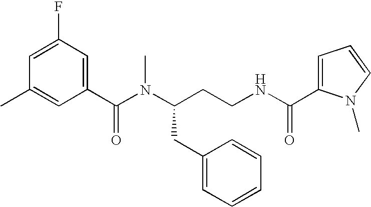

Obtained in analogy to example 4. [LCMS Rt_{F}=1.25 min; [M+H]⁺=422.3]

### Example 125

### 1-Methyl-1H-pyrrole-2-carboxylic acid {(S)-3-[(3,5-dimethyl-benzoyl)-methyl-amino]-4-phenyl-butyl}-amide

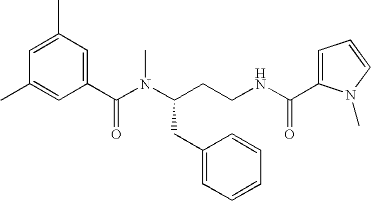

Obtained in analogy to example 4. [LCMS Rt_{F}=1.30 min; [M+H]⁺=418.3]

### Example 126

### 1-Methyl-1H-pyrrole-2-carboxylic acid {(S)-3-methyl-[3-propoxy-benzoyl)-amino]-4-phenyl-butyl}-amide

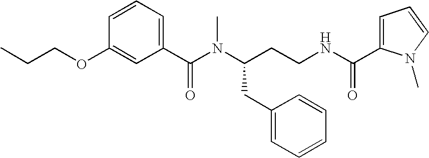

Obtained in analogy to example 4. [LCMS Rt_{F}=1.30 min; [M+H]⁺=418.3]

### Radioligand Binding Assay

For crude cell membrane preparations, cells (CHO, Chinese hamster ovary or HEK, human embryonic kidney) expressing human orexin 1 or human orexin 2 receptors, were washed with HEPES (10 mM, pH 7.5), scraped off the culture plates with the same buffer, and centrifuged at 4° C. for 5 min at 2500×g. The cell pellet was either stored at −80° C. or used directly. Before the experiments, cell membranes were re-suspended in binding assay buffer (10 mM HEPES, 0.5% (w/v) bovine serum albumin, pH 7.5) by homogenisation with a Polytron homogeniser at 50 Hz for 20 s. Cell membranes were also used as made available by commercial providers.

In initial saturation experiments (to calculate Bmax), cell homogenates (150 μl) were incubated with 25-300 pM of the radioligand ([¹²⁵I]orexin A, 50 μl), 8 concentrations in triplicates in the presence or absence Orexin A (1 μM, 50 μl) to define non specific binding. Bound radioactivity was measured, and data were analysed with the program XLFIT or Graphpad Prism. Protein concentration was determined according to the Bradford/BioRad Protein Assay Kit.

In competition experiments, cell homogenates (150 μl) were incubated in assay buffer (10 mM HEPES, pH 7.5, 0.5% (w/v) bovine serum albumin, 5 mM MgCl₂, 1 nnMCaCl₂, and tween 0.05%) for 1 h at room temperature with about 100 μM of the radioligand ([¹²⁵I]orexin A, 2100 Ci/mmole, 50 μl), and with various concentrations of compounds of the invention (50 μl) in triplicates; non-specific binding was determined in the presence of Orexin A (1 μM). Reactions were terminated by vacuum filtration, 3 washes of ice cold wash buffer (Tris-HCl pH 7.4/10 mM, with NaCl 154 mM). Competition data is expressed in Table 1 as Kd [μM].

### Calcium Accumulation in Cells (FLIPR):

Cells expressing human orexin 1 or human orexin 2 receptors, were seeded at 8,000 cells/well in 384 well black-walled clear bottom, poly-D-lysine coated plates. After 24 h, the medium was removed and cells were washed once with phosphate buffered saline and serum-deprived overnight in assay buffer (130 mM NaCl, 5.4 mM KCl, 1.8 mM CaCl₂, 0.8 mM MgSO₄, 0.9 mM NaH₂PO₄, 25 mM glucose, 20 mM HEPES, pH 7.4) containing bovine serum albumin (1% w/v).

On the day of the experiment, the cells seeded in black plates were treated with assay buffer containing the Ca²⁺ sensitive fluorescent dye Fluo-4-AM (2 μM), and probenecid (0.1 mM). After 1 h plates were washed twice with, and resuspended in, assay buffer containing probenecid (0.1 mM) using a multi plate washer. The plates were placed into a FLIPR II (Fluorometric Imaging Plate Reader, Molecular Devices, Sunnyvale, Calif., USA) and baseline fluorescence (fluorescence light units, FLU) was measured (5 measurements, 2 S each; laser excitation 488 nm at 0.6-1 W, CCD camera exposure 0.4 s) before addition of buffer alone (basal) or containing test compounds (either compound of formula I alone, agonist alone or agonist in the presence of various concentrations of compounds of formula I). Fluorescence measurements were then continued every 1 S for 120 S followed by every 4 S for 240 S.

The measurements were typically made in two sequences:

In the first round, compounds of formula I were tested alone, to confirm that they do not display any/any significant agonist activity. Compounds of formula I were tested usually in a concentration range from 10⁻⁹ M to 10⁻⁵ M.

In the second round, performed one hour later (to allow for equilibration), Orexin A was tested either in the absence (calibration curves, Orexin A agonist controls) or in the presence of compounds of formula I to determine antagonism.

Inhibition data is expressed in Table 1 as K_{d} [μM], converted by the Cheng and Prusoff correction (Kd=IC₅₀/1+(LIEC₅₀)), where IC₅₀ is the 50% inhibition value determined in concentration response inhibition curves, EC₅₀ is the half maximal activation concentration determined for orexin A in concentration response curves and L is the concentration of orexin A used in inhibition experiments performed in with a submaximal concentration of orexin A in the presence of up to 8 increasing concentrations of compound of formula I.

Inhibition data is also expressed in Table 1 as % inhibition value measured at a concentration of 10 μM of compound of formula I.

TABLE 1

| | FLIPR | FLIPR | binding | binding |
|---|---|---|---|---|
| | hOx1R | hOx2R_{—} | hOx1R | hOx2R |
| | Kd [μM] | Kd [μM] | Kd [μM] | Kd [μM] |
| Example 1 | 0.228 | 0.034 | 0.179 | 0.063 |
| Example 2 | 4 ^{a} | 19 ^{a} | >10 | 4.97 |
| Example 3 | 0.048 | 0.003 | 0.017 | 0.010 |
| Example 4 | 0.173 | 0.044 | 0.112 | 0.014 |
| Example 5 | 0.511 | 0.008 | 0.999 | 0.018 |
| Example 6 | 0.031 | 0.057 | 0.025 | 0.020 |
| Example 7 | 0.016 | 0.040 | 0.007 | 0.041 |
| Example 8 | 0.211 | 0.032 | 0.270 | 0.046 |
| Example 9 | 1.40 | 0.145 | 0.474 | 0.046 |
| Example 10 | 1.36 | 0.027 | 3.27 | 0.059 |
| Example 11 | 0.022 | 0.016 | 0.021 | 0.052 |
| Example 12 | 0.437 | 0.040 | 1.07 | 0.098 |
| Example 13 | 2.21 | 0.074 | 0.810 | 0.107 |
| Example 14 | 0.609 | 0.106 | 1.07 | 0.521 |
| Example 15 | 17 ^{a} | 0.272 | n.d. | n.d. |
| Example 16 | 1.51 | 0.044 | >10 | 0.074 |
| Example 17 | 36 ^{a} | 0.041 | 8.79 | 0.107 |
| Example 18 | 0.100 | 0.014 | 0.332 | 0.129 |
| Example 19 | 1.12 | 0.114 | 0.793 | 0.131 |
| Example 20 | 0.413 | 0.123 | 0.163 | 0.168 |
| Example 21 | 0.070 | 0.174 | 0.062 | 0.203 |
| Example 22 | 1.57 | 0.105 | >10 | 0.214 |
| Example 23 | 0.152 | 0.419 | 0.052 | 0.265 |
| Example 24 | 0.124 | 0.005 | 1.39 | 0.273 |
| Example 25 | 1.00 | 0.036 | 2.22 | 0.308 |
| Example 26 | 0.122 | 0.010 | 3.28 | 0.308 |
| Example 27 | 0.076 | 0.011 | 0.783 | 0.337 |
| Example 28 | 0.028 | 0.004 | 0.579 | 0.371 |
| Example 29 | 2.19 | 0.157 | 0.518 | 0.374 |
| Example 30 | 2.19 | 0.897 | 1.10 | 0.396 |
| Example 31 | 0.057 | 0.043 | 0.164 | 0.410 |
| Example 32 | 24 ^{a} | 0.067 | >10 | 0.435 |
| Example 33 | 1.96 | 0.076 | 5.73 | 0.450 |
| Example 34 | 0.731 | 0.558 | 0.253 | 0.466 |
| Example 35 | 0.080 | 0.009 | 1.33 | 0.468 |
| Example 36 | 37 ^{a} | 0.565 | 0.853 | 0.468 |
| Example 37 | 0.836 | 0.440 | 0.358 | 0.479 |
| Example 38 | 0.035 | 0.162 | 0.086 | 0.590 |
| Example 39 | 0 ^{a} | 0.419 | >10 | 0.610 |
| Example 40 | 0.193 | 0.221 | 0.125 | 0.619 |
| Example 41 | 1.60 | 0.196 | 1.73 | 0.484 |
| Example 42 | 0.909 | 0.826 | 1.73 | 0.688 |
| Example 43 | 0.941 | 0.203 | 2.25 | 0.607 |
| Example 44 | 0.033 | 0.090 | 0.082 | 0.781 |
| Example 45 | 2.71 | 0.424 | 5.33 | 0.748 |
| Example 46 | 1.83 | 1.00 | 0.397 | 0.870 |
| Example 47 | 6.29 | 1.18 | 12.32 | 0.906 |
| Example 48 | 0.868 | 0.084 | 2.82 | 0.940 |
| Example 49 | 9 ^{a} | 0.414 | 2.45 | 1.07 |
| Example 50 | 0.032 | 0.059 | 0.783 | 1.11 |
| Example 51 | 16 ^{a} | 0.794 | 3.54 | 1.15 |
| Example 52 | 0.466 | 0.916 | 2.42 | 0.963 |
| Example 53 | 13 ^{a} | 0.748 | 3.41 | 1.46 |
| Example 54 | 9 ^{a} | 0.958 | >10 | 1.73 |
| Example 55 | 1.64 | 1.29 | 0.075 | 1.76 |
| Example 56 | 1 ^{a} | 35 ^{a} | >10 | 2.72 |
| Example 57 | 0.012 | 0.026 | 0.443 | 2.85 |
| Example 58 | 1 ^{a} | 1.06 | 2.30 | 3.43 |
| Example 59 | 2.04 | 0.562 | 3.63 | 3.62 |
| Example 60 | 3.47 | 0.362 | 17.51 | 3.86 |
| Example 61 | 3.83 | 0.994 | 4.56 | 3.92 |
| Example 62 | 0.872 | 0.022 | 0.222 | 0.047 |
| Example 63 | 0 ^{a} | 24 ^{a} | >10 | 5.902 |
| Example 64 | 1.01 | 1.07 | 0.540 | 6.859 |
| Example 65 | 3 ^{a} | 41 ^{a} | >10 | 6.980 |
| Example 66 | 0^{a} | 33 ^{a} | >10 | 7.122 |
| Example 67 | 33 ^{a} | 0.180 | 1.085 | 0.114 |
| Example 68 | 3.58 | 2.37 | >10 | 7.53 |
| Example 69 | 3.52 | 1.66 | n.d. | n.d. |
| Example 70 | 0 ^{a} | 29 ^{a} | >10 | 8.313 |
| Example 71 | 1.94 | 28 ^{a} | 1.61 | 9.52 |
| Example 72 | 27 ^{a} | 0.761 | n.d. | n.d. |
| Example 73 | 1.92 | 1.31 | >10 | >10 |
| Example 74 | 3.26 | 1.61 | >10 | >10 |
| Example 75 | 0.267 | 0.031 | 0.705 | 0.473 |
| Example 76 | 0.376 | 0.001 | 0.094 | 0.004 |
| Example 77 | 25 ^{a} | 0.581 | 8.42 | 3.71 |
| Example 78 | 1.47 | 0.028 | 0.660 | 0.024 |
| Example 79 | 0.225 | 0.012 | 0.073 | 0.030 |
| Example 80 | 0.541 | 0.097 | 0.059 | 0.098 |
| Example 81 | 0.012 | 0.013 | 0.006 | 0.023 |
| Example 82 | 0.963 | 0.002 | 0.163 | 0.012 |
| Example 83 | 0.042 | 0.003 | 0.054 | 0.021 |
| Example 84 | 0.050 | 0.809 | 0.030 | 1.33 |
| Example 85 | 0.237 | 0.394 | n.d. | n.d. |
| Example 86 | 4 ^{a} | 1.50 | n.d. | n.d. |
| Example 87 | 4.42 | 1.29 | n.d. | n.d. |
| Example 88 | 19 ^{a} | 3.09 | n.d. | n.d. |
| Example 89 | 5.45 | 2.55 | n.d. | n.d. |
| Example 90 | 1.25 | 2.54 | n.d. | n.d. |
| Example 91 | 16 ^{a} | 0.375 | n.d. | n.d. |
| Example 92 | 2 ^{a} | 0.921 | n.d. | n.d. |
| Example 93 | 30 ^{a} | 0.164 | 3.11 | 0.114 |
| Example 94 | 12 ^{a} | 0.565 | n.d. | n.d. |
| Example 95 | 3.43 | 0.140 | 3.44 | 0.059 |
| Example 96 | 0.474 | 0.074 | 0.247 | 0.133 |
| Example 97 | 0.185 | 0.039 | 0.046 | 0.030 |
| Example 98 | 28 ^{a} | 24 ^{a} | n.d. | n.d. |
| Example 99 | 1.11 | 0.115 | 1.94 | 0.822 |
| Example 100 | 1.08 | 0.131 | n.d. | n.d. |
| Example 101 | 13 ^{a} | 1.68 | n.d. | n.d. |
| Example 102 | 0.018 | 0.001 | 0.028 | 0.002 |
| Example 103 | 1.40 | 0.008 | 0.624 | 0.019 |
| Example 104 | 0.609 | 0.004 | 0.123 | 0.006 |
| Example 105 | 0.811 | 0.007 | 0.286 | 0.012 |
| Example 106 | 0.084 | 0.000 | 0.050 | 0.003 |
| Example 107 | 1.07 | 0.054 | 2.51 | 0.162 |
| Example 108 | 3.99 | 0.542 | n.d. | n.d. |
| Example 109 | 39 ^{a} | 1.72 | n.d. | n.d. |
| Example 110 | 1.73 | 0.090 | 1.38 | 0.399 |
| Example 111 | 25 ^{a} | 0.562 | n.d. | n.d. |
| Example 112 | 0.415 | 0.498 | 0.477 | 1.16 |
| Example 113 | 24 ^{a} | 0.497 | n.d. | 0.531 |
| Example 114 | 34 ^{a} | 18 ^{a} | n.d. | n.d. |
| Example 115 | 13 ^{a} | 33 ^{a} | n.d. | n.d. |
| Example 116 | 1.55 | 29 ^{a} | n.d. | n.d. |
| Example 117 | 0.987 | 0.065 | 0.444 | 0.081 |
| Example 118 | 1.07 | 0.055 | 0.977 | 0.076 |
| Example 119 | 0.534 | 0.004 | 0.249 | 0.014 |
| Example 120 | 0.182 | 0.004 | n.d. | n.d. |
| Example 121 | 0.696 | 0.002 | 0.206 | 0.008 |
| Example 122 | 0.773 | 0.024 | 0.333 | 0.021 |
| Example 123 | 0.865 | 0.007 | 1.58 | 0.030 |
| Example 124 | 0.710 | 0.013 | 0.599 | 0.039 |
| Example 125 | 0.230 | 0.002 | 0.176 | 0.007 |
| Example 126 | 2.10 | 0.056 | 2.48 | 0.087 |

n.d. = not determined
^{a} % inhibition value measured at a concentration of 10 μM of compound of formula I.

In one embodiment, the invention provides a method of inhibiting orexin receptor activity in a subject, wherein the method comprises administering to the subject a therapeutically effective amount of a compound of formula I.

In a further embodiment, the invention provides a method of treating a disorder or a disease in a subject mediated by orexin receptors, wherein the method comprises administering to the subject a therapeutically effective amount of a compound of formula I. Preferably said disorder or said disease is selected from sleep disorders, eating disorders, substance-related disorders or Alzheimers disease.

In yet a further embodiment, the invention provides the use of a compound of formula I, for the treatment of a disorder or disease in a subject mediated by orexin receptors.

In yet a further embodiment, the invention provides the use of a compound of formula I, for the treatment of a disorder or disease in a subject characterized by an abnormal activity of orexin receptors. Preferably said disorder or said disease is selected from sleep disorders, eating disorders, substance-related disorders or Alzheimers disease.

1. A compound of the formula I 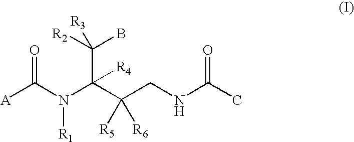
wherein
R₁ is C₁₋₆alkyl, C₁₋₆halogenalkyl, C₃₋₆cycloalkyl or C₃₋₆cycloalkyl(C₁₋₄alkyl);
R₂, R₃, R₅ and R₆ are each independently selected from hydrogen, halogen, hydroxyl, C₁₋₆alkyl, C₁₋₆halogenalkyl, C₃₋₆cycloalkyl, C₃₋₆cycloalkyl(C₁₋₄alkyl), C₁₋₆alkoxy, or C₁₋₆halogenalkoxy;
or R₂ and R₃ together are oxo;
or R₂ and R₃ taken together with the carbon atom to which they are bound form a C₃₋₆cycloalkyl;
or R₅ and R₆ together are oxo;
or R₅ and R₆ taken together with the carbon atom to which they are bound form a C₃₋₆cycloalkyl;
R₄ is hydrogen, C₁₋₆alkyl or hydroxyl;
A is phenyl, which may be substituted once or twice by R₇;
each R₇ independently is halogen, C₁₋₆alkyl, C₁₋₆halogenalkyl, C₃₋₆cycloalkyl, C₃₋₆cycloalkyl(C₁₋₄alkyl), C₁₋₆alkoxy, or C₁₋₆halogenalkoxy;
or two R₇ at adjacent ring atoms form a C₃₋₄alkylene group, wherein 1-2 carbon atoms may be replaced by X₁, and wherein the C₃₋₄alkylene group may be substituted once or more than once by R₈;
each X₁ independently is —O— or —N(R₉)—;
each R₉ independently is hydrogen or C₁₋₆alkyl;
each R₈ independently is halogen or C₁₋₆alkyl;
or two R₇ at adjacent ring atoms are —CH═CH—CH═CH—;
or two R₇ at adjacent ring atoms are —CH═CH—X₂—;
X₂ is —O— or —N(R₁₀)—;
R₁₀ is hydrogen or C₁₋₆alkyl;
B is 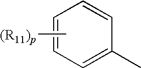
p is 0 or 1;
R₁₁ is halogen, C₁₋₆alkyl, C₃₋₆cycloalkyl, C₁₋₆alkoxy, or C₁₋₆halogenalkoxy; and
C is a five- to ten-membered monocyclic or fused polycyclic aromatic ring system which may contain from 1 to 4 hetero atoms selected from nitrogen, oxygen and sulfur, wherein the ring system may contain not more than 2 oxygen atoms and not more than 2 sulfur atoms, and wherein the ring system may be substituted once or more than once by R₁₂, and wherein a substituent on a nitrogen in a heterocyclic ring system may not be halogen;
each R₁₂ independently is C₁₋₆alkl, C₁₋₆halogenalkyl, C₁₋₆alkoxy, C₁₋₆halogenalkoxy, halogen, cyano or a three- to six-membered monocyclic ring system which may be aromatic, saturated or partially saturated and which may contain from 1 to 4 hetero atoms selected from nitrogen, oxygen and sulfur, and wherein each ring system may contain not more than 2 oxygen atoms and not more than 2 sulfur atoms, and wherein each ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen;
or two R₁₂ at adjacent ring atoms form a C₃₋₄alkylene group, wherein 1-2 carbon atoms may be replaced by X₃, and wherein the C₃₋₄alkylene group may be substituted once or more than once by R₁₃;
each X₃ independently is —O— or —N(R₁₄)—;
each R₁₄ independently is hydrogen or C₁₋₆alkyl;
each R₁₃ independently is halogen or C₁₋₆alkyl;
or C is C₁₋₆alkyl, C₁₋₆halogenalkyl, C₃₋₆cycloalkyl or C₃₋₆cycloalkyl(C₁₋₄alkyl);
in free form or in salt form.

2. A compound of formula I according to claim 1, wherein said compound is a compound of formula IA 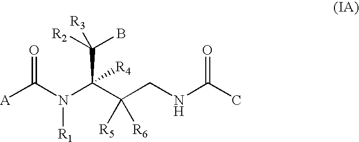
wherein R₁, R₂, R₃, R₄, R₅, R₆, A, B and C are as defined according to claim 1.

3. A compound of formula I according to claim 1, wherein R₁ is C₁₋₆alkyl and R₂, R₃, R₄, R₅ and R₆ are each hydrogen.

4. A compound of formula I according to claim 1, wherein A is a ring system selected from 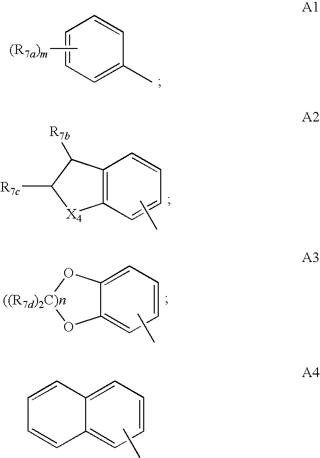
m is 0, 1 or 2;
each R₇ₐ independently is halogen, C₁₋₆alkyl, C₁₋₆halogenalkyl, C₃₋₆cycloalkyl, C₃₋₆cycloalkyl(C₁₋₄alkyl), C₁₋₆alkoxy, or C₁₋₆halogenalkoxy;
R_{7b} and R_{7c} are each independently hydrogen or C₁₋₆alkyl, or are together a bond;
X₄ is oxygen or —N(R₇ₑ)—;
R₇ₑ is hydrogen or C₁₋₆alkyl;
n is 1 or 2;
each R_{7d} is independently hydrogen, halogen or C₁₋₆alkyl.

5. A compound of formula I according to claim 1, wherein p is 0.

6. A compound of formula I according to claim 1, wherein C is a five- to ten-membered monocyclic or fused polycyclic aromatic ring system which may contain from 1 to 4 hetero atoms selected from nitrogen, oxygen and sulfur, wherein the ring system may contain not more than 2 oxygen atoms and not more than 2 sulfur atoms, and wherein the ring system may be substituted once or more than once by R₁₂, and wherein a substituent on a nitrogen in a heterocyclic ring system may not be halogen;
each R₁₂ independently is C₁₋₆alkyl, C₁₋₆halogenalkyl, C₁₋₆alkoxy, C₁₋₆halogenalkoxy, halogen, cyano or a three- to six-membered monocyclic ring system which may be aromatic, saturated or partially saturated and which may contain from 1 to 4 hetero atoms selected from nitrogen, oxygen and sulfur, and wherein each ring system may contain not more than 2 oxygen atoms and not more than 2 sulfur atoms, and wherein each ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen;
or two R₁₂ at adjacent ring atoms form a C₃₋₄alkylene group, wherein 1-2 carbon atoms may be replaced by X₃, and wherein the C₃₋₄alkylene group may be substituted once or more than once by R₁₃;
each X₃ independently is —O— or —N(R₁₄)—;
each R₁₄ independently is hydrogen or C₁₋₆alkyl;
each R₁₃ independently is halogen or C₁₋₆alkyl.

7. A compound of formula I according to claim 1, wherein said compound is selected from the group consisting of
Pyridine-2-carboxylic acid {3-[(benzo[1,3]dioxole-5-carbonyl)-methyl-amino]-4-phenyl-butyl}-amide;
Quinoline-4-carboxylic acid [3-(benzoyl-methyl-amino)-4-phenyl-butyl]-amide;
1-Methyl-1H-benzoimidazole-2-carboxylic acid {3-[(1H-indole-4-carbonyl)-methyl-amino]-4-phenyl-butyl}-amide;
Pyridine-2-carboxylic acid {3-[methyl-(3-trifluoromethyl-benzoyl)-amino]-4-phenyl-butyl}-amide;
Pyridine-2-carboxylic acid {3-[(3,5-bis-trifluoromethyl-benzoyl)-methyl-amino]-4-phenyl-butyl}-amide;
Pyridine-2-carboxylic acid {3-[methyl-(naphthalene-1-carbonyl)-amino]-4-phenyl-butyl}-amide;
1H-Indole-5-carboxylic acid {1-benzyl-3-[(pyridine-2-carbonyl)-amino]-propyl}-methyl-amide;
Pyridine-2-carboxylic acid {3-[(benzo[1,3]dioxole-5-carbonyl)-ethyl-amino]-4-phenyl-butyl}-amide;
Pyridine-2-carboxylic acid {3-[(3,4-dimethoxy-benzoyl)-methyl-amino]-4-phenyl-butyl}-amide;
Pyridine-2-carboxylic acid {3-[(3,5-bis-trifluoromethyl-benzoyl)-ethyl-amino]-4-phenyl-butyl}-amide;
1H-Indole-4-carboxylic acid {1-benzyl-3-[(pyridine-2-carbonyl)-amino]-propyl}-methyl-amide;
1-Methyl-1H-benzoimidazole-2-carboxylic acid {3-[(3,5-bis-trifluoromethyl-benzoyl)-methyl-amino]-4-phenyl-butyl}-amide;
Pyridine-2-carboxylic acid {3-[(benzo[1,3]dioxole-5-carbonyl)-propyl-amino]-4-phenyl-butyl}-amide;
1H-Indole-2-carboxylic acid {3-[(benzo[1,3]dioxole-5-carbonyl)-methyl-amino]-4-phenyl-butyl}-amide;
N-[3-(Benzoyl-methyl-amino)-4-phenyl-butyl]-benzamide;
Pyridine-2-carboxylic acid [3-[(3,5-bis-trifluoromethyl-benzoyl)-methyl-amino]-4-(4-chloro-phenyl)-butyl]-amide;
N-(3-Benzoylamino-1-benzyl-propyl)-N-methyl-3,5-bis-trifluoromethyl-benzamide;
1-Methyl-1H-benzoimidazole-2-carboxylic acid {3-[methyl-(naphthalene-1-carbonyl)-amino]-4-phenyl-butyl}-amide;
1-Methyl-1H-benzoimidazole-2-carboxylic acid {3-[(benzo[1,3]dioxole-4-carbonyl)-methyl-amino]-4-phenyl-butyl}-amide;
Pyridine-2-carboxylic acid {3-[(benzofuran-5-carbonyl)-methyl-amino]-4-phenyl-butyl}-amide;
Pyridine-2-carboxylic acid {3-[methyl-(naphthalene-2-carbonyl)-amino]-4-phenyl-butyl}-amide;
Pyridine-2-carboxylic acid {3-[(3,5-bis-trifluoromethyl-benzoyl)-propyl-amino]-4-phenyl-butyl}-amide;
2,3-Dihydro-1H-indole-5-carboxylic acid {1-benzyl-3-[(pyridine-2-carbonyl)-amino]-propyl}-methyl-amide;
1-Methyl-1H-benzoimidazole-2-carboxylic acid {4-(4-fluoro-phenyl)-3-[methyl-(naphthalene-1-carbonyl)-amino]-butyl}-amide;
Pyridine-2-carboxylic acid [3-[(benzo[1,3]dioxole-5-carbonyl)-methyl-amino]-4-(4-chloro-phenyl)-butyl]-amide;
1-Methyl-1H-benzoimidazole-2-carboxylic acid {4-(4-chloro-phenyl)-3-[methyl-(naphthalene-1-carbonyl)-amino]-butyl}-amide;
5-(2-Fluoro-phenyl)-2-methyl-thiazole-4-carboxylic acid [3-(benzoyl-methyl-amino)-4-phenyl-butyl]-amide;
Pyridine-2-carboxylic acid {4-(4-fluoro-phenyl)-3-[methyl-(naphthalene-1-carbonyl)-amino]-butyl}-amide;
Pyridine-2-carboxylic acid {3-[(2,3-dihydro-benzofuran-5-carbonyl)-methyl-amino]-4-phenyl-butyl}-amide;
Pyridine-2-carboxylic acid {3-[(benzo[1,3]dioxole-4-carbonyl)-methyl-amino]-4-phenyl-butyl}-amide;
1-Methyl-1H-benzoimidazole-2-carboxylic acid {3-[(benzo[1,3]dioxole-5-carbonyl)-methyl-amino]-4-phenyl-butyl}-amide;
Benzo[1,3]dioxole-4-carboxylic acid [3-(benzoyl-methyl-amino)-4-phenyl-butyl]-amide;
6-Methyl-imidazo[2,1-b]thiazole-5-carboxylic acid [3-(benzoyl-methyl-amino)-4-phenyl-butyl]-amide;
Pyridine-2-carboxylic acid {3-[(3,5-difluoro-benzoyl)-methyl-amino]-4-phenyl-butyl}-amide;
Pyridine-2-carboxylic acid {4-(4-chloro-phenyl)-3-[methyl-(naphthalene-1-carbonyl)-amino]-butyl}-amide;
1-Methyl-1H-indole-7-carboxylic acid {1-benzyl-3-[(pyridine-2-carbonyl)-amino]-propyl}-methyl-amide;
Pyridine-2-carboxylic acid [3-(benzoyl-methyl-amino)-4-phenyl-butyl]-amide;
1H-Indole-7-carboxylic acid {1-benzyl-3-[(pyridine-2-carbonyl)-amino]-propyl}-methyl-amide;
N-(1-Benzyl-3-pentanoylamino-propyl)-N-methyl-3,5-bis-trifluoromethyl-benzamide;
1-Methyl-1H-indole-4-carboxylic acid {1-benzyl-3-[(pyridine-2-carbonyl)-amino]-propyl}-methyl-amide;
Pyridine-2-carboxylic acid {3-[(3,5-dimethoxy-benzoyl)-methyl-amino]-4-phenyl-butyl}-amide;
Pyridine-2-carboxylic acid {3-[(4-methoxy-benzoyl)-methyl-amino]-4-phenyl-butyl}-amide;
1-Methyl-1H-benzoimidazole-2-carboxylic acid {3-[methyl-(naphthalene-2-carbonyl)-amino]-4-phenyl-butyl}-amide;
Pyridine-2-carboxylic acid {3-[(benzo[1,3]dioxole-4-carbonyl)-ethyl-amino]-4-phenyl-butyl}-amide;
Pyridine-2-carboxylic acid {3-[(2,3-dihydro-benzofuran-7-carbonyl)-methyl-amino]-4-phenyl-butyl}-amide;
1-Methyl-1H-benzoimidazole-2-carboxylic acid [3-(benzoyl-methyl-amino)-4-phenyl-butyl]-amide;
Quinoline-8-carboxylic acid [3-(benzoyl-methyl-amino)-4-phenyl-butyl]-amide;
1-Methyl-1H-benzoimidazole-2-carboxylic acid {4-(4-chloro-phenyl)-3-[methyl-(naphthalene-2-carbonyl)-amino]-butyl}-amide;
Pyridine-2-carboxylic acid [3-[(benzo[1,3]dioxole-5-carbonyl)-methyl-amino]-4-(4-fluoro-phenyl)-butyl]-amide;
Pyridine-2-carboxylic acid {3-[(3-methoxy-benzoyl)-methyl-amino]-4-phenyl-butyl}-amide;
Pyridine-2-carboxylic acid {4-(4-chloro-phenyl)-3-[methyl-(naphthalene-2-carbonyl)-amino]-butyl}-amide;
Pyridine-2-carboxylic acid {3-[(2,3-dihydro-benzo[1,4]dioxine-5-carbonyl)-methyl-amino]-4-phenyl-butyl}-amide;
1-Methyl-2,3-dihydro-1H-indole-5-carboxylic acid {1-benzyl-3-[(pyridine-2-carbonyl)-amino]-propyl}-methyl-amide;
1-Methyl-1H-benzoimidazole-2-carboxylic acid [3-[(benzo[1,3]dioxole-4-carbonyl)-methyl-amino]-4-(4-fluoro-phenyl)-butyl]-amide;
1-Methyl-1H-benzoimidazole-2-carboxylic acid {4-(4-fluoro-phenyl)-3-[methyl-(naphthalene-2-carbonyl)-amino]-butyl}-amide;
Pyridine-2-carboxylic acid {4-(4-fluoro-phenyl)-3-[methyl-(naphthalene-2-carbonyl)-amino]-butyl}-amide;
Pyridine-2-carboxylic acid {3-[(benzo[1,3]dioxole-4-carbonyl)-propyl-amino]-4-phenyl-butyl}-amide;
1-Methyl-1H-indole-5-carboxylic acid {3-[(3,5-bis-trifluoromethyl-benzoyl)-methyl-amino]-4-phenyl-butyl}-amide;
Benzo[1,3]dioxole-5-carboxylic acid {3-[(benzo[1,3]dioxole-5-carbonyl)-amino]-1-benzyl-propyl}-methyl-amide;
Pyridine-2-carboxylic acid [3-[(benzo[1,3]dioxole-4-carbonyl)-methyl-amino]-4-(4-fluoro-phenyl)-butyl]-amide;
1H-Indole-6-carboxylic acid {1-benzyl-3-[(pyridine-2-carbonyl)-amino]-propyl}-methyl-amide;
Pyridine-2-carboxylic acid [3-[(benzo[1,3]dioxole-4-carbonyl)-methyl-amino]-4-(4-chloro-phenyl)-butyl]-amide;
Pyridine-2-carboxylic acid [3-(benzoyl-methyl-amino)-4-(4-fluoro-phenyl)-butyl]-amide;
2,3-Dihydro-benzofuran-7-carboxylic acid [3-(benzoyl-methyl-amino)-4-phenyl-butyl]-amide;
Pyridine-2-carboxylic acid [3-(benzoyl-methyl-amino)-2-hydroxy-4-phenyl-butyl]-amide;
Pyridine-2-carboxylic acid [3-(benzoyl-methyl-amino)-4-(4-chloro-phenyl)-butyl]-amide;
Pyridine-2-carboxylic acid {3-[(2,3-dihydro-benzo[1,4]dioxine-6-carbonyl)-methyl-amino]-4-phenyl-butyl}-amide;
Benzo[1,3]dioxole-5-carboxylic acid {3-[(benzo[1,3]dioxole-5-carbonyl)-methyl-amino]-2-hydroxy-4-phenyl-butyl}-amide;
Pyridine-2-carboxylic acid {3-[methyl-(4-trifluoromethyl-benzoyl)-amino]-4-phenyl-butyl}-amide;
1-Methyl-1H-indole-2-carboxylic acid {3-[(benzo[1,3]dioxole-5-carbonyl)-methyl-amino]-4-phenyl-butyl}-amide;
1-Methyl-1H-indole-2-carboxylic acid {3-[(benzo[1,3]dioxole-5-carbonyl)-methyl-amino]-4-phenyl-butyl}-amide;
Pyridine-2-carboxylic acid {3-[(3,4-bis-trifluoromethyl-benzoyl)-methyl-amino]-4-phenyl-butyl}-amide;
1-Methyl-1H-imidazole-2-carboxylic acid {3-[(benzo[1,3]dioxole-5-carbonyl)-methyl-amino]-4-phenyl-butyl}-amide;
1-Methyl-1H-benzoimidazole-2-carboxylic acid {3-[(3-methoxy-benzoyl)-methyl-amino]-4-phenyl-butyl}-amide;
1-Methyl-1H-benzoimidazole-2-carboxylic acid {3-[(3,4-dimethoxy-benzoyl)-methyl-amino]-4-phenyl-butyl}-amide;
1-Methyl-1H-benzoimidazole-2-carboxylic acid {3-[(1H-indole-5-carbonyl)-methyl-amino]-4-phenyl-butyl}-amide;
6-Methyl-imidazo[2,1-b]thiazole-5-carboxylic acid {3-[(benzo[1,3]dioxole-5-carbonyl)-methyl-amino]-4-phenyl-butyl}-amide;
5-(2-Fluoro-phenyl)-2-methyl-thiazole-4-carboxylic acid {3-[(benzo[1,3]dioxole-5-carbonyl)-methyl-amino]-4-phenyl-butyl}-amide;
1-Methyl-1H-indole-5-carboxylic acid {1-benzyl-3-[(pyridine-2-carbonyl)-amino]-propyl}-methyl-amide;
1-Methyl-1H-indole-6-carboxylic acid {1-benzyl-3-[(pyridine-2-carbonyl)-amino]-propyl}-methyl-amide; N-[3-(Benzoyl-methyl-amino)-4-phenyl-butyl]-nicotinamide;
Quinoline-2-carboxylic acid [3-(benzoyl-methyl-amino)-4-phenyl-butyl]-amide;
Quinoline-7-carboxylic acid [3-(benzoyl-methyl-amino)-4-phenyl-butyl]-amide;
Isoquinoline-3-carboxylic acid [3-(benzoyl-methyl-amino)-4-phenyl-butyl]-amide;
1-Methyl-1H-benzoimidazole-2-carboxylic acid {3-[(4-methoxy-benzoyl)-methyl-amino]-4-phenyl-butyl}-amide;
1-Methyl-1H-pyrrole-2-carboxylic acid [3-(benzoyl-methyl-amino)-4-phenyl-butyl]-amide;
1-Methyl-1H-imidazole-2-carboxylic acid [3-(benzoyl-methyl-amino)-4-phenyl-butyl]-amide;
Benzo[1,3]dioxole-5-carboxylic acid [3-(benzoyl-methyl-amino)-4-phenyl-butyl]-amide;
2,3-Dihydro-benzofuran-5-carboxylic acid [3-(benzoyl-methyl-amino)-4-phenyl-butyl]-amide;
Pyridine-2-carboxylic acid [3-(benzoyl-methyl-amino)-2-hydroxy-4-phenyl-butyl]-amide;
Pyridine-2-carboxylic acid {3-[methyl-(3-methyl-benzoyl)-amino]-4-phenyl-butyl}-amide;
Pyridine-2-carboxylic acid {3-[(3,5-dimethyl-benzoyl)-methyl-amino]-4-phenyl-butyl}-amide;
Pyridine-2-carboxylic acid {3-[(2,6-dimethyl-benzoyl)-methyl-amino]-4-phenyl-butyl}-amide;
Pyridine-2-carboxylic acid {3-[(4-bromo-benzoyl)-methyl-amino]-4-phenyl-butyl}-amide;
Pyridine-2-carboxylic acid {3-[(2-bromo-benzoyl)-methyl-amino]-4-phenyl-butyl}-amide;
Pyridine-2-carboxylic acid {3-[(2,2-dimethyl-benzo[1,3]dioxole-5-carbonyl)-methyl-amino]-4-phenyl-butyl}-amide;
1-Methyl-1H-benzoimidazole-2-carboxylic acid {3-[(3-bromo-benzoyl)-methyl-amino]-4-phenyl-butyl}-amide;
1-Methyl-1H-pyrrole-2-carboxylic acid {3-[(3-methoxy-benzoyl)-methyl-amino]-4-phenyl-butyl}-amide;
1-Methyl-1H-pyrrole-2-carboxylic acid {3-[(3,5-dimethoxy-benzoyl)-methyl-amino]-4-phenyl-butyl}-amide;
1-Methyl-1H-pyrrole-2-carboxylic acid {3-[(3-fluoro-5-methoxy-benzoyl)-methyl-amino]-4-phenyl-butyl}-amide;
1-Methyl-1H-pyrrole-2-carboxylic acid {3-[(3-chloro-5-methoxy-benzoyl)-methyl-amino]-4-phenyl-butyl}-amide;
1-Methyl-1H-pyrrole-2-carboxylic acid {3-[(2,2-difluoro-benzo[1,3]dioxole-5-carbonyl)-methyl-amino]-4-phenyl-butyl}-amide;
Benzooxazole-2-carboxylic acid {3-[(benzo[1,3]dioxole-5-carbonyl)-methyl-amino]-4-phenyl-butyl}-amide;
Benzo[1,3]dioxole-5-carboxylic acid (3-acetylamino-1-benzyl-propyl)-methyl-amide;
Pyridine-2-carboxylic acid {3-[(benzo[1,3]dioxole-5-carbonyl)-methyl-amino]-4,4-dideutero-4-phenyl-butyl}-amide;
Pyridine-2-carboxylic acid [3-(benzoyl-methyl-amino)-3-methyl-4-phenyl-butyl]-amide;
Pyridine-2-carboxylic acid [3-(benzoyl-methyl-amino)-2-methyl-4-phenyl-butyl]-amide;
Pyridine-2-carboxylic acid {3-[(benzo[1,3]dioxole-5-carbonyl)-methyl-amino]-2,2-dimethyl-4-phenyl-butyl}-amide;
Pyridine-2-carboxylic acid [3-(benzoyl-methyl-amino)-4-methyl-4-phenyl-pentyl]-amide;
Pyridine-2-carboxylic acid [3-(benzoyl-methyl-amino)-4-hydroxy-4-phenyl-butyl]-amide; and
Pyridine-2-carboxylic acid [3-(benzoyl-methyl-amino)-4-fluoro-4-phenyl-butyl]-amide.

8. A pharmaceutical composition comprising a therapeutically effective amount of a compound according to claim 1 and one or more pharmaceutically acceptable carriers.

9. A combination comprising a therapeutically effective amount of the compound according to claim 1 and one or more therapeutically active agents.

10. A method of inhibiting orexin receptor activity in a subject, wherein the method comprises administering to the subject a therapeutically effective amount of the compound according to claim 1.

11. A method of treating a disorder or a disease in a subject mediated by orexin receptors, wherein the method comprises administering to the subject a therapeutically effective amount of the compound according to claim 1.

12. A compound according to claim 1, for use as a medicament.

13. Use of a compound according to claim 1, for the treatment of a disorder or disease in a subject mediated by orexin receptors.

14. Use of a compound according to claim 1, for the treatment of a disorder or disease in a subject characterized by an abnormal activity of orexin receptors.

15. A compound of formula IX 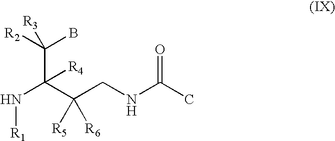
wherein R₁, R₂, R₃, R₄, R₅, R₆, B and C are as defined according to claim 1.

16. A compound of formula XIII 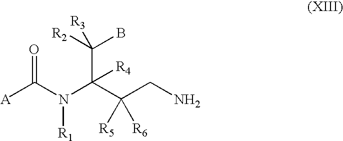
wherein R₁, R₂, R₃, R₄, R₅, R₆, A and B are as defined according to claim 1.
